(19) United States
(12) Patent Application Publication
(10) Pub. No.: US 2012/0150029 A1
(43) Pub. Date: Jun. 14, 2012

(54) System and Method for Detection and Monitoring of Ocular Diseases and Disorders using Optical Coherence Tomography

(75) Inventor: Delia Debuc, Pembroke Pines, FL (US)

(73) Assignee: UNIVERSITY OF MIAMI, Miami, FL (US)

(21) Appl. No.: 13/370,983

(22) Filed: Feb. 10, 2012

Related U.S. Application Data
(63) Continuation-in-part of application No. 13/130,387, filed on Jul. 25, 2011.
(60) Provisional application No. 61/139,082, filed on Dec. 19, 2008.

(51) Int. Cl. A61B 6/00

(52) U.S. Cl. 600/425

(57) ABSTRACT

A system for the imaging, processing and evaluation of tissues provides prognostic and diagnostic details regarding diseased tissue. A set of quantitative measures were developed and integrated in an image-base analysis software tool designed for OCT images. The system and methods in this invention is significant because it allows assessing the optical properties and structure morphology differences between normal healthy subjects and patients with ocular diseases and disorders.

### CROSS-REFERENCE TO RELATED APPLICATIONS

This application is a continuation-in-part of U.S. patent application Ser. No. 13/130,387, filed May 20, 2011, which is a §371 national phase entry of International Application No. PCT/US2009/068653, filed Dec. 18, 2009, which claims priority to U.S. Provisional Patent Application No. 61/139,082, filed Dec. 19, 2008, the entire contents of which are incorporated herein by reference.

### FIELD OF THE INVENTION

Embodiments of the invention relate to the field of ophthalmology. More particularly, the present invention relates to a system for detecting images and methods for characterizing ocular diseases and disorders from optical coherence tomography images.

### BACKGROUND

Diabetes mellitus is a leading cause of vision loss in industrialized and developed countries. Diabetic retinopathy (DR) is a common complication of type 1 diabetes, affecting up to 60% of individuals with type 1 diabetes with a duration of ≧15 years at any point in time. The Diabetes Control and Complications Trial (DCCT) demonstrated that 10% of patients with good metabolic control (glycated hemoglobin ≦6.87%) developed retinopathy, whereas 43% of patients with poor metabolic control (glycated hemoglobin ≧9.49%) did not develop retinopathy. These data suggest that although poor glycemic control is an important predictor of retinopathy, there are many individuals who develop retinopathy despite good glycemic control. Identifying additional predictors of retinopathy is therefore important in screening for the development of this complication.

In diseases in which diagnosis is based mainly on an image, as in DR, not only the contribution of new imaging technologies is essential, but also the development of quantitative computed-assisted tools to aid in the diagnoses is fundamental for the establishment of methods in clinical practice to assist physicians and for improving the quality of medical care. Even though, detailed and well-documented diagnostic protocols have been developed over the last 20 years, there are still constrains in the underlying data generation mechanisms found in actual diabetic screening. Image quality has also been identified as a limited factor.

### SUMMARY

This Summary is provided to briefly indicate the nature and substance of the invention. It is submitted with the understanding that it will not be used to interpret or limit the scope or meaning of the claims.

The aspects and embodiments of the invention disclosed herein relate to a computer-aided diagnostic system and methods for the early detection and diagnosis of neurodegenerative diseases or disorders, including ocular and non-ocular diseases.

In preferred embodiments, the optical properties and structure morphology differences between normal healthy subjects and patients with neurodegenerative diseases are assessed.

In another preferred embodiment, a method of quantifying the various cellular layers of the retina in a subject comprises a computer implemented system. The system comprises an input for receiving source data from an OCT device, and a processing unit configured to perform a methodology according to embodiments of the present invention.

In another preferred embodiment, a method of screening a population for diabetic retinopathy comprises analyzing abnormalities in the structure of the intraretinal layers of individuals in the population using the system and methods according to embodiments of the present invention. In one aspect, the present invention also provides a computer readable medium encoding instructions for performing an image analysis method according to embodiments of the present invention.

Other aspects are described infra.

### BRIEF DESCRIPTION OF THE DRAWINGS

FIG. 1 is a schematic representation showing the study which was setup for the reproducibility of OCTRIMA measurements. A total of 10 healthy eyes were scanned during 4 sessions (E1D1S1, E1D1S2, E2D1S1 and E1D2S1) in 2 consecutive days (D1, D2) by two OCT examiners (E1, E2) and analyzed by two independent experienced graders (G1, G2). The first grader (G1) segmented all the B-scans from all sessions (E1D1S1, E1D1S2, E2D1S1 and E1D2S1) using OCTRIMA. All the B-scans from the first session (E1D1S1) were also segmented by the second grader (G2) and results were used in the intergrader reproducibility analysis. The OCT B-scans from the first session (E1D1S1) were graded twice by one grader (G2) one week after the initial assessment to assess intragrader reproducibility of OCTRIMA.

FIGS. 2A and 2B show photographs of an OCTRIMA screenshot. FIG. 2A Automatic mode showing original raw image (B-scan) with overlaid retinal boundaries labeled. FIG. 2B Manual mode displaying the manual toolbar when the “Start Manual Corrections Tool” button is clicked.

FIGS. 3A to 3F are photographs showing a segmented B-scan (horizontal radial line scan, right eye) before and after applying manual corrections. FIG. 3A: visible segmentation errors such as small peaks, linear offsets, curve offsets and false segmentations. Manual error removal results for: FIG. 3B shows the false segmentation. Note the falsely detected outer boundaries of the RNFL which is often barely visible on the temporal side of the macula, like in this case; FIG. 3C: Small peaks. Note the tiny overshoots or undershoots visible along the boundaries outlined in yellow and cyan, resulting in inaccurate segmentation; FIG. 3D: Foveal center control. A predefined control is also in place for the inner retinal layers in a 1.5 mm diameter zone in the fovea (see results after manual correction), where retinal reflections are minimally visible. The control forces the ILM, the inner and outer side of the GCL+IPL complex, and the outer side of the INL and OPL to be coincident in this region; FIG. 3E: Linear offsets. These are parts of a boundary that form a straight line segment but are incorrectly detected as a peak or an elevated or depressed line segment by the automated algorithm (e.g. see the errors in the outer boundary of the OPL (outlined in green); FIG. 3F: Curved offsets. Curve offset is a term given to the curved portion of a boundary that has not been recognized as a curve, instead, has been incorrectly segmented as an elevated or depressed curve (e.g. see the manual corrections for the inner and outer boundaries of the INL (outlined in yellow and cyan, respectively). The border outlined in blue corresponds to the outer boundary of the choriocapillaries (ChCap) segment that can be extracted manually using the semi-automated approach in OCTRIMA.

FIG. 4 is a photograph showing an OCTRIMA software screenshot showing the quantitative analysis results for a diabetic patient (OD). Thickness maps are shown for the macula (i.e. total retina) and intraretinal layers. The normative data was composed using OCT data from 74 healthy eyes (34±16 years). The nine ETDRS macular regions are: R1—Fovea, R2—Superior Inner Macula, R3—Nasal Inner Macula, R4—Inferior Inner Macula, R5—Temporal Inner Macula, R6—Superior Outer Macula, R7—Nasal Outer Macula, R8—Inferior Outer Macula and R9—Temporal Outer Macula.

FIG. 5A shows the automatic segmentation results obtained for a 6 mm diameter retinal scan at 90 degrees (radial lines protocol) through the fovea for a diabetic patient (51 years old, OD) with mild retinopathy. The boundaries detected are superimposed on the original OCT image, for the abbreviations see the text. Segmentation results were performed using OCTRIMA software. FIG. 5B shows the OCTRIMA thickness maps for the patient data shown in FIG. 5A. The middle panel shows the total retinal thickness map. The lower panel shows the thickness map for the intraretinal layers. An OCTRIMA macular map is divided into nine zones that correspond to the ETDRS regions: fovea within a diameter of 1 mm centered on the foveola; pericentral ring, the circular band from the central 1 mm to 3 mm, divided into four quadrants i.e. superior, inferior, temporal, and nasal; and peripheral ring from 3 mm up to 6 mm, divided into the same quadrants.

FIG. 6A shows the OCTRIMA segmentation results for a patient with neovascular AMD (non aligned raw data) showing the fluid-filled regions outlined. The ONL outer border was used as the outer retinal border in order to compare results with Stratus OCT algorithms (see Table at the bottom). The retinal thickness map along with the ETDRS topographic map including the retinal structure and fluid-filled regions are shown at the right-bottom section. FIG. 6B shows the OCTRIMA segmentation results for an OCT image obtained with the Bioptigen Spectral Domain ophthalmic imaging system. FIG. 6C shows the OCTRIMA segmentation results for an OCT image obtained with the custom developed FD-OCT adapted from our anterior segment OCT system.

FIGS. 7A, 7B are photographs showing a graphical user interface (GUI) screenshot. FIG. 7A: Main program window of the GUI in its first stage of development. FIG. 7B: Scattering coefficient and co-occurrence based classifier results for a consecutive image in the data set after registration.

FIG. 8 shows the averaged scattering coefficient results per scan obtained for the RNFL and the GCL+IPL complex before (NF: non filtering) and after speckle denoising (F: filtering). OCT raw data from normal healthy eyes and eyes with minimal diabetic retinopathy (DR) were used. Higher values are obtained for the scattering coefficient when speckle noise is not removed.

FIG. 9 is a schematic representation of the principles of the extraction algorithm. Note that three macular regions are defined: mid-foveal, mid-perifoveal and mid-parafoveal (see orange dashed insets). A transverse ROI (light blue dashed inset) is selected and an averaged A-scan is obtained. Then the curve-fitting is performed on this resulting averaged A-scan for every segmented layer. Note that in this demonstrative example, the curve-fitting process has been only shown for one of the segmented layers.

FIG. 10 is a schematic diagram of a computer system 1000 for executing a set of instructions that, when executed, can cause the computer system to perform one or more of the methodologies and procedures described above. For example, a computer system 1000 can be implemented to perform the various tasks of the systems 400, 500, 600, 700, 800. In some embodiments, the computer system 1000 operates as a single standalone device. In other embodiments, the computer system 1000 can be connected (e.g., using a network) to other computing devices to perform various tasks in a distributed fashion. In a networked deployment, the computer system 1000 can operate in the capacity of a server or a client developer machine in server-client developer network environment, or as a peer machine in a peer-to-peer (or distributed) network environment.

FIG. 11 shows the comparison of the inner and outer boundary detection between OCTRIMA, RTVue and Stratus (left, from top to bottom) and the GCC boundary detection between RTVue and OCTRIMA (right).

FIG. 12 are graphs showing the comparison of RT (left) and GCC measurements (right) between OCTRIMA and the softwares of the OCT devices.

FIG. 13 shows the Retinal scanning used for evaluating multiple sclerosis.

FIG. 14 shows macular image segmentation using OCTRIMA for evaluating multiple sclerosis.

FIG. 15 shows regional differences between the non-affected eyes of MS patients and healthy eyes.

FIG. 16 shows a schematic overlay of the ETDRS subfields used in an OCT analysis and the mfERG hexagons.

FIG. 17 shows a clinical example of a healthy control, an eye from the DRF, and an eye from the NCRF group with respective structural and functional data.

FIG. 18 shows bar charts of retinal layer thickness results in the central, in the pericentral, and in the peripheral regions.

FIG. 19 schematically shows retinal layer thickness results in the central, in the pericentral, and in the peripheral regions.

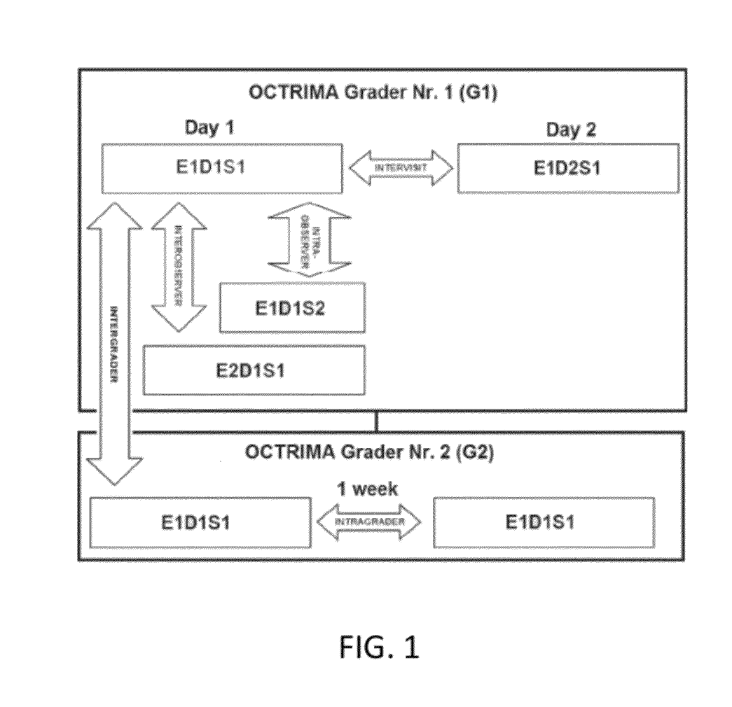

### DETAILED DESCRIPTION

An objective quantitative tool for the early diagnosis and evaluation of treatment of neurodegenerative and ocular diseases and disorders is provided. Optical Coherence Tomography (OCT) performs high resolution imaging of retinal structure non-invasively and in real time. OCT images can either be used to qualitatively assess retinal features and pathologies or to objectively make quantitative measurements. For example, in the case of an ocular disease or disorder, OCT can facilitate decisions on the treatment protocol (surgical or medical) and follow-up of patients, which is especially important in the early stages of diabetic maculopathy when the structural changes are not yet evident with slit-lamp biomicroscopy or angiographically. In another example, the correlation between retinal features and pathologies and neurodegenerative disease state can be utilized with OCT to facilitate decisions on the treatment protocol and follow-up of patients, which is especially important in the early stages of muscular dystrophy, Alzheimer's disease, and Parkinson's disesase.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Throughout this application, the term “about” is used to indicate that a value includes the standard deviation of error for the device or method being employed to determine the value.

The word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is if, X employs A; X employs B; or X employs both A and B, then “X employs A or B” is satisfied under any of the foregoing instances.

Although some aspects of the present invention, such as the collection and analysis of optical coherence tomography data will be described with respect to diabetic retinopathy, this is solely for ease of illustration. The methods described herein are equally applicable to the diagnosis and the characterization of any ocular diseases or disorders, especially in the case of patients with neurodegenerative diseases or disorders.

### Optical Coherence Tomography for Diabetic Retinopathy

Computer based analysis of medical images pertaining to ascertaining retinal structure allows for the diagnosis of retinal diseases and function. Identifying and measuring the various cellular layers of the retina allows a medical expert to access disease states and prescribe therapeutic regimens. In embodiments of the invention, a set of quantitative measures were developed that have been integrated in an image-base analysis software tool specifically designed for OCT images. The system and methods in this invention is significant because it allows for the assessing of optical properties and structure morphology differences between normal healthy subjects and diabetic patients with retinopathy up to ETDRS level 35 and without retinopathy. At the same time, the quantitative measures facilitate the management of progressive changes before and after treatment of DR once a good evidence base for treatment is detected. Moreover, it is also possible to determine which cellular layer(s) of the retinal structure is (are) involved in DR and whether is there any rule concerning which layer(s) get damaged first.

Embodiments of the invention offer many advantages. One of the advantages is that local abnormalities in the structure of the various cellular layers of the retina can be detected using OCT images. This advantage arises from the quantitative measures and methods disclosed herein. Specifically, the computer-aided diagnostic system and methods disclosed herein enable the measurement of total retinal thickness along with the local thickness and optical properties of the intraretinal layers, which could provide a potential improvement in the clinical application of the OCT technology to early detect and diagnose DR. The computer-aided diagnostic system and methods described herein can be used to analyze OCT data from different OCT systems and device configurations. Exemplary OCT systems include, but are not limited to: time domain and spectral domain OCT. In addition, the preceding step of manifest DR may be a neurodegeneration of the retina which seems to be detectable by OCT mapping of the local retinal abnormalities. This aspect of diabetic retinal changes is not yet a part of the common thinking about diabetes, but future studies using the system and methods described herein, elaborating histological and functional changes of the macula in diabetic patients would shed light on this very first step possibly leading to the further sequelae of DR.

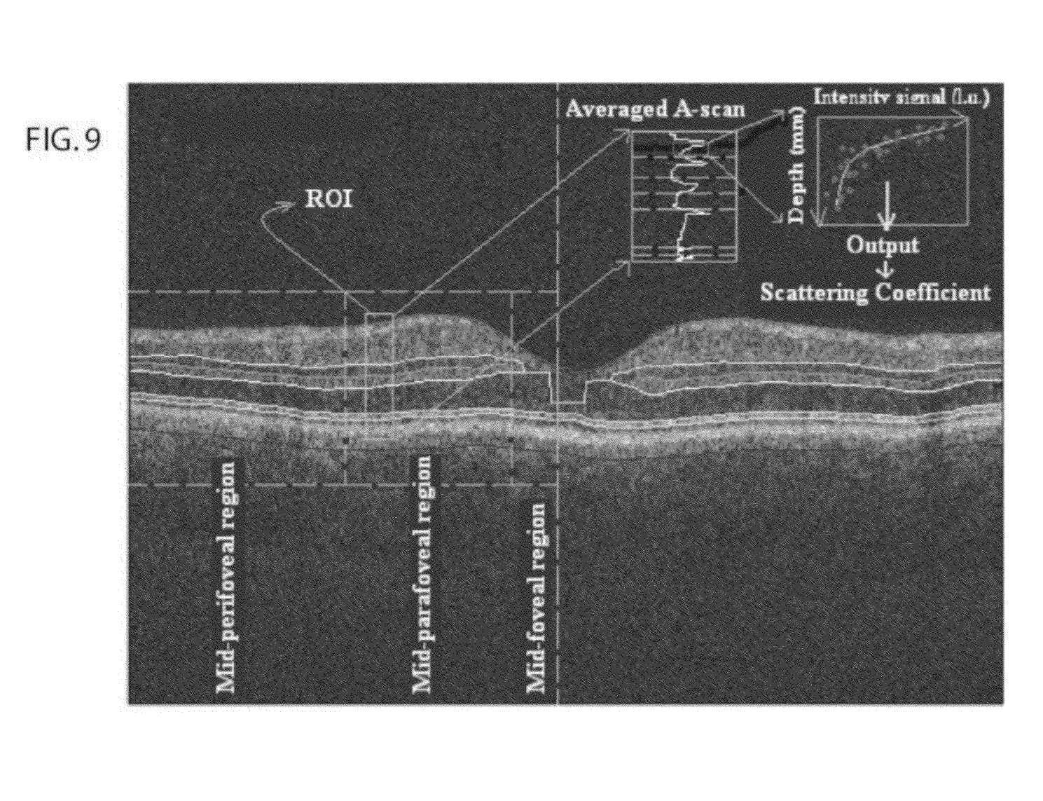

In a preferred embodiment, the present invention provides a method for measuring thickness, extracting optical properties and classifying the tissue of the various cellular layers of the retina of a subject in order to identify local abnormalities in the retinal structure. The extraction of optical properties comprises the local measurement of tissue reflectivity along with the estimation of tissue scattering coefficients from OCT images through mathematical processing (see, for example, FIG. 9). Tissue classification comprises the classification of retinal tissue into one of two possibilities (normal/abnormal) through texture analysis algorithms.

In general, methods according the embodiments of the present invention comprise the steps of comparing the thickness, optical properties and tissue classification of the various cellular layers of the retina of a subject with normal reference data to identify local abnormalities in the retinal structure, wherein the normal reference data is compiled from thickness and optical properties of the various cellular layers of the retina of a population with normal eyes, and wherein data extracted from the subject is abnormal if it meets predetermined comparison criteria.

The normal reference data may be preferably generated from optical coherence tomograms. Reference thickness and reflectance data may be obtained by averaging retinal thickness and reflectance data of the intraretinal layers of a population of healthy individuals with normal eyes. An exemplary reference retinal thickness data that is also known as a thickness map may contain average thickness values of each coordinate on the map and the corresponding standard deviations.

In a preferred embodiment, a reference data or map of the present invention generally may be compiled as follows:

Subjects are assigned to normal group if both eyes had intraocular pressure (IOP) of less than 21 mm Hg, a normal visual field (VF) (defined as having a mean deviation and pattern standard deviation within 95% limits of the normal reference and a glaucoma hemifield test within 97% limits), a normal foveal thickness (mean thickness in the central 1000-μm diameter area) and normal central foveal thickness (mean thickness at the point of intersection of 6 radial scans), a normal-appearing optic nerve head, and a normal nerve fiber layer and normal retina in macular region and if the participant did not have a history of chronic ocular or systemic corticosteroid use.

The step of comparing the retinal thickness map of each intraretinal layer of a subject to a reference map comprises making a comparison of the thickness value at each corresponding region or coordinate of the maps. If the value of the subject's retinal thickness map exceeds the value of the reference map at the corresponding map coordinate by a preset multiple of standard deviation (SD), an abnormal thickness is indicated and it is included in the “abnormal classification”. Selection of this preset “cut-off” threshold depends on the desired confidence level. Preferably, the preset threshold is about 2.33 standard deviations and above (99 percentile). Other useful threshold values include 1.65 standard deviations (95 percentile) or any other values between 1.65 and 2.33 standard deviations. The same methodology is followed to compare the optical properties and tissue classification measures.

In some embodiments, a method of the present invention further comprises a step of identifying an abnormal pattern by finding one or more regions on the subject's intraretinal thickness map whose value is greater or smaller than a predetermined multiple of standard deviations above the mean value at the same location of the normal reference map. The cut-off threshold for identifying the abnormal pattern is preferably 2.60 standard deviations and above (99.5 percentile). Other suitable pattern thresholds include 3.09 standard deviations (99.9 percentile) to 2.33 standard deviations (99 percentile). To identify an abnormal pattern, a method of the present invention may further comprise searching and identifying an abnormal thickness for a specific intraretinal layer by assessing the regional thickness of the particular layer and classifying it as abnormal if it has a thickness greater or smaller than a predetermined thickness. The threshold level for the abnormal region should be lower or higher than that for the reference. 2SDs were used to define cutoffs for the upper and lower levels of normative values. Normal values were applicable for both control subjects and diabetic patients without evidence of DR. Borderline values were suitable for diabetic patients with early signs of retinopathy. Based on the identified abnormal pattern, methods of the present invention may further make diagnosis of early diabetic retinopathy if the abnormal layer fits a predetermined set of criteria:
Criterion 1: Borderline thickness values
Criterion 2: Borderline reflectance values
Criterion 3: Borderline scattering coefficients
Criterion 4: Borderline texture measures

A good predictor of early diabetic retinopathy (PEDR) is implemented by constructing a weighted function resulting from the combination of the above criteria with the use of weight coefficients:

$$PEDR ( {M}_{D} , {M}_{C} ) = min {\sum }_{i = 1}^{N} {w}_{i} \times d ( {M}_{Di} , {M}_{Ci} ) \qquad ( 1 )$$

Where wᵢ are the weight coefficients (wᵢ≧0, i=1 . . . N), d is the distance function and M_{Di}, M_{Ni}, are the measured values (i.e. thickness, reflectance, scattering coefficient, texture classification) for diabetic patients and subjects in the control group, respectively. N is the number of objective functions or criteria considered in the analysis (N=4). PEDR is a natural distance measure for similarity search where w, is the extent to which measure M_{Di} is matched to M_{Ci}.

The weighted function is then used to differentiate diabetic eyes with early signs of retinopathy from both normal eyes and diabetic patients without retinopathy. The PEDR provides a simple and accurate index for early detection and diagnosis of DR. Therefore, OCT would be a valuable additional tool not only in the follow-up of diabetic retinopathy but also in screening programs for DR.

Other suitable criteria are also embodied in embodiments of the invention. For example, three quantitative measures (QM) may be used to identify pathological changes in the retinal structures from a local point of view. Since OCT measures the intensity of light returning from within a sample, then samples having a higher heterogeneity of optical index of refraction have a larger variance. Thus, diseased retina might have multiple strong back reflections resulting in a relatively high OCT signal variance. Then, the normalized standard deviation within a region of interest (ROI) could be defined as the first QM:

$$QM 1 = \frac{\sqrt{\frac{1}{N - 1} \times {\sum }_{{ROI}_{high}}^{ } {\sum }_{{ROI}_{width}}^{{( I ( x , y ) - \overline{I} )}^{2}} }}{( {I}_{max} - {I}_{min} )} \qquad ( 2 )$$

Note that in order to correct the data for variations in the OCT system settings, the standard deviation should be normalized by the maximum and minimum OCT signal present in the OCT image. The number of pixels in the ROI is denoted by N. Moreover, I(x,y) is the OCT signal as a function of x and y locations within the ROI; and Ī is the average OCT signal within the ROI.

Assuming a ROI corresponding to an (n-m) segment in depth and an (r-l) segment in lateral displacement, an integrated backscattered index can be defined as the second QM:

$$QM 2 = \frac{1}{( {x}_{r} - {x}_{l} ) ( {y}_{n} - {y}_{m} )} {\sum }_{j = r}^{l} {\sum }_{i = n}^{m} \vert I ( {x}_{j} , {y}_{i} ) \vert \qquad ( 3 )$$

In order to identify local abnormalities in the diabetic retinal structure, these quantitative measures may be obtained per ROI for every diabetic patient and compared with the QM norm values obtained for subjects in the control group. The criteria can also be adjusted according to future studies on the treatment of diabetic retinopathy or other forms of retinopathy.

Assuming that peak reflectance intensity of the OCT signal changes significantly in layers or ROIs with early retinopathy signs, an early diabetic retinopathy (EDR) index can be defined as the third QM:

$$QM 3 = \frac{NMR}{NRS} \qquad ( 4 )$$

Where NMR is the normalized mean reflectance and NRS is the normalized reflectance of saturation (i.e. the lowest reflectance of the upper 50 pixels on each A-scan line). The normalization procedure compensates for variation in absolute reflectance intensity between images, caused by eye and head movement, tear film quality, pupil size, and laser detector alignment. The QM3 norm per layer and specific predefined ROIs obtained from subjects in the control group is used to calculate the borderline value that determines the EDR index (QM3).

In another preferred embodiment, a computer-aided system designed to extract almost cellular-like pathophysological information from the retinal tissue in vivo is provided. In general, this system comprises a software tool for OCT retinal image analysis (OCTRIMA) which is an interactive, user-friendly stand-alone application for analyzing OCT retinal images. The application essentially provides dual functionality in a single software package by combining image enhancement and speckle denoising of OCT images along with intraretinal segmentation and error correction using direct visual evaluation of the detected boundaries. OCTRIMA is also able to minimize segmentation errors, give quantitative information of intraretinal structures and also facilitates the analysis of other retinal features that may be of diagnostic and prognostic value, such as morphology and reflectivity. A final report is generated and the complete data resulting from the analysis are also available for further analysis. The processing unit may be a general purpose computing unit such as a PC, a workstation, or any other suitable processing unit known in the art. Various software tools may be used to configure the processing unit. For example, the processing unit may be equipped with customized machine codes to perform methods of the present invention to achieve maximum speed. Other software tools that may be used include, but not limited to pre-packed software tools such as MatLab, or application software developed in programming languages such as C, C⁺⁺, JAVA, or any other programming language commonly known in the art.

The OCTRIMA tool is configured to perform an analysis method having the general steps of (i) receiving the tomogram; (ii) image enhancement and speckle denoising; (iii) automatic and semi-automatic segmentation of the intraretinal layers; (iv) computing total retinal and intraretinal thickness maps from the tomogram; (v) computing the intensity at each point of a specific A-scan relative to the value of the highest intensity value along the length of the entire A-scan from the vitreous to the choroids and (vi) identifying which intraretinal layers are abnormal by comparing the thickness map, reflectance data, scattering coefficients and texture measures with the mean and standard deviation of the thickness, reflectance, scattering coefficients and texture measures in similar layers in a population of eyes considered as the control group.

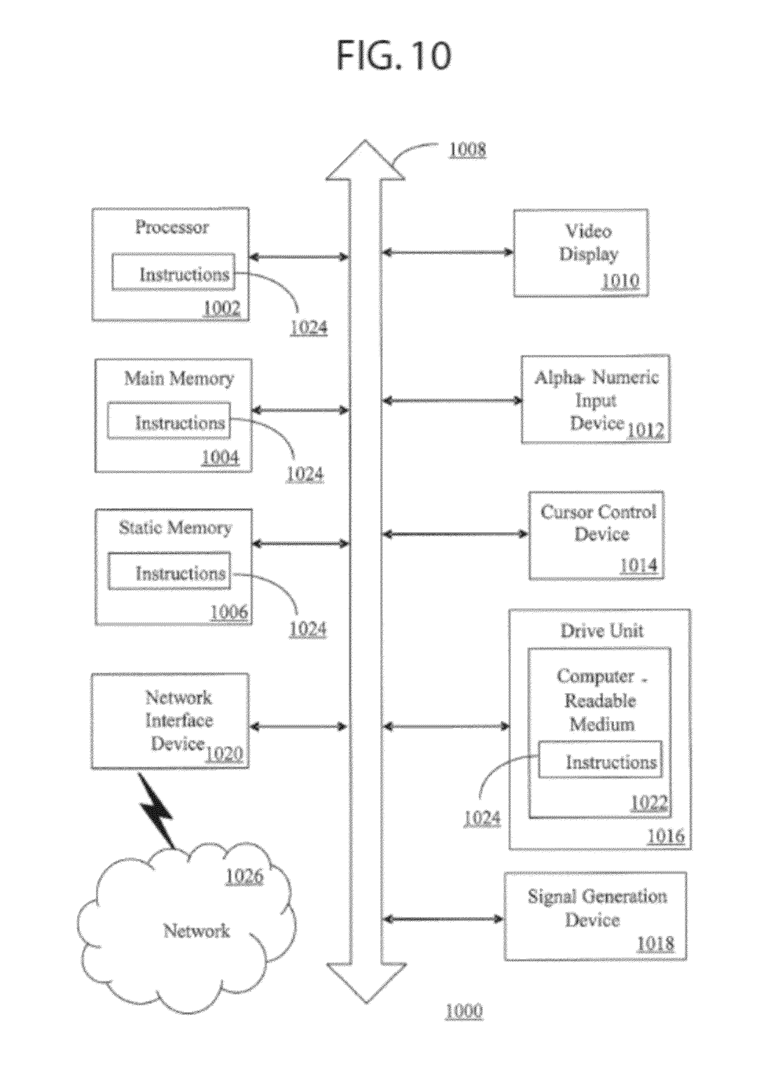

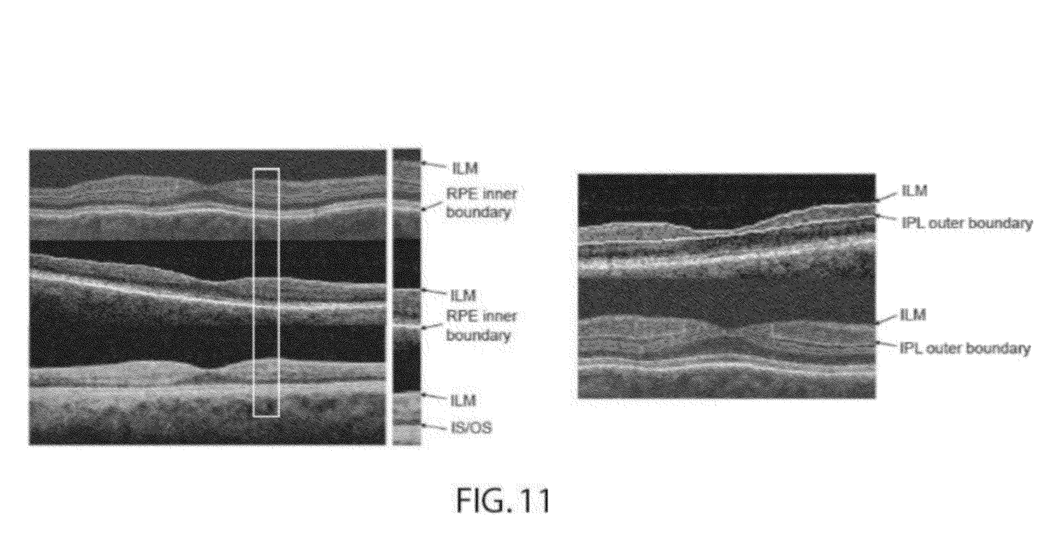

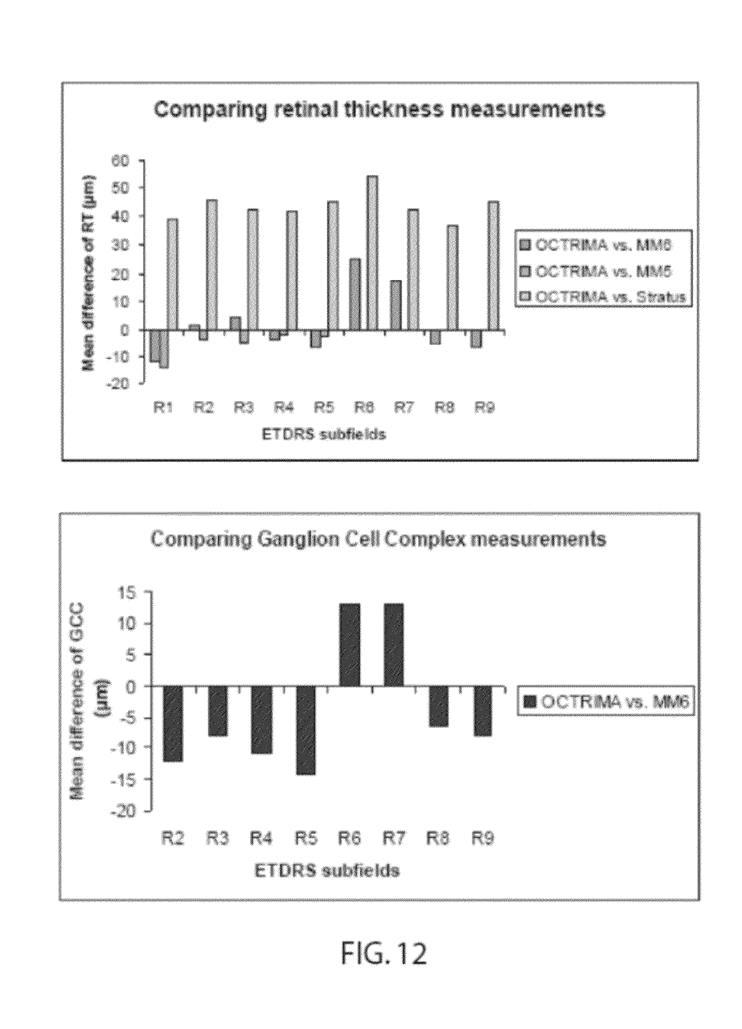

A person skilled in the relevant art will readily recognize that the various aspects of computerization and automation represent different exemplary implementations and utilizations of methods according to the present invention. In a clinical setting, automation and computerization of diagnosis methods and data analysis methods have many advantages. Automation of a diagnosis or analysis method minimizes human involvement, thereby, reduces the chance of human error. In a clinical setting, computerization also enhances data acquisition precision and quality from patient to patient, thereby, allowing a clinic to deliver a more uniform and higher standard of service. For example, FIG. 10 shows a schematic diagram of a computer system 1000 for executing a set of instructions that, when executed, can cause the computer system to perform one or more of the methodologies and procedures described above.

In this illustration, a computer system 1000 can be implemented to perform the various tasks of the systems 400, 500, 600, 700, 800. In some embodiments, the computer system 1000 operates as a single standalone device. In other embodiments, the computer system 1000 can be connected (e.g., using a network) to other computing devices to perform various tasks in a distributed fashion. In a networked deployment, the computer system 1000 can operate in the capacity of a server or a client developer machine in server-client developer network environment, or as a peer machine in a peer-to-peer (or distributed) network environment.

The computer system 1000 can comprise various types of computing systems and devices, including a server computer, a client user computer, a personal computer (PC), a tablet PC, a laptop computer, a desktop computer, a control system, a network router, switch or bridge, or any other device capable of executing a set of instructions (sequential or otherwise) that specifies actions to be taken by that device. It is to be understood that a device of the present disclosure also includes any electronic device that provides voice, video or data communication. Further, while a single computer is illustrated, the phrase “computer system” shall be understood to include any collection of computing devices that individually or jointly execute a set (or multiple sets) of instructions to perform any one or more of the methodologies discussed herein.

The computer system 1000 can include a processor 1002 (such as a central processing unit (CPU), a graphics processing unit (GPU, or both), a main memory 1004 and a static memory 1006, which communicate with each other via a bus 1008. The computer system 1000 can further include a display unit 1010, such as a video display (e.g., a liquid crystal display or LCD), a flat panel, a solid state display, or a cathode ray tube (CRT)). The computer system 1000 can include an input device 1012 (e.g., a keyboard), a cursor control device 1014 (e.g., a mouse), a disk drive unit 1016, a signal generation device 1018 (e.g., a speaker or remote control) and a network interface device 1020.

The disk drive unit 1016 can include a computer-readable storage medium 1022 on which is stored one or more sets of instructions 1024 (e.g., software code) configured to implement one or more of the methodologies, procedures, or functions described herein. The instructions 1024 can also reside, completely or at least partially, within the main memory 1004, the static memory 1006, and/or within the processor 1002 during execution thereof by the computer system 1000. The main memory 1004 and the processor 1002 also can constitute machine-readable media.

Dedicated hardware implementations including, but not limited to, application-specific integrated circuits, programmable logic arrays, and other hardware devices can likewise be constructed to implement the methods described herein. Applications that can include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the exemplary system is applicable to software, firmware, and hardware implementations.

In accordance with various embodiments of the present disclosure, the methods described herein can be stored as software programs in a computer-readable storage medium and can be configured for running on a computer processor. Furthermore, software implementations can include, but are not limited to, distributed processing, component/object distributed processing, parallel processing, virtual machine processing, which can also be constructed to implement the methods described herein.

The present disclosure contemplates a computer-readable storage medium containing instructions 1024 or that receives and executes instructions 1024 from a propagated signal so that a device connected to a network environment 1026 can send or receive voice and/or video data, and that can communicate over the network 1026 using the instructions 1024. The instructions 1024 can further be transmitted or received over a network 1026 via the network interface device 1020.

While the computer-readable storage medium 1022 is shown in an exemplary embodiment to be a single storage medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure.

The term “computer-readable medium” shall accordingly be taken to include, but not be limited to, solid-state memories such as a memory card or other package that houses one or more read-only (non-volatile) memories, random access memories, or other re-writable (volatile) memories; magneto-optical or optical medium such as a disk or tape; as well as carrier wave signals such as a signal embodying computer instructions in a transmission medium; and/or a digital file attachment to e-mail or other self-contained information archive or set of archives considered to be a distribution medium equivalent to a tangible storage medium. Accordingly, the disclosure is considered to include any one or more of a computer-readable medium or a distribution medium, as listed herein and to include recognized equivalents and successor media, in which the software implementations herein are stored.

Although the present specification describes components and functions implemented in the embodiments with reference to particular standards and protocols, the disclosure is not limited to such standards and protocols. Each of the standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same functions are considered equivalents.

In light of the forgoing description of the invention, it should be recognized that the present invention can be realized in hardware, software, or a combination of hardware and software. A method for determining a reference signal according to the present invention can be realized in a centralized fashion in one processing system, or in a distributed fashion where different elements are spread across several interconnected processing systems. Any kind of computer system, or other apparatus adapted for carrying out the methods described herein, is suited. A typical combination of hardware and software could be a general purpose computer processor, with a computer program that, when being loaded and executed, controls the computer processor such that it carries out the methods described herein. Of course, an application specific integrated circuit (ASIC), and/or a field programmable gate array (FPGA) could also be used to achieve a similar result.

Applicants present certain theoretical aspects above that are believed to be accurate that appear to explain observations made regarding embodiments of the present invention. However, embodiments of the present invention may be practiced without the theoretical aspects presented. Moreover, the theoretical aspects are presented with the understanding that Applicants do not seek to be bound by the theory presented.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Given the exemplary embodiments described above, other implementations and modifications not explicitly described are also possible.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

The following examples serve to illustrate the invention without limiting it thereby. It will be understood that variations and modifications can be made without departing from the spirit and scope of the invention.

Embodiments of the invention may be practiced without the theoretical aspects presented. Moreover, the theoretical aspects are presented with the understanding that Applicants do not seek to be bound by the theory presented.

### Example 1

### OCTRIMA: a Novel Tool for Automated Analysis and Interactive Quantitative Evaluation of Clinical Stratus OCT Data

Software Implementation: OCTRIMA was developed using the Matlab graphical user interface design environment (GUIDE) tool that allows interactive design of a graphical window along with variables and commands linked to it (The Mathworks, Natick, Mass.). Specifically, OCTRIMA is a research software application package that integrates the automated and semi-automated segmentation algorithm along with the manual correction tool into a user-friendly graphical user interface (GUI). The OCTRIMA software originally written in Matlab code is converted into C and requires the MatLab Component Runtime (MCR), which must also be installed on the end-user's computer. It also requires a PC with the WINDOWS™ operating system installed (Microsoft Corp., Redmond, Wash.). The application essentially provides dual functionality in a single software package by combining image enhancement and speckle denoising of Stratus OCT images along with intraretinal segmentation and error correction using direct visual evaluation of the detected boundaries. Moreover, the software has the capability to perform calculations based on measured values of corrected thickness and reflectance of the various cellular layers of the retina and the whole macula.

Software Description: The design of OCTRIMA is partially based on the principles of encapsulation, which is the process of hiding all of the details of an object that do not contribute to its essential characteristics; typically the structure of an object is hidden, as well as the implementation of its methods. This is beneficial to both, the user and the developer, since the user does not need to know the internal architecture and the functionality of an object and the developer can improve implementation details without altering the interface. The user interface was mainly organized into panels that group GUI components and make the GUI easier to understand by visually grouping related controls. The GUI guides the user in an organized manner by activating only relevant functions and disabling other functions at any given time, achieved by effectively using the Matlab handles structure.

Data Import: The input of the OCTRIMA software simply consists of two types of data files:
the data file with the patient information (i.e. a text file exported from the Stratus OCT system)
the raw scan data (i.e. the image file).

OCTRIMA facilitates the analysis of OCT images from two OCT scanning protocols: regular high-resolution and fast low-density mode. The built-in export function on the Stratus OCT system facilitates that a set of 6 files for each B-scan can be exported to a portable storage device:

Filename.txt (Patient information in ASCII text format)
Filename.raw (Raw scan or B-scan data in binary format)
Filename.bmp (Processed scan image in bitmap format)
Filename.vi1 (Fundus video image in bitmap format)
Filename.spd (Raw scan data in ASCII text format)
Filename.rnf (RNFL Thickness result in ASCII text format)

As previously mentioned, OCTRIMA only uses two types of data files: “Filename.txt” and “Filename.raw”.

Detailed Menu Design

The file, view, data and help menus provide various options for:
file management
image data and segmentation results viewing
image processing and quantitative analysis
generating and exporting the results in various format
comprehensive visual help.

As a general rule, the user has to choose an option from a pre-defined menu in OCTRIMA. This may include opening or loading image files and other data files to display an image on the screen or to perform calculations using information from the data file. For example, the various options in the file, view, data and help menus are available to the user and certain functions get activated during particular operations so that the user is well guided to choose a valid option from the limited active set of functions. Additionally, the user can choose to accept or reject the result generated by the program. Different interaction techniques are adopted to implement the choice from pre-defined options, such as push buttons, check boxes, text boxes, list boxes, popup menus and radio buttons.

File menu: The File menu provides options for OCT file operations such as loading, saving and viewing raw data (i.e. B-scans); printing options and the exit function to quit the GUI.

OCT scan files can be loaded in two different ways: 1. A new OCT B-scan that is yet to be segmented must be opened using the “New Segmentation Analysis” option, for preprocessing and segmenting the image. 2. A formerly segmented OCT B-scan that has corresponding saved segmentation data can be loaded using the second option for review. Once the scan to be reviewed is opened, the GUI automatically prompts the user to load the saved segmentation data file.

Multiple OCT B-scans can be loaded at once and viewed one at a time by using the review option in the file menu. In addition, the automated and semi-automated segmentation algorithm as well as the manual correction tool are able to process and modify the same OCT B-scan. Thus avoiding redundant steps such as loading the OCT B-scan file twice or saving the same data multiple times.

View menu: The user may choose to view the coherence enhanced image or the raw image from the View menu once the image has been preprocessed (see the demos in the video attached). The coherence enhanced image is obtained as result of filtering the image and also provides an additional visual tool to evaluate the quality of the corrections during the manual correction mode. The segmented layers can be viewed in an aligned manner with respect to the ONL, which gives the user an organized view of the OCT B-scan as well as the detected boundaries of the various cellular layers of the retina. The user is given the option to delete all the detected boundaries, if necessary, and restart the segmentation function or clear the segmentation for the RNFL on the left side or right side, if required. The delete operations as well as aligning of layers are each accompanied by an undo option in this menu.

Data menu: The Data menu consist of functions that use custom built algorithms developed for generating the report for quantitative analysis, exporting the numerical results (i.e. thickness and reflectance data) to a MS Excel spreadsheet and generating topographic and fractional loss maps for the retinal thickness of the overall macula and each intraretinal layer. In addition, a final report in PDF format can be also generated.

Help menu: The Help menu allows the user to view the OCTRIMA user guide, which explains in detail the functionalities of the software.

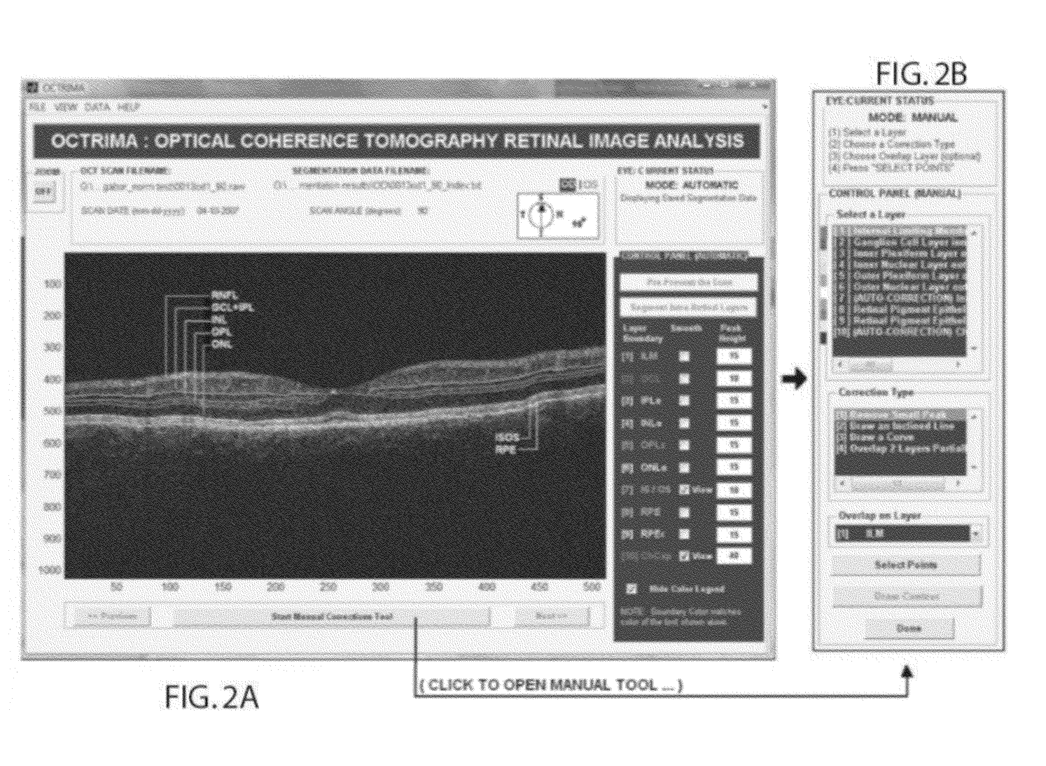

OCTRIMA in automated mode: FIGS. 2A and 2B show the OCTRIMA startup screen in the automated mode. The general information panel displays the name of the displayed OCT B-scan and its saved segmentation results as well as important subject and OCT B-scan information such as left eye (OS) or right eye (OD), scan date, the scan angle orientation (in degrees) and its pictorial representation. The subject and OCT B-scan information is automatically extracted from the text file which is exported along with the raw scan file from the Stratus OCT system. The detailed screen design for OCTRIMA in automated mode is as follows:
Main axes object: Displays the OCT B-scan data on the image grid.
The “Status Panel” as well as message dialogue windows indicates the current status or prompts the user to execute the next processing step. The goal is to keep the user always updated about any process completions or results generated.
The “Control Panel” in automatic mode facilitates the following functionalities: 1. Pre-processing of OCT raw data. The filtering of the speckle noise is performed during the preprocessing step. The pre-processing algorithm parameters are a set of invariant numerical values that already have been optimized and hence it is encapsulated and invisible to the user. More details of the denoising process can be found in Salinas et al. (IEEE Transaction on Medical Imaging, Vol. 26, Issue 06, pp. 761-71, 2007). 2. Automated segmentation of the various cellular layers of the retina. Segmentation is achieved by finding peaks on each sampling line using the structure coherence matrix. A total of eight intraretinal boundaries are automatically detected while the outer boundary of the IS/OS and a Choriocapillaris section are assumed at fixed distances using anatomical knowledge. Thus, a total of 10 boundaries are extracted. More details of the segmentation process can be found in Cabrera et al. (Opt. Express 13, 10200-16 (2005)). 3. Semi-automatic correction of discontinuities in each detected boundary after automated segmentation. This correction is performed by activating the following functions:
“Smooth” Check boxes: Use input peak heights to correct error peaks in each corresponding boundary (see the Control Panel section in FIG. 2A). This function is used to correct discontinuous segments in a retinal boundary. A discontinuity is in place when two transversely adjacent pixels in the boundary are separated in longitudinal distance by more than 20 pixels. For example, spikes are typical discontinuities observed in OCT B-scans after automated segmentation. These discontinuities are corrected by approximating the boundary in the region of the discontinuity using linear interpolation (i.e. a straight line is drawn between the discontinuities in the region).
“Peak Height” Text boxes: Accept user input of peak heights for each boundary (see the Control Panel section in FIG. 2A). This functionality is used to select the discontinuous segments in each corresponding boundary that need to be corrected after automatic segmentation. The selection requires the information about the peak's heights the user wants to correct. Accordingly, the user has the choice to select the peaks or discontinuities per boundary that need to be corrected using the height information.
Zoom ON/OFF push button: Used for zooming in or out.

OCTRIMA in Manual Correction Mode

Segmentation of the object of interest is considered a difficult step in the analysis of medical images. Fully automatic methods sometimes fail, producing incorrect results and requiring the intervention of a human operator. This is often true in ophthalmic applications such as OCT where image segmentation is particularly difficult due to restrictions imposed by image acquisition, ocular pathology and biological variation. Consequently, the intervention of a human operator is often needed to correct the segmentation result manually. Strategies that allow a trained human expert to correct segmentation errors may provide a suitable mechanism for increasing the precision of retinal measurements for monitoring patients with macular disease, particularly in clinical trials.

Computer aided manual correction of OCT segmentation may be useful for correcting thickness measurements in cases with errors of automated retinal boundary detection and, may also be useful for quantitative analysis of clinically relevant features, such as the volume of subretinal fluid and intraretinal fluid-filled regions. It is well known that the lines drawn by the detection algorithms in the current commercial Stratus OCT system are frequently and dramatically erroneously drawn, which can lead to inaccurate measurements of retinal thickness. Thus, there is a need for developing efficient, user-friendly software tools that will supplement fairly accurate automated boundary detection algorithms to generate more precise segmentation of the various cellular layers of the retina. For example, an interactive procedure could be activated, by means of which the user edits the segmentation directly or provides extra information to reconfigure the computational part. If the result generated by the computational part is wrong, the user can correct it directly using a manual editor.

In OCTRIMA, the manual corrections tool is initialized by clicking the push button located below the main figure in the automated mode (see FIG. 2A). The button to start the manual corrections tool, when clicked, is programmed to display the manual control panel and hide the automated control panel while maintaining the current state of the GUI. The menu options and scan information panel remain unchanged while the options for layer selection and corrections are displayed in the manual control panel (see FIG. 2B). The current status panel indicates the general flow of steps while using the manual tool. Opening a new OCT B-scan file or clicking the “Done” button causes the GUI to switch back to automated mode primarily by changing the control panel display.

The detailed screen design for OCTRIMA in manual correction mode is as follows:
Main axes object: Displays the OCT B-scan data on the image grid
“Previous” and “Next” Push buttons: Allow navigation between multiple scans for any specific subject
Zoom ON/OFF push button: Used for zooming in or out.
The control panel in manual mode facilitates the different manual corrections: 1. “Select a Layer” List box: Choose a layer to correct errors, if any. Semi-automated boundaries, such as the IS/OS junction and the Choriocapillaris cannot be manually corrected in this OCTRIMA software version since they are assumed to be located at constant distances from the ONL and the RPE outer boundary respectively (see FIGS. 2A, 2B). 2. “Correction type” List box: Choose a correction type. 3. “Overlap on Boundary” Popup menu: Select an overlapping boundary. 4. “Draw Contour” Push button: Trace the boundary of specific regions in pathological cases such as macular holes, subretinal fluid and macular cysts using a customized curve plotting algorithm. This functionality is also used in cases with a complete absence of a retinal boundary in some portion of the image. This condition would usually occur with 1) blood vessels or hard exudates which caused shadowing and loss of the reflections from the RPE and choroid, or 2) patient blinks which caused a complete loss of signal during the blink.
“Done” Push button: Return to automatic mode.

Overview of the manual corrective functions: Various functions were implemented to assist the user in correcting retinal segmentation errors resulting from the automated and semi-automated segmentation process. All the functions and algorithms used in the manual correction process display the delineation of the retinal boundaries on the screen, enabling the user to instantly evaluate its location on the image grid. Different visualization schemes are adopted, such as to show the detected layers as well as the corrections in color for better discrimination from the grey image in the background.

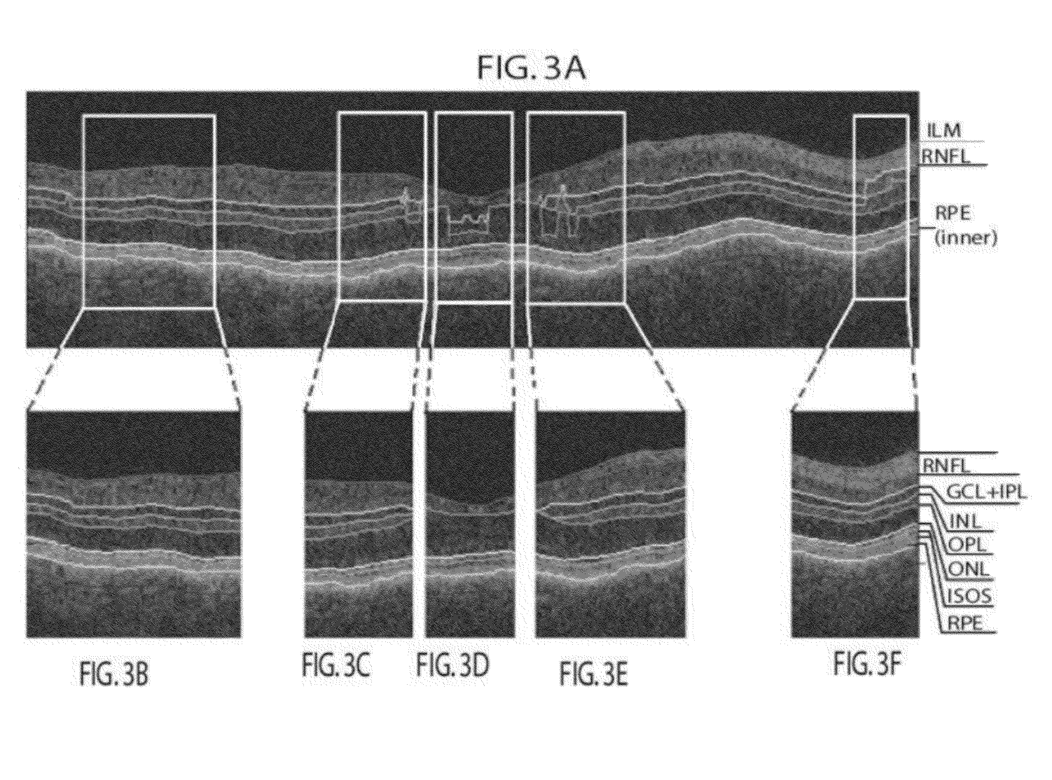

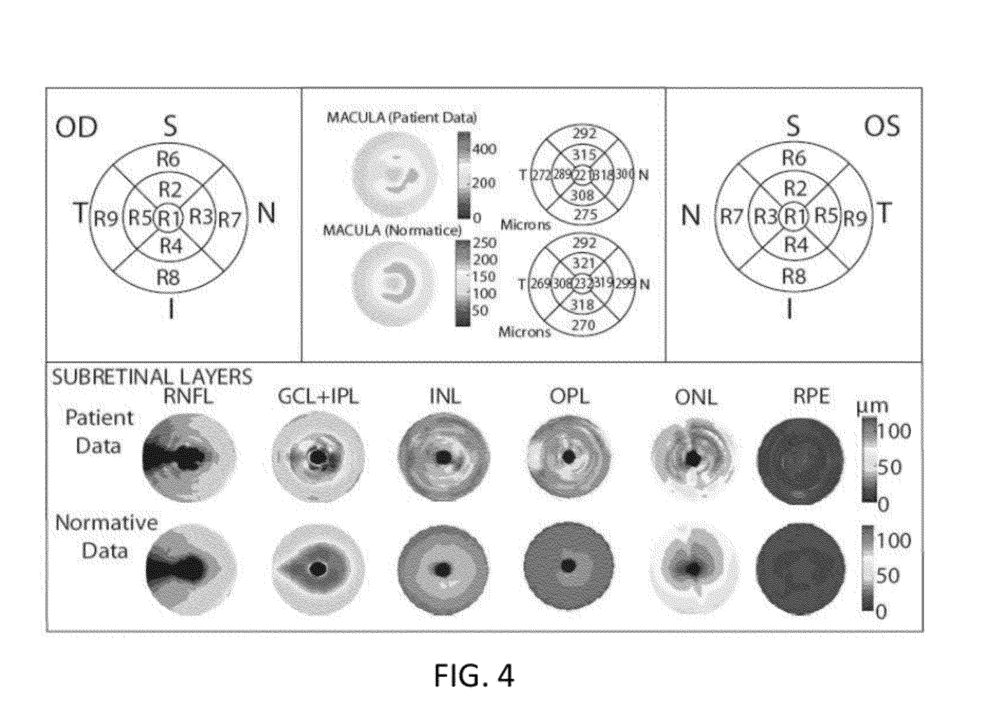

These errors are mainly due to both the presence of high reflectivity regions in the inner retina and loss of retinal structure information in local regions along the retinal cross-section as visualized by the commercial OCT system. The errors are classified as: (a) False Segmentation—It refers to the falsely detected inner and/or outer boundaries of an intraretinal layer. This particular error is most commonly found during the RNFL detection. This happens because the reflectance of the RNFL is highly directional and depends strongly on the angles of illumination and viewing. Specifically, there are certain cases in which the true anatomical thickness of the RNFL layer (or some regions of the RNFL layer) might be negligible. In other cases, one side of the RNFL layer is completely invisible in the OCT image, like for example in the horizontal B-scans. In such cases, a correction is required to overlap the inner and outer boundaries of the RNFL layer in the regions of negligible thickness (see, FIGS. 3C and 3D). However, sometimes the boundary detection algorithm fails in such specific cases when localized bright spots of high intensity appear on some regions of the RNFL layer; and falsely displays the outer boundary of the RNFL layer as a result of the peak search algorithm which looks for zero crossings in the structure. Hence, the RNFL outer boundary must be manually corrected to appear overlap the inner boundary in the invisible part of the layer. As can be seen in FIG. 3B, the ILM boundary on the inner side of the RNFL is detected but no boundary is detected on the outer left side since the RNFL is not visible on this side for this particular scan, whereas the RNFL is bright and clearly visible on the right side of the scan (see FIGS. 3A and 3B).

Additionally, a predefined control is also in place for the inner retinal layers in a 1.5 mm diameter zone in the fovea, where retinal reflections are minimally visible. The control forces the ILM, the inner and outer side of the GCL+IPL complex, and the outer side of the INL and OPL to be coincident in this region (see FIG. 3A). Sometimes, small peaks appear at the periphery of this controlled foveal region. In such a case, it appears that the coincident layers deviate from the true foveal visible boundary and need to be corrected so that they overlap in the periphery of the controlled foveal region. Thus, the overlap function of the manual correction software tool is useful to rectify the segmentation at the fovea (see FIG. 3D).

Small Peaks: Small peaks refer to tiny overshoots or undershoots visible along an intraretinal boundary, resulting in inaccurate segmentation (e.g. FIG. 3A shows small peaks in the boundaries outlined in yellow and cyan). The user's visual information about the peak height (in pixels) is used to remove the peaks after the automated segmentation results are obtained. However, the peak height once selected by the user is assumed the same along the entire length of each independent layer. As a result, not all peaks along the boundary can be removed at once. Thus, additional manual interaction is required in order to completely correct the small peak errors. The “small peak” corrective function of the manual correction software tool removes the overshoots or undershoots in the individual boundaries. For example, FIG. 3C shows the manually corrected outer boundary of the IPL (outlined in yellow). In this case, the user is required to manually select the starting and ending points of the peak and these two points are joined to remove the peak.

Linear Offsets: These are parts of a boundary that form a straight line segment but are incorrectly detected as a peak or an elevated or depressed line segment by the automated segmentation algorithm. To resolve this class of errors, the user has to manually select two points to draw a straight line segment on the specific boundary containing the offset. For example, a straight line segment was manually drawn to correct the linear offset in the outer boundary of the OPL (see the boundary outlined in green in FIG. 3E).

Curve Offsets: Curve offset is a term given to the curved portion of a boundary that has not been recognized as a curve, instead, has been incorrectly segmented as an elevated or depressed curve. For example, FIG. 3F shows the manual corrections for the inner and outer boundaries of the INL (outlined in yellow and cyan, respectively) which had segmentation errors as a result of curve offsets (see FIG. 3A). The curve offsets have been rectified using a function based on a customized contour model which was originally introduced to identify non-convex shapes in OCT images. This function allows the user to select multiple closely spaced points that will be joined to trace a curve and remove the offset. FIG. 3F shows the result of the curve plotting function applied to correct the inner and outer boundaries of the INL (outlined in yellow and cyan, respectively).

Manual detection of visible convex shaped structures: The manual correction software tool is designed to overcome the limitations of the automated and semi-automated segmentation algorithm, not only in terms of error correction in detected intraretinal boundaries but also to allow the user to trace the internal boundary of visible non-convex shaped structures such as intraretinal and subretinal fluid-filled regions, if present and visible on the OCT B-scan.

Data export: All the analyzed results can be saved as tab delimited text files for user's convenience. These results are also mapped to specific cells in a Microsoft Excel template. Once the data is loaded in the template, the file is saved with a user specified filename. The template consists of four data analysis fields: 1) analysis per scan, 2) analysis per region; 3) analysis of the overall macula per scan, and 4) analysis of the overall macula per region. The output data includes three main quantitative measures: thickness, volume and reflectance. Additionally, topographic maps of the extracted thickness for the overall macula and each intraretinal layer can be created by selecting the “Generate Topographic Maps” option under the Data menu. These maps are obtained according to the standards set by the Early Treatment Diabetic Retinopathy Study (ETDRS) similarly to the Stratus OCT analysis software and can be easily exported to a PDF document along with the numerical results in tabulated format. A topographic map of the thickness fractional loss in percent is also available. This particular map provides the deviation from the norm. The OCTRIMA's norm was obtained from 74 healthy eyes (34±16 years).

It is noted that the analysis of the overall macula per scan and per region is based on mean values of thickness measured between the ILM and the inner boundary of the RPE layer, which follows the true anatomical position of the inner and outer border of the retina. Moreover, once the intraretinal layers are automatically segmented, the relative reflectance and thickness of these layers at the individual points (i.e. at each of the 512 A-scans) are averaged to yield a mean “raw” measurement of thickness and reflectance per layer. Since the absolute reflectivity can vary according to a wide variety of factors, such as media opacity or scan technique, each reflectivity value is a percentage of the local maximum. In this way, it is possible to compare different scans in the same patient or subject or even among different patients, different operators or OCT machines.

Additional OCTRIMA-based measures: OCTRIMA also offers objective and intuitive additional functions for evaluating and comparing the efficacy of different therapeutic modalities. Since normative data for OCT analysis are crucial to compare various treatment strategies, OCTRIMA facilitates normative data and also allows the user to create a new norm. Specifically, the OCTRIMA's norm is based on data from 74 healthy subjects (34±16 years).

In addition, OCTRIMA provides a standardized method for reporting changes in thickness as a percentage of total possible change based on normative OCT data. The “Calculate standardized thickness change” function calculates the total percentage improvement observed in the patient using the segmentation results before and after treatment. These two OCTRIMA-based measures can be found under the Data menu.

### Example 2

### Early Detection of Retinal Thickness Changes in Diabetes Using Optical Coherence Tomography

The purpose of this study was to explore the ability of intraretinal layer segmentation to locally detect early retinal changes in diabetic patients using OCT, and determine whether OCT can be used to understand the early histological changes of the macula in diabetes by comparing the thickness of the various cellular layers of the retina in diabetic patients who have no retinopathy with the thickness in patients with minimal DR.

Materials and Methods

Subjects: A total of 50 eyes of 38 patients with diabetes mellitus (DM) with no or minimal diabetic retinopathy (MDR) (39 eyes no DR [DM; 36±10 years] and 11 eyes with minimal DR [MDR; 61±20]) on biomicroscopy were included in this study. Glycosylated hemoglobin (HbAlc) level for DM patients was 7±1 (mean±SD); and the presumed duration of DM (from the time of diagnosis to the time of examination) was 22±8 years. DM patients underwent a complete ophthalmologic examination and fundus photography. The inclusion criteria considered diabetic patients with no DR or with the presence of at least one microaneurysm or hemorrhage in the central retina but no other diabetic lesions (i.e. minimal retinopathy), but in all cases without clinical signs of macular edema (CSME). Exclusion criteria were the presence of proliferative disease, CSME, anatomic abnormalities and media opacities that could distort macular architecture, such as vitreoretinal traction, cataract and epiretinal membranes. In addition, patients with visual acuity less than 20/25 and with previous diagnosis of glaucoma, uveitis, or retinal disease were excluded from the study. Written informed consent was obtained from all participants. Procedures followed the tenets of the Declaration of Helsinki, and were approved by the institutional ethics committee.

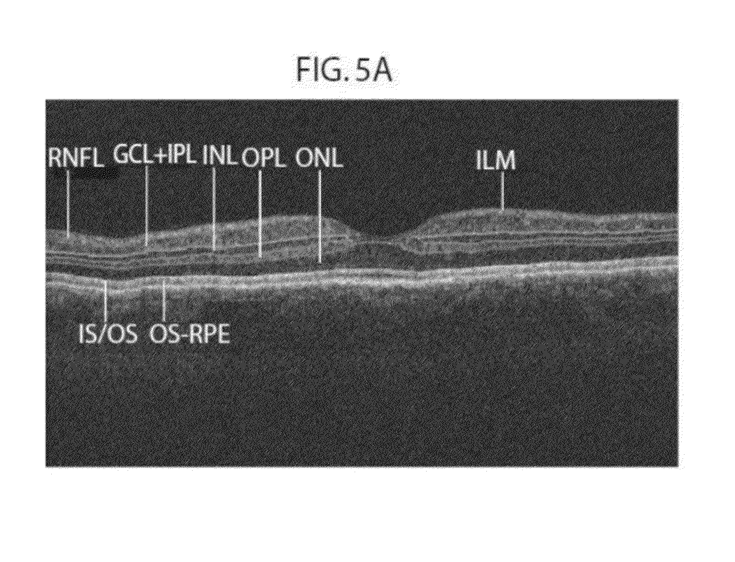

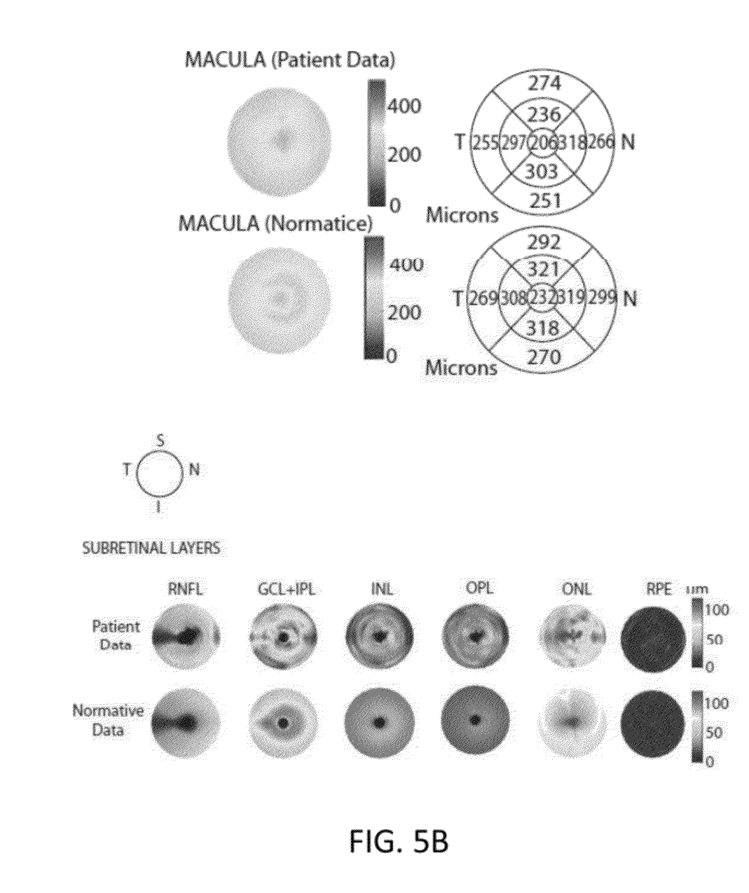

Optical Coherence Tomography imaging and data analysis: For imaging purposes the commercially available Stratus OCT unit (software version 4.0; Carl Zeiss Meditec, Inc., Dublin, Calif.) was used. This imaging system is based on the principle of optical low-coherence interferometry that measures the echo time delay and intensity of backscattered light and thus resolves the position of reflective or optical backscattering sites within a tissue sample. OCT is a fiber-optic based, non-contact and non-invasive imaging system with a high resolution of <10 μm which is one to two orders of magnitude finer than standard ophthalmic ultrasound. The radial lines protocol was used for each subject. The OCT raw data were exported for automatic/semiautomatic subanalysis and quantification of intraretinal layers using a custom-built OCT retinal image analysis software (OCTRIMA) written in Matlab 7.4 (MathWorks, Natick, Massachussetts). OCTRIMA is a powerful computer-aided system designed to facilitate viewing and automatic/semiautomatic OCT retinal image analysis (Cabrera Fernandez D, et al., Invest. Ophthalmol. Vis. Sci. 49, 2008; pp. 2751). The application provides dual functionality in a single software package by combining image enhancement and speckle denoising of Stratus OCT images along with intraretinal segmentation and error correction using direct visual evaluation of the detected boundaries. Moreover, the software has the capability to provide quantitative analysis based on measured values of corrected thickness, volume and reflectance of the various cellular layers of the retina. A total of seven intraretinal layers can be extracted using OCTRIMA, namely, the retinal nerve fiber layer (RNFL), the ganglion cell layer along with the inner plexiform layer (GCL+IPL), the inner nuclear layer (INL), the outer plexiform layer (OPL), the outer nuclear layer (ONL), the photoreceptor inner/outer segment (IS/OS) junction; and the photoreceptor outer segment/retinal pigment epithelium (OS/RPE) junction (see FIG. 5A). Since the border between GCL and IPL could not be differentiated on most scans, the combined thickness was measured as GCL+IPL. In addition, topographic maps of the extracted thickness in 9 ETDRS (Early Treatment Diabetic Retinopathy Study) areas for the overall macula and each intraretinal layer can be obtained similarly to the Stratus OCT analysis software (see FIG. 5B). ETDRS areas include a central 1-mm disc, representing the foveal area, and inner and outer rings of 3 and 6 mm, respectively. The inner and outer rings are divided into four quadrants: superior, nasal, inferior, and temporal.

Stratus OCT uses segmentation algorithms to mark the ILM as the inner boundary and the photoreceptor inner segment/outer segment (IS/OS) junction as the outer boundary. Particularly, the Stratus OCT system images the outer retinal layers (RPE-photoreceptor complex) as two hyperreflective bands: 1) the photoreceptor inner/outer segment junction and 2) RPE-choriocapillaris complex. The segmentation software of the Stratus OCT system uses the anterior border of the most inner hyperreflective band (i.e. photoreceptor inner segment/outer segment junction) as the border of the outer retina for calculating the total retinal thickness. OCTRIMA detects the outer retinal boundary as the anterior border of the second hyperreflective band, which is thought to represent the tip of the cone outer segment in the fovea. Thus, OCTRIMA calculates the total retinal thickness as the distance between the vitreoretinal interface (ILM) and the anterior boundary of the second hyperreflective band corresponding to the OS/RPE junction (see FIG. 5A).

After each B-scan was denoised, the inner and outer borders of the retinal structure were identified between the ILM and OS/RPE junction (inner boundary); and a total of seven intraretinal layers were extracted using OCTRIMA. All scans in the study had signal strength of 9 or 10. Although OCTRIMA is also able to analyze images from SDOCT devices, the algorithm used in this study was optimized for Stratus OCT images. Algorithm performance was visually evaluated to detect algorithm errors. Criteria for algorithm error included evident disruption of the detected boundary (e.g. small peaks, linear and curve offsets), and/or detected boundary jumping to and from different anatomical structures (i.e. false segmentation). The average number of manual corrections needed per scan was three.

Statistical Analysis: Mean values of retinal thickness of the macula and intraretinal layers in the central, pericentral and peripheral macular regions in the diabetic patients with no DR were compared with those in patients with MDR. Regional total retinal thickness was also recorded and compared for the DM and MDR groups. Comparisons between groups were made using Mann-Whitney U test. Because of the number of statistical comparisons made in the study, a modified p value of <0.001 was considered statistically significant. Statistical analyses were performed using Statistica 8.1 (Statsoft Inc., Tusla, Okla.)

Results

Fundus photography showed either no abnormalities or only few microaneurysms in the posterior pole in DM patients. It was confirmed that the measured local thickness of the intraretinal layers was consistent with known retinal anatomy for the diabetic patients. For example, in the diabetic patients with no DR, the GCL+IPL showed maxima in thickness just outside the fovea, while the RNFL increased in thickness between the foveal center and the optic disc. In addition, these layers were decreased in thickness in patients with MDR. Table 1 shows the mean total macular thickness and standard deviation for each study group as measured by OCTRIMA for each ETDRS region separately. A decrease in average total macular thickness was measured with the OCTRIMA software in all areas (R1-R9) for eyes with minimal DR.

Mean intraretinal layer thickness values and total macular thickness (μm) that showed a statistically significant difference (p<0.001) between diabetic eyes with no DR and eyes with MDR are shown in Table 2. When comparing eyes with MDR to diabetic eyes with no DR, a reduced RNFL thickness was found in the pericentral and peripheral macular regions, and reduced thickness of the GCL+IPL in the pericentral region of the macula (p<0.001). Particularly, a 33% and 21% decrease in RNFL was found in the pericentral and peripheral macular regions, respectively, while the GCL+IPL decreased by 13% only in the pericentral region. Total macular thickness was also reduced in the pericentral and peripheral region of the macula (see Table 2). The central part of the macula (mainly corresponding to the fovea) was not affected by early diabetes (see Tables 1 and 2). RPE thickness of the central region may be somewhat affected in the MDR group when compared with the DM group, however this difference did not reach the significance level set in the study.

Discussion

A custom-built algorithm (OCTRIMA) was used to measure the local thickness of the intraretinal layers seen in Stratus OCT images of patients with no and minimal DR. The main interest of this study was in the thickness differences between diabetic patients with no and minimal DR. In particular, the question was whether it was feasible to measure early changes of the macula in diabetes using OCT. In general, the results are encouraging. The present study demonstrates that a software algorithm designed to automatically segment and quantify intraretinal layers of clinical interest on two dimensional OCT images is feasible for locally detecting early retinal changes in diabetic patients using OCT.

In this study it was found that the RNFL and GCL+IPL complex was thinner in DM and MDR eyes than in normal healthy eyes. However, a different assessment was performed in this study by comparing thickness measurements of diabetic eyes without the presence of retinopathy to eyes with minimal diabetic retinopathy. Particularly, it was found that local measurement of the retinal thickness by OCT may provide useful information about the changes of the intraretinal layers in diabetic patients. These results evidence that the RNFL and GCL+IPL complex are more susceptible to initial damage when comparing MDR with DM eyes. This may reflect neurodegenerative changes in the diabetic retina. The trend observed for the thickness of the RNFL and GCL+IPL in MDR eyes might be associated with pathological metabolic changes in the retina. These findings also have possible implications for early detection of macular damage in diabetes. Because the macular region is rich in retinal ganglion cells, it could be that diabetic damage of this central region might occur early in the disease process. In fact, animal models of DR show significant loss of macular ganglion cells.

The strength of this study compared to other studies using Stratus OCT data is the OCTRIMA subanalysis and quantification of the local variations of the intraretinal layers. However, a larger study to evaluate these findings on a greater scale and a more homogeneous data set is required in the future. Despite the worse resolution and scanning time of Stratus OCT systems, comparable thickness measurement differences could be obtained with the recent introduced SDOCT devices. Algorithms have been proposed for segmenting the various cellular layers of the retina and analyzing images from any SDOCT device. However, it is possible to obtain local thickness measurements of the intraretinal layers observed in SDOCT images using OCTRIMA. In summary, the results herein, support this hypothesis and also sustain the view of neurodegeneration in diabetes in the early stage of DR which seems to involve the ganglion cells and cells of the inner plexiform layers mostly, leading to reduced total retinal thickness.

Conclusions

The in vivo human data, may underline the findings of neural apoptosis due to diabetes and may also support the view of diabetic retinopathy as an—at least partly—neurodegenerative disease. This study comparing thickness measurements between diabetic eyes with no DR to MDR eyes demonstrates that local measurement of the retinal thickness by OCT appears to be a proper index for early DR detection and neuroprotection. The local changes in the retinal structure of the diabetic retina could be helpful in finding a surrogate for following development of retinopathy affecting vision. Moreover, it may also be possible to determine which cellular layer(s) of the retinal structure is (are) involved in diabetic retinopathy and whether there is any rule concerning which layer(s) will get damaged first. A larger study will be conducted to evaluate the present findings on a greater scale. In addition, intraretinal layer segmentation may eventually be useful in testing new drugs once it could be demonstrated that changes in the diabetic retina could be used as surrogates for subsequent progress of retinopathy inducing visual defects.

Intraretinal layer quantification on OCT images may greatly help the understanding of macular pathophysiology in health and disease and may soon become a daily diagnostic tool of the comprehensive ophthalmologist with the future development of both OCT hardware and software.

As this study revealed, the preceding step of manifest DR may be a neurodegeneration of the retina which seems to be detectable in vivo by OCT mapping of the local retinal abnormalities and corresponds to previous experimental results. This aspect of diabetic retinal changes is not yet a part of the common thinking about diabetes, but future studies using OCT image segmentation techniques, elaborating histological and functional changes of the macula in diabetic patients may shed light on this very first step possibly leading to the further sequelae of DR.

TABLE 1
Descriptives and total macular thickness (mean ± SD: [min-max])
measurements obtained for each study group (Mann Whitney U test,
‡p < 0.001, and missed significance †p < 0.05).
DM: diabetic eyes with no DR, MDR: eyes with minimal diabetic
retinopathy.

| | Study Groups | |
|---|---|---|
| Descriptives | DM | MDR |
| Number of eyes | 39 | 11 |
| Mean age (SD), yr | 36 ± 10 | 61 ± 20 |
| Female | 19 | 6 |
| Male | 20 | 5 |
| | mean ± SD | |
| Total macular thickness (μm) | [min-max] | |
| Foveal center (ETDRS central region: R1) | 248 ± 23 | 240 ± 18 |
| | [213-326] | [208-278] |

Inner circle (ETDRS pericentral region range: R2-R5)

| | | |
|---|---|---|
| Superior (R2)‡ | 326 ± 11 | 302 ± 11 |
| | [283-364] | [261-329] |
| Temporal (R5)† | 312 ± 10 | 296 ± 6 |
| | [276-352] | [262-322] |
| Inferior (R4)† | 321 ± 9 | 298 ± 7 |
| | [277-356] | [256-325] |
| Nasal (R3)† | 327 ± 11 | 305 ± 10 |
| | [285-368] | [268-343] |

Outer circle (ETDRS peripheral region range: R6-R9)

| | | |
|---|---|---|
| Superior (R6)† | 289 ± 15 | 277 ± 13 |
| | [251-325] | [251-312] |
| Temporal (R9)† | 269 ± 20 | 258 ± 18 |
| | [233-295] | [240-294] |
| Inferior (R8)† | 269 ± 18 | 254 ± 17 |
| | [232-288] | [233-279] |
| Nasal (R7)† | 298 ± 14 | 280 ± 14 |
| | [261-328] | [252-304] |

TABLE 2
Mean intraretinal layer thickness values and total macular
thickness (μm) that showed a statistically significant difference
(Mann-Whitney U test, ‡p < 0.001, and missed significance
†p < 0.05) between diabetic eyes with no DR (DM) and eyes
with minimal diabetic retinopathy (MDR). Values reported are
mean ± SD [range].

| Macular | Intraretinal | | |
|---|---|---|---|
| Region | Layer | DM | MDR |
| Central | OS/RPE | 16 ± 2 | 15 ± 2 |
| | junction | [13-22] | [11-17]^{†} |
| Pericentral | RNFL | 27 ± 2 | 18 ± 5 |
| | | [21-32] | [11-24]^{‡} |
| | GCL + IPL | 92 ± 7 | 80 ± 10 |
| | | [78-104] | [61-94]^{‡} |
| | Macula | 322 ± 16 | 298 ± 20 |
| | | [280-360] | [262-321]^{‡} |
| Peripheral | RNFL | 42 ± 3 | 33 ± 9 |
| | | [35-48] | [20-48]^{‡} |
| | Macula | 282 ± 13 | 267 ± 14 |
| | | [244-309] | [246-295]^{‡} |

### Example 3

### Assessment of Intraretinal Light-Backscatter in Eyes With No or Minimal Diabetic Retinopathy using Optical Coherence Tomography

Purpose: To assess the light-backscatter of intraretinal layers in normal and diabetic eyes with no or minimal diabetic retinopathy using Optical Coherence Tomography (OCT).

Methods: Standard macular mapping by Stratus OCT were performed in 74 healthy eyes (34±16 years) and 26 eyes with diabetes mellitus (DM) with no or minimal diabetic retinopathy (19 eyes no DR [DM; 32±9 years] and 7 eyes with minimal DR [MDR; 63±18]) on biomicroscopy. Automatic layer segmentation was performed using a custom-built algorithm. Mean values of thickness and relative light-backscatter of the RNFL, GCL+IPL, INL, OPL, ONL, IS/OS and RPE in healthy normal, DM and MDR eyes were compared using ANOVA followed by Newman-Keuls post hoc analysis. A p value of <0.05 was considered statistically significant.

Results: Relative light-backscatter was significantly less in DM eyes than in normal (p<0.05 for all the layers except ISOS). However, thickness of the RNFL, ONL and ISOS was not significantly less in DM than in controls. Relative light-backscatter was significantly less in MDR eyes than in normal for only the RNFL, ISOS and RPE (p<0.05). Nevertheless, thickness of the GCL+IPL, OPL and ONL was significantly less in MDR than in healthy eyes. Relative light-backscatter and thickness in the GCL+IPL, INL, OPL and ONL was significantly more in MDR than in DM eyes (p<0.05). No significant differences in light-backscattering between DM and MDR eyes were obtained for ISOS and RPE. There was no difference in RNFL relative light-backscatter and thickness between DM and MDR eyes (45±8% vs. 45±6%, p=0.96; and 41±3 μm vs. 42±4 μm, p=0.38; respectively). On the contrary, RNFL's light-backscatter was significantly less in eyes with DM and MDR than in normal eyes. Moreover, GCL+IPL's thickness in MDR eyes showed a tendency towards thinning as compared with normal and DM eyes. Conversely, ONL and OPL's thickness showed an increasing trend in the MDR group.

Conclusions: Our results suggest that the GCL+IPL complex is more susceptible to initial damage than RNFL when comparing MDR with DM eyes. This may reflect neurodegenerative changes in the diabetic retina. The trend observed for the GCL+IPL's thickness and its relative light-backscattering in MDR eyes might be associated with pathological metabolic changes in the retina. Light-backscattering along with thickness information of the various cellular layers of the retina may provide useful information about the pathological changes in retinal morphology.

### Example 4

### Evaluation of Intraretinal Scattering Measurements in Eyes of Healthy and Type I Diabetic Subjects with No Retinopathy Using Optical Coherence Tomography

Purpose: To assess the scattering measurements of the intraretinal layers in healthy normal and type 1 diabetic eyes using optical coherence tomography (OCT).

Methods: Unprocessed raw scan data were exported from the Stratus OCT machine after performing standard macular mapping in 74 healthy eyes (34±16 yrs, 51 female, 23 male) and 39 eyes with type 1 diabetes mellitus (DM) with no retinopathy (36±10 yrs, 19 female, 20 male) on biomicroscopy. Automatic layer segmentation was performed using a custom-built algorithm (OCTRIMA). Mean values of relative internal reflectivity (RIR) and reflectivity with normalization to the RPE reflectance (NRPE) were used in the comparisons. Mean light-backscattering, contrast measures and scattering coefficients of the RNFL, GCL+IPL, INL, OPL, ONL, IS/OS and OS/RPE junction were calculated. The scattering coefficients were calculated using a finite difference method. Mann-Whitney U test was used to test for differences between the two groups. A modified p value of <0.001 was considered statistically significant. Missed significance (MS, 0.001<p<0.05) was also recorded.

Results: Scattering coefficients were significantly different between DM and healthy eyes for the IS/OS and OS/RPE junction for both types of normalization used (RIR: 9.84±2.05 mm⁻¹ versus 8.49±1.12 mm⁻¹ and 10.44±1.59 mm⁻¹ versus 9.84±1.41 mm⁻¹, ‡p<0.001, respectively and; NRPE: 12.80±1.94 mm⁻¹ versus 14.19±3.02 mm⁻¹ and 14.69±2.34 mm⁻¹ versus 15.18±2.71 mm⁻¹, ‡p<0.001, respectively). The mean light-backscattering and contrast measures between DM and healthy eyes were not significantly different.

Conclusions: These results evidence that the optical properties of the intraretinal layers may provide useful information about the extent of intraretinal layer injury in diabetes. Diabetes inflicts structural damage to the inner retinal segment supported by the thinning of the ganglion cell complex (RNFL+GCL+IPL). However, it appears that diabetes also inflicts additional damage to the outer retinal segment demonstrated by the optical changes of the IS/OS and OS/RPE junction.

### Example 5

### Comparing Intraretinal Scattering Measurements Between Eyes of Healthy Subjects and Type I Diabetic Patients with No or Minimal Diabetic Retinopathy Using Optical Coherence Tomography

Purpose: To assess the scattering measurements of the intraretinal layers in healthy normal and type 1 diabetic eyes using optical coherence tomography (OCT).

Methods: Unprocessed raw scan data were exported from the Stratus OCT machine after performing standard macular mapping in 74 healthy eyes (34±16 yrs, 51 female, 23 male), 39 eyes with type 1 diabetes mellitus (DM) with no retinopathy (36±10 yrs, 19 female, 20 male) and 11 eyes with type 1 diabetes mellitus with minimal (MDR) retinopathy (61±20 yrs, 6 female, 5 male) on biomicroscopy. Automatic layer segmentation was performed using a custom-built algorithm (OCTRIMA). Mean values of relative internal reflectivity were used in the comparisons. Mean light-backscattering and scattering coefficients of the RNFL, GCL+IPL, INL, OPL, ONL, IS/OS and OS/RPE junction were calculated. The scattering coefficients were calculated using a finite difference method. Newman-Keuls test was used to test for differences between the three groups. A modified p value of <0.001 was considered statistically significant. Missed significance (MS, 0.001<p<0.05) was also recorded.

Results: Scattering coefficients were significantly different between MDR and healthy eyes for the IS/OS and OS/RPE junction (7.59±1.71 mm⁻¹ versus 8.49±1.12 mm⁻¹ and 12.14±1.97 mm⁻¹ versus 9.84±1.41 mm⁻¹, ‡p<0.001, respectively). Significant differences were also found between MDR and DM eyes for the IS/OS and OS/RPE junction (7.59±1.71 mm⁻¹ versus 9.24±1.74 mm⁻¹ and 12.14±1.97 mm⁻¹ versus 9.94±1.69 mm⁻¹, ‡p<0.001, respectively).

Conclusions: These results evidence that the optical properties of the intraretinal layers may provide useful information about the extent of intraretinal layer injury in diabetes. Diabetes inflicts structural damage to the inner retinal segment supported by the thinning of the ganglion cell complex (RNFL+GCL+IPL). However, it appears that diabetes also inflicts additional damage to the outer retinal segment demonstrated by the optical changes of the IS/OS and OS/RPE junction.

### Example 6

### Assessment of Macular and Intraretinal Thickness Measurements in Eyes of Healthy Volunteers and Subjects with type 1 Diabetes with No Retinopathy Using Optical Coherence Tomography

Purpose: To assess the thickness measurements of the macula and intraretinal layers in patients with type 1 diabetes mellitus and no retinopathy using optical coherence tomography (OCT); and to compare these findings with those of age-matched healthy volunteers.

Methods: Standard macular mapping by Stratus OCT was performed in 74 healthy eyes (34±16 yrs, 51 female, 23 male) and 39 eyes with type 1 diabetes mellitus (DM) with no retinopathy (36±10 yrs, 19 female, 20 male) on biomicroscopy. Automatic layer segmentation was performed using a custom-built software for OCT retinal image analysis (OCTRIMA). Mean values of thickness of the macula and RNFL, GCL+IPL, INL, OPL, ONL, IS/OS and OS/RPE junction in healthy volunteers and DM eyes were compared using Mann-Whitney U test. Because of the number of statistical comparisons made in the study, a modified p value of <0.001 was considered statistically significant. Missed significance (MS, 0.001<p<0.05) was also recorded.

Results: Stratus OCT-measured thickness of the total retina in the central subfield (R1) of DM eyes was higher than those from healthy volunteers (242±23 versus 232±24, ‡p<0.001). Intraretinal thickness was significantly different between DM and healthy eyes for RNFL, which was thinner in the pericentral regions in DM eyes (R2‡, R3‡, R4† and R5†, ‡p<0.001 and †MS); GCL+IPL complex in R4†, R6†, R7† and R8†, which was also thinner in DM eyes; INL in R2† and R3† (thicker in DM eyes); OPL only in R8† (thicker in DM eyes) and; OS/RPE junction in R1‡, R4‡, R5†, R8† and R9† (thicker in DM eyes). This study also showed no significant differences in macular and intraretinal layer thickness measurements within regions between females and males in the DM group.

Conclusions: In contrast to previous results reported in the literature for subjects with diabetes but minimal or no retinopathy, our results suggest that macular and intraretinal layer thickness measurements in DM subjects are not similar to thickness measurements obtained from age-matched healthy subjects without diabetes. In addition, the differences of the macular and intraretinal layer thickness measurements between men and women in the DM group were not significant.

### Example 7

### Comparison of Retinal Thickness by Fourier-Domain Optical Coherence Tomography and OCTRIMA Segmentation Analysis Derived from Stratus OCT Images

Purpose: To compare thickness measurements and segmentation performance between Fourier-domain optical coherence tomography (FD-OCT) image analysis and time-domain OCT images analyzed with a custom-built OCT retinal image analysis software (OCTRIMA).

Materials and Methods

Patients and Methods: A total of eleven eyes from eleven subjects (9 women and 2 men) were included in this study. Taking into consideration that image quality could be affected by media opacities, elderly subjects were included who had underwent uneventful phacoemulsification surgery with posterior chamber lens (PCL) implantation 6-12 months prior to enrollment. The mean patient age was 70±7 years (range, 65-88 years). The inclusion and exclusion criteria for all participants are listed in Table 3 along with the performed clinical examinations. All subjects were treated in accordance with the tenets of the Declaration of Helsinki. Informed consent was obtained from all participants in this study.

TABLE 3
The inclusion and exclusion criteria for all participants and
the performed clinical examinations.

Inculsion criteria
Best-corrected Snellen visual acuity of 20/20
Preoperative spherical and cylindrical correction within ±3.0 diopters (D)
Exclusion criteria
The presence of any retinal disease including glaucoma
The presence of systemic diseases except for controlled hypertension
Clinical examinations
Best corrected visual acuity (BCVA)
Assessment of intraocular pressure (IOP)
Slit lamp biomicroscopy
Binocular ophthalmoscopy after pupil dilatation

Stratus Stratus OCT and RTVue examinations were performed on each eye by the same examiner and with intervals of approximately 10 minutes. For Stratus OCT measurements Macular Thickness Map (MTM) protocol was performed consisting of six evenly spaced radial lines centered on the fovea, each having a 6 mm transverse length. In order to obtain the best image quality focusing, optimization settings and scans were controlled and were accepted only if signal strength was above 6 (preferably 9-10). Scans with foveal decentration (i.e. with center point thickness SD>10%) were repeated. The mean SD percentage of the center point thickness of the accepted scans was 4.96±2.58%. Stratus OCT raw data were exported and analyzed using OCTRIMA. Algorithm performance using OCTRIMA was subjectively evaluated by a human expert to detect algorithm errors. These errors were manually corrected using the manual correction tool provided by OCTRIMA. For RTVue measurements, MM5 and MM6 protocols were performed. MM5 protocol consists of a dense 5×5-mm grid of linear scans around the macula. MM6 protocol consists of 12 evenly spaced radial lines centered on the fovea with a 6 mm transverse length, similar to the Stratus MTM protocol. According to the AIGS Study recommendations, RTVue scans were included with an SSI≧45, having a range between 48.9 and 82.7.

The Stratus OCT system images the outer retinal layers (RPE-photoreceptor complex) as two hyperreflective bands: 1) the photoreceptor inner/outer segment junction and 2) outer segments interdigitising with the microvilli of the RPE (i.e. the OS/RPE junction). The segmentation software of the Stratus OCT system uses the anterior border of the first or innermost hyperreflective band (i.e. photoreceptor inner segment) as the border of the outer retina for calculating the total retinal thickness. OCTRIMA detects the outer retinal boundary as the anterior border of the second hyperreflective band, which is thought to represent the tip of the cone outer segment in the fovea. Thus, OCTRIMA calculates the total RT as the distance between the vitreoretinal interface (ILM) and the anterior boundary of the second hyperreflective band corresponding to the OS/RPE junction. On the other hand, total RT measurements of RTVue are taken between the ILM and the edge defined by the mean value of the maximum reflectance of the second hyperreflective band (i.e. the OS/RPE junction), which defines the outer retinal border below the protoreceptor inner/outer segment junction (information from the manufacturer, Optovue Inc, Fremont, Calif.).

Retinal thickness measured by Stratus OCT, MM5 and MM6 protocols was compared for each of the nine ETDRS subfields with corresponding OCTRIMA results by analysis of variance (ANOVA) followed by Dunnet post-hoc test with comparisons made to OCTRIMA results. The exact location of regions R1-9 is described in detail in Table 4.

TABLE 4
Retinal thickness values in each ETDRS subfield by each software and the differences
between the measurements (*p < 0.05, †p < 0.01 by Dunnett post-hoc test).

| | | | | | Mean difference of regional thickness | | | |
|---|---|---|---|---|---|---|---|---|
| | | | | | | OCTRIMA | OCTRIMA | RTVue |
| | Mean regional thickness | | | | OCTRIMA | minus | minus | MM6 |
| | | RTVue | RTVue | | minus | RTVue | RTVue | minus |
| | OCTRIMA | MM6 | MM5 | Stratus | Stratus | MM6 | MM5‡ | Stratus |
| R1 | 245 ± 19 | 257 ± 20 | 259 ± 19 | 206 ± 21 | 39 ± 4† | −12 ± 8 | −14 ± 7 | 51 ± 9† |
| (fovea) | | | | | | | | |
| R2 (inner | 314 ± 16 | 312 ± 18 | 318 ± 14 | 269 ± 17 | 45 ± 2† | 2 ± 10 | −4 ± 11 | 44 ± 10† |
| superior) | | | | | | | | |
| R3 (inner | 316 ± 16 | 312 ± 17 | 321 ± 17 | 274 ± 17 | 42 ± 5† | 4 ± 6 | −5 ± 5 | 38 ± 10† |
| nasal) | | | | | | | | |
| R4 (inner | 310 ± 20 | 314 ± 12 | 312 ± 20 | 269 ± 24 | 42 ± 5† | −4 ± 10 | −2 ± 4 | 46 ± 14† |
| inferior) | | | | | | | | |
| R5 (inner | 302 ± 16 | 309 ± 15 | 304 ± 18 | 257 ± 17 | 45 ± 4† | −7 ± 7 | −2 ± 6 | 52 ± 7† |
| temporal) | | | | | | | | |
| R6 (outer | 282 ± 18 | 257 ± 18 | — | 228 ± 21 | 54 ± 8† | 25 ± 9† | — | 29 ± 11† |
| superior) | | | | | | | | |
| R7 (outer | 286 ± 14 | 268 ± 14 | — | 243 ± 16 | 42 ± 6† | 18 ± 7* | — | 25 ± 11† |
| nasal) | | | | | | | | |
| R8 (outer | 260 ± 16 | 265 ± 11 | — | 223 ± 16 | 36 ± 8† | −5 ± 8 | — | 42 ± 12† |
| inferior) | | | | | | | | |
| R9 (outer | 258 ± 17 | 265 ± 14 | — | 213 ± 17 | 45 ± 4† | −7 ± 6 | — | 52 ± 6† |
| temporal) | | | | | | | | |
| Mean | 286 ± 15 | 284 ± 13 | 303 ± 15 | 242 ± 15 | 43 ± 8† | 2 ± 13 | 48 ± 9 | 42 ± 14† |
| thickness | | | | | | | | |
| WMT | 279 ± 15 | 274 ± 13 | — | 235 ± 16 | 44 ± 3† | 5 ± 4 | — | 39 ± 6† |

Data are represented as mean ± SD (Gm).
SD: standard deviation.
‡OCTRIMA and RTVue MM5 average thickness results were not compared because of the different number of regions analyzed.
WMT: weighted mean thickness.

Because of the scan length (5 mm) used in the MM5 protocol only the foveal and pericentral regional (R1-R5) data were used in the analyses. Wilcoxon matched-pairs test was performed to compare the thickness of the GCC measured by RTVue using the MM6 protocol and OCTRIMA with the exclusion of R1 as ganglion cells are not present in the area of the foveal pit. Pearson correlation coefficients were calculated for the above pairwise comparisons. Statistical analyses were performed using Statistica 8.0 Software (Statsoft Inc., Tusla, Okla., USA). The level of significance was set at 5%. Since the sampling is different at each ETDRS region because of different radial spoke patterns used in the scanning protocols of Stratus and RTVue (MM6 protocol), the retinal thickness results measured in the 9 regions were not simply averaged but calculated a weighted mean thickness (WMT). For each eye, WMT was generated, representing an interpolated weighted average. The WMT was calculated using the following equation:

$$WMT = \frac{R 1}{36} + \frac{R 2 + R 3 + R 4 + R 5}{18} + \frac{( R 6 + R 7 + R 8 + R 9 ) \times 3}{16}$$

Bland Altman plots were constructed to assess agreement in WMT measurements.

Results

A high correlation was observed for RT when comparing OCTRIMA with RTVue MM5 and MM6 protocols (Pearson correlation coefficients range: 0.93-0.97 and 0.82-0.94, respectively). Similarly, a high correlation was obtained for the GCC measurements when comparing OCTRIMA with RTVue MM6 protocol (Pearson correlation coefficients range: 0.73-0.88). Analyses of variance (ANOVA) followed by Dunnett post-hoc test have shown no significant differences in regional thickness measurements between OCTRIMA and RTVue except for ETDRS regions R6 and R7 by the MM6 protocol. The mean difference in RT measurements between OCTRIMA, MM6 and MM5 protocols was less than 7 μm in each ETDRS region except for R1, R6 and R7 (see Table 4). OCTRIMA produced significantly thicker measurements for R6 and R7 (25.00±8.84 μm and 17.64±7.46 μm, mean difference ±SD, respectively). In the case of the GCC thickness measurements, the mean difference range was from 6.3 to 12.4 μm (see Table 5). GCC measurements were significantly thicker for the MM6 protocol, except for R6 and R7 where MM6 produced thinner results.

TABLE 5
Mean GCC thickness values measured in each ETDRS subfield by
OCTRIMA and RTVue MM6 protocol.

| | | | OCTRIMA |
|---|---|---|---|
| | | | minus |
| | | RTVue | RTVue |
| | OCTRIMA | MM6 | MM6 |
| R2 | 114 ± 13 | 126 ± 12 | −12 ± 8† |
| R3 | 114 ± 12 | 122 ± 12 | −8 ± 7* |
| R4 | 114 ± 16 | 125 ± 11 | −11 ± 9† |
| R5 | 105 ± 13 | 119 ± 11 | −14 ± 9† |
| R6 | 106 ± 14 | 93 ± 10 | 13 ± 8† |
| R7 | 112 ± 13 | 99 ± 8 | 13 ± 9† |
| R8 | 90 ± 12 | 96 ± 7 | −6 ± 8* |
| R9 | 87 ± 10 | 95 ± 7 | −8 ± 7* |
| WMT | 99 ± 13 | 99 ± 10 | 0 ± 5 |

Data are represented as mean ± SD (μm).
SD: standard deviation.
(*p < 0.05, †p < 0.01 by Wilcoxon test compared to OCTRIMA measurements)
WMT: weighted mean thickness.

Bland-Altman plots for the difference in weighted mean total retinal thickness between OCTRIMA and RTVue MM6 protocol, average total retinal thickness between OCTRIMA and RTVue MM5 protocol, and for the difference in weighted mean GCC thickness between OCTRIMA and RTVue MM6 protocol, show high correlation of the measurements.

Discussion

Optical coherence tomography has become an integral part of ophthalmic clinical practice and plays an increasing role in the diagnosis and management of retinal diseases. Recent studies have shown that currently available FD-OCT devices are giving significantly higher RT measurements than Stratus OCT due to different assumptions considered for the detection of the outer retinal boundary, making it difficult to compare data obtained by different devices. These differences also make it difficult to adequately evaluate the performance of FD-OCT to detect the progression of disease.

The algorithm of Stratus OCT use the anterior border of the innermost hyperreflective band (i.e. photoreceptor IS) as the border of the outer retina for calculating the total retinal thickness. In contrast, FD-OCT devices image the outer retinal layers (RPEphotoreceptor complex) as three hyperreflective bands: 1) IS/OS junctional complex, 2) outer segments interdigitising with the microvilli of the RPE (i.e. the OS/RPE junction) and 3) RPE cell bodies, although reflections from the choriocapillaries might also be included. This gives rise for a high variability in retinal thickness measurements. In previous studies, the largest difference in total RT measurements in normal subjects compared to Stratus OCT results was found for Spectralis OCT (Heidelberg Engineering, Heidelberg, Germany), as Spectralis calculates the RT between the ILM and the outer border of the RPE layer. The Cirrus OCT (Carl Zeiss Meditec, Inc., Dublin, Calif.) system uses the inner border of the second hyperreflective band as the outer border of the retina. Accordingly, recent studies have found that Cirrus measured the retina 41.9-65 μm thicker than Stratus. SOCT Copernicus (Optopol Technology SA, Zawiercie, Poland), Spectral OCT/SLO (OTI, now a division of OPKO, Miami, Fla.) and RTVue show similar differences (30.9-41.9 μm) in the RT compared to Stratus OCT measurements which can indicate that the outer retinal border detection of these devices is similar.

Total retinal thickness on RTVue images is calculated between the ILM and the edge defined by the mean value of the maximum reflectance of the OS/RPE junction in order to avoid detection errors at the junction's outer border. In this study, RTVue's MM6 protocol was found to measure the retina 42±14 μm thicker compared to Stratus OCT results. These results are comparable to the results obtained by Wolf-Schnurrbusch et al. (Invest Ophthalmol Vis Sci 2009). In contrast, Menke et al. (Ophthalmologica 2009; 223:352-356) and Huang et al. (Retina 2009; 29:980-987) reported a difference of only 14 μm and 8 μm respectively, after comparing Stratus and RTVue measurements. The reason for this difference is unclear.

The software of Topcon 3D OCT-1000 (Topcon Inc., Tokyo, Japan) allows users to set their thickness measurements from the ILM to RPE layer, as defined in the traditional histopathology, or measure the thickness from the ILM to the inner border of the IS/OS photoreceptor junction, for consistency with the legacy time domain system. However, no information is provided about which boundary of the RPE layer (i.e. inner or outer) is detected.

On the contrary, OCTRIMA measures RT as the distance between the ILM and the inner boundary of the OS/RPE junction defined as “true retinal thickness”. The data herein show a 43±8 μm mean difference between RT values measured by OCTRIMA derived from Stratus OCT images which is very similar to the mean difference between RTVue and Stratus OCT. Bland-Altman plots showed that WMT measured by OCTRIMA is on average 5 μm higher than that measured by RTVue using the MM6 protocol and is on average 5 μm lower than that measured by RTVue using the MM5 protocol which both are below the axial resolution of the devices. The explanation for the comparable differences might be that the location of the mean value of the maximum reflectance of the second hyperreflective band calculated by RTVue and used to define the outer border of the retina is closely located to the actual inner border of the second hyperreflective band (i.e. the OS/RPE junction). Thus, the small differences between the two measurement methods of RTVue and OCTRIMA are hardly distinguishable. A high correspondence for the RT measurements was obtained, herein, when these measurements were compared between OCTRIMA and RTVue using MM5 and MM6 protocols. The observed good correlation emphasizes the capability of comparable retinal measurements by Fourier-domain OCT and Stratus OCT-derived segmentation using the OCTRIMA software.

It should be noted that RT measurements obtained in R1 by the MM5 and MM6 protocols of RTVue were approximately 12-14 μm higher than measurements obtained with OCTRIMA, however not reaching a statistical difference. The reason for this difference is not clear as Stratus OCT scans were carefully centered, the mean SD % of the center point thickness of the accepted scans was 4.96±2.58%, therefore the difference is unlikely to have been caused by decentration artifact. Moreover, the OCTRIMA algorithm used in this study is optimized for Stratus OCT images and segmentation errors were manually corrected by an experienced operator. Theoretically, FD-OCT should produce fewer segmentation failures because of higher resolution. Although RTVue allows manual correction of the automated segmentation, the manual adjustment was not used to change the segmentation results, while differences in axial resolution and calibration might also contribute to thickness measurement differences.

Besides, mean retinal thickness values were generated with different scan protocols with the sampling density being higher in the outer ETDRS areas by the RTVue MM6 protocol (12 radial scans) compared to Stratus OCT (6 radial scans) which might also contribute to the difference between the thickness measurements in R1. In the outer superior and outer nasal subfields (i.e. R6 and R7), the RTVue results obtained with the MM6 protocol produced significantly thinner results than OCTRIMA, by approximately 18-25 μm. A possible explanation for the above differences could be related to incorrect boundary detection, however OCTRIMA segmentation errors were manually corrected by an experienced operator. Particularly, minor errors in the segmentation of the outer border of the retina were observed at the periphery in some RTVue scans. Taking into consideration the known anatomical properties of the regions in the peripheral ring (the nasal macula being thicker than the temporal) it was supposed that thickness values measured by the RTVue device should have been higher in subfields 6 and 7. Thus, it was inferred that RTVue's thinner measurements in R6 and R7 were possibly due to the low quality observed in some OCT scans, which could be a direct result of image acquisition pitfalls leading to segmentation errors.

In this study, RTVue peripheral regions R6-R9 (using the MM6 protocol) had all comparable thickness values as opposed to Stratus OCT data being distributed as described above. Similar results were found by Huang et al. when comparing regional thickness measurements between Stratus OCT and RTVue OCT. They found that RTVue produced significantly higher RT measurements in each ETDRS region except for the outer inferior region (R8) where the difference was not significant and the outer nasal region (R7) where Stratus OCT produced thicker measurements than RTVue, however, no explanation was found for these differences.

It is also of great interest that the software of the RTVue device enables the segmentation of the ganglion cell complex (GCC) in the macula by detecting the IPL outer boundary, which might facilitate a more rigorous glaucoma analysis. (Tan O, et al. IOVS 2007; 48:ARVO E Abstract 512). Certainly, OCTRIMA is also able to extract the GCC, therefore the potentialities of the two algorithms were compared. A good correspondence between OCTRIMA and RTVue MM6 protocol was found, however, thickness values measured by the RTVue device were 6-10 μm higher in all but two regions. Interestingly, GCC in subfields R6 and R7 was approximately 12 μm thinner by the RTVue using the MM6 protocol than by OCTRIMA measurements, similarly to total retinal thickness values. This difference was less than that observed for the total RT, therefore it was assumed it was not an isolated GCC or outer retinal boundary detection error. Although the above difference in GCC measurements is relatively small, it could be of great importance in clinical settings as for example in glaucoma diagnostics. The small average difference of 1 μm shown by the Bland-Altman plot might be due to the results in R6, R7 influencing the calculation of the mean.

In summary, it was found that measurements with the Stratus OCT showed the lowest RT values, whereas measurements with the RTVue OCT and Stratus OCT-derived images assessed by OCTRIMA yielded the highest ones. These discrepancies were based on differences in retinal segmentation algorithms. In addition, a high correspondence of RT measurements between Fourier-Domain OCT and Stratus OCT-derived images assessed by OCTRIMA was demonstrated. Despite the worse resolution of TD-OCT we could achieve a high correspondence of retinal layer segmentation with FD-OCT in elderly subjects who are supposed to have bad fixation cooperation. Weighted mean total retinal thickness data were shown to have high correlation, while regional differences might still exist. The measurements of the GCC should also be compared with care as there is a marked difference between OCTRIMA and RTVue using the MM6 protocol. These differences were most probably based on differences in retinal segmentation algorithms, sampling, calibration and axial resolution.

An agreement between ophthalmologists and developers is needed in order to standardize OCT RT measurements, however, the use of custom-built segmentation software along with open-source image files enabling their easy access could also facilitate the transformation of data obtained by different devices. In view of the higher price of FD-OCT systems and the widespread use of TD-OCT worldwide, we believe our OCTRIMA software can be of substantial value in future studies of macular pathophysiology and it might also perform well for FD-OCT images in the future.

### Example 8

### Reliability and Reproducibility of Macular Segmentation Using a Custom-built Optical Coherence Tomography Retinal Image Analysis Software

Methods (Analysis Per Scan: Uninterpolated Data)

Subjects: Ten undilated eyes of five healthy subjects ranging in age from 25 to 34 yr (mean age 29 yr), were involved in this study. Inclusion criteria included best-corrected visual acuity of 20/25 or better, no history of any current ocular or systematic disease, and a normal appearing macula on contact lens biomicroscopy. All subjects underwent visual acuity testing with refraction and a complete slit-lamp examination. All subjects were treated in accordance with the tenets of the Declaration of Helsinki.

OCT Measurements: For imaging purposes the commercially available Stratus OCT unit (software version 4.0; Carl Zeiss Meditec, Inc., Dublin, Calif.) was used. Subjects underwent three OCT scanning sessions during the first visit on day (D1) by two experienced examiners (E1, E2) with intervals of approximately 5 min between scans (sessions 1 and 2, corresponding to S1 and S2, respectively). Thus, two scans (E1 D1S1, E1D1S2) were performed by the same examiner (E1) to determine intraobserver repeatability (i.e., E1D1S1 versus E1D1S2). A third scan was performed by a second examiner (E2) and the results were compared with those of the first scan (SD to determine interobserver reproducibility (i.e., E1D1S1 versus E2D1S1). To assess inter-visit reproducibility (i.e., E1D1S1 versus E1D2S1), an additional scan session was performed by one of the examiners (E1) during a second visit (D2) the next day. Between the examiners, the OCT instrument alignment and controls were randomly changed, so all alignment and focusing had to be restarted. Only scans with a signal strength of 6 or more were accepted.

The Radial Lines protocol was used for the OCT studies. This protocol acquires six retinal B-scans each of scan length 6 mm, each scan oriented 30 deg apart from each other, and centered at the fovea. Each B-scan consists of 512 aligned A-scans. Each A-scan consists of 1024 pixels with a total scan depth of 2 mm in tissue. Thus, each B-scan acquired in this protocol consists of a total 1024×512 pixels. If the subject moved or blinked during the scan, the image was repeated. In addition, the quality of B-scans was evaluated with OCTRIMA. Generally, the standard deviation of the foveal center point thickness is used as a measure of the scan variance. A high standard deviation (>10% of center point thickness) indicates high variability, usually due to patient movement or boundary line error, and therefore incorrect center point thickness. Good quality images have a standard deviation <10% of center point, good clarity of the layers, and are also well centered. Substantially decentered scans could have a low standard deviation. Therefore, a scan quality factor (SQF) based on the standard deviation calculation (in percent) of the foveal center point (FCP) for the six radial line scans included in the OCTRIMA software was used to control the variability of measurements associated to image acquisition pitfalls. A good scan has an SQF=1, indicating that the percentage standard deviation of the foveal center point is <10. Data for each measurement were exported to disk using the export feature available in the Stratus OCT version 4.0 analysis software.

Computer-Aided OCT Image Analysis Software: OCTRIMA is a powerful computer-aided system designed to facilitate viewing and automatic/semiautomatic OCT retinal image analysis. The application essentially provides dual functionality in a single software package by combining image enhancement and speckle denoising of Stratus OCT images along with intraretinal segmentation and error correction using direct visual evaluation of the detected boundaries. Moreover, the software has the capability to provide quantitative analysis based on measured values of corrected thickness, volume, and reflectance of the various cellular layers of the retina. A total of seven intraretinal layers can be extracted using OCTRIMA, namely, the retinal nerve fiber layer (RNFL), the ganglion cell layer along with the inner plexiform layer (GCL+IPL), the inner nuclear layer (INL), the outer plexiform layer (OPL), the outer nuclear layer (ONL), the photoreceptor inner/outer segment (IS/OS) junction, and the outer segment/retinal pigment epithelium (OS/RPE) junction.

Quantitative Analysis: As a result of repeatedly scanning a total of 10 healthy eyes during four sessions on two consecutive days by two OCT examiners, a total of 240 OCT B-scans were collected and analyzed by an experienced grader. Specifically, the grader segmented all the B-scans from all sessions (E1D1S1, E1D1S2, E2D1S1, and E1D2S1) using OCTRIMA for testing the intraobserver, interobserver, and intervisit variability of repeated measurements performed by the same examiner, by different examiners, and at different visits.

After each B-scan was denoised, the inner and outer borders of the retinal structure were identified between the internal limiting membrane (ILM) and the inner boundary of the OS/RPE junction; and a total of seven intraretinal layers were extracted using OCTRIMA. Note that visualizing and quantifying microstructural changes within the photoreceptor and RPE, layers is difficult using Stratus OCT images due to weakened signal energy after penetrating the neuroretina, RPE, and choriocapillaries. Thus, the three outermost hyperreflective layers clearly observed with Fourier domain OCT systems are not certainly visible in Stratus OCT images. Only two hyperreflective layers and one hyporeflective band are observed with the Stratus OCT device. These layers have been identified as the IS/OS junctional complex, which is the first hyperreflective layer, the hyporeflective band below this junction, which is clearly wider in the fovea and attributed to the photoreceptor OSs, and the second hyperreflective layer corresponding to the outer segments interdigitizing with the microvilli of the RPE (i.e., the OS/RPE junction). In time domain OCT images, the RPE and photoreceptor OSs are too close to be resolved and often appeared as a single hyperreflective band. Thus, the second and third hyperreflective layers have been conventionally assigned to the RPE in previous studies using Stratus OCT images. However, the third hyperreflective layer only visible in SDOCT images and identified as the RPE, is probably due to a signal from the RPE cell bodies, although reflections from choriocapillaries might also be included. Accordingly, OCTRIMA measurements of the total retinal thickness were made from the innermost point of the retina (ILM) to the inner border of the second hyperreflective band, which has been attributed to the OS/RPE junction in agreement with histological studies. Note that this thickness differs from the thickness measured with Stratus OCT, which calculates the distance between the inner border (ILM) of the retina and the inner border of the highly reflective photoreceptor IS/OS junction (i.e., the first hyperreflective band). Therefore, in contrast to OCTRIMA, thickness calculated with the Stratus OCT algorithm does not take into account the thickness of the junctions of the inner/outer photoreceptor segment and the outer photoreceptor segments (i.e., the hyporeflective band) in the fovea.

All scans in the study had a signal strength of 9 or 10 and were perfectly centered (SQF=1). Algorithm performance was visually evaluated by the experienced grader to detect algorithm errors. Criteria for algorithm error included evident disruption of the detected boundary (e.g., small peaks, linear and curve offsets), and/or detected boundary jumping to and from different anatomical structures. The average number of manual corrections needed per scan was three. Since the thickness of the inner and outer photoreceptor segments has been found to be relatively constant, which is consistent with an anatomically uniform thickness, the outer border of the photoreceptor segment junction (IS/OS) can be extracted manually using the semiautomated approach in OCTRIMA. Thus, the outer border of the IS/OS is located 10 pixels from the outer border of the ONL, which gives a constant thickness of 20 μm. Accordingly, the thickness measurements for the IS/OS were not included in this study. Thus, the thickness measurements of the total retina and six intraretinal layers (RNFL, GCL+IPL, INL, OPL, ONL, and OS/RPE junction) were actually used in the analysis. Note that the repeatability and reproducibility analysis was performed for the uninterpolated measurements at every A-scan location for all six B-scans (i.e., uninterpolated raw data).

Statistical Methods: The coefficients of repeatability and reproducibility were calculated along with the intraclass correlation coefficients (ICCs) with the methods outlined by Bland and Altman for each of the uninterpolated averaged thickness measurements obtained for the total retina and intraretinal layer (British Standards Institution, “Accuracy (trueness and precision) of measurement methods and results: basic methods for the determination of repeatability and reproducibility of a standard measurement method,” BS ISO 5725 part 2, British Standards Institution, London (1994); J. M. Bland and D. G. Altman, “Statistical methods for assessing agreement between two methods of clinical measurement,” Lancet 1, 307-310 (1986)). The coefficients of repeatability and reproducibility were computed from the standard deviations (SDs) of the differences between measurements made at each session. The Wilcoxon signed rank test (5% significance level) was performed to determine any statistically significant difference between the measurements obtained by different examiners or during different visits. The ICC was calculated on the basis of a two-way mixed model for analysis of variance (ANOVA) as proposed by Bartko and Carpenter (J. Nerv. Ment. Dis. 163, 307-317 (1976)). The statistical analysis was performed using the software package SPSS version 16 (SPSS Inc., Chicago, Ill.).

Results:

Retinal thickness measurements at every A-scan location for all six B-scans of all 10 eyes was performed for the total retina and six intraretinal layers. As a result, the average thickness per layer was calculated using the OCTRIMA software for each macular scan group of all 10 eyes for each session. Coefficients of repeatability and reproducibility for the total retina and six intraretinal layers are given in Table 6.

TABLE 6
Thickness measurements (mean ± SDs), coefficients of
repeatability/reproducibility (CRs), ICCs, and Wilcoxon test
results obtained for the total retina and six intraretinal layers.

| | | | | |
|---|---|---|---|---|
| Mean ± SD (μm) | CR (μm) | CR (%) | ICC | p Value |

Measures of Repeatability (Intraobserver Test)

| | | | | | |
|---|---|---|---|---|---|
| RNFL | 40.66 ± 1.74 | 1.88 | 4.62 | 0.86 | 0.65 |
| GCL + IPL | 73.45 ± 7.73 | 3.41 | 4.64 | 0.98 | 0.39 |
| INL | 34.13 ± 1.12 | 2.10 | 6.15 | 0.63 | 0.17 |
| OPL | 32.53 ± 0.63 | 1.66 | 5.11 | 0.37 | 0.07 |
| ONL | 88.30 ± 4.92 | 2.91 | 3.29 | 0.96 | 0.24 |
| OS/RPE | 12.72 ± 1.38 | 1.25 | 9.80 | 0.90 | 0.39 |
| Total Retina | 282.23 ± 13.36 | 8.62 | 3.06 | 0.94 | 0.24 |

Measures of Reproducibility (Interobserver Test)

| | | | | | |
|---|---|---|---|---|---|
| RNFL | 40.87 ± 2.11 | 1.88 | 4.61 | 0.90 | 0.33 |
| GCL + IPL | 72.89 ± 8.30 | 4.54 | 6.23 | 0.96 | 0.09 |
| INL | 34.29 ± 1.07 | 1.81 | 5.29 | 0.69 | 0.05 |
| OPL | 32.44 ± 0.78 | 1.68 | 5.18 | 0.54 | 0.17 |
| ONL | 88.11 ± 5.16 | 5.33 | 6.05 | 0.87 | 0.45 |
| OS/RPE | 12.85 ± 1.30 | 3.94 | 30.69 | 0.25 | 0.96 |
| Total Retina | 281.33 ± 15.66 | 12.83 | 4.57 | 0.63 | 0.72 |

Measures of Reproducibility (Intervisit Test)

| | | | | | |
|---|---|---|---|---|---|
| RNFL | 40.91 ± 1.84 | 2.54 | 6.20 | 0.78 | 0.45 |
| GCL + IPL | 73.67 ± 7.66 | 2.22 | 3.02 | 0.99 | 0.96 |
| INL | 34.03 ± 1.12 | 1.61 | 4.72 | 0.76 | 0.39 |
| OPL | 32.45 ± 0.67 | 1.49 | 4.59 | 0.51 | 0.09 |
| ONL | 88.35 ± 4.81 | 3.21 | 3.63 | 0.94 | 0.58 |
| OS/RPE | 12.76 ± 1.46 | 2.00 | 15.68 | 0.78 | 0.58 |
| Total Retina | 281.94 ± 13.58 | 6.69 | 2.38 | 0.97 | 0.29 |

The means and SDs of the differences between measurements obtained under different conditions, ICCs, and Wilcoxon test results are also shown in Table 6. Repeatability coefficients to test intraobserver variability were less than 4% for the total retina and less than 7% for all intraretinal layers except the OS/RPE junction (˜10%). Reproducibility coefficients to test interobserver variability were less than 5% for the total retina and less than 7% for all intraretinal layers except the OS/RPE junction (˜31%); and for intervisit variability it was less than 3% for the total retina and less than 7% for all intraretinal layers except the OS/RPE junction (˜16%) (see Table 6). The ICCs obtained for the intraobserver and intervisit variability tests were greater than 0.75 for the total retina and all intraretinal layers, except INL (intraobserver and interobserver test) and OPL (intraobserver, interobserver, and intervisit test). The lowest ICC values for the total retina were obtained for the interobserver variability test (see Table 6). In addition, the Wilcoxon paired measurements test (5% significance level) showed that there were no statistically significant differences between measurements obtained by different examiners or during different visits.

Discussion: Although a layer-editing tool to manually adjust the retinal layer boundaries for macula and RNFL was recently incorporated in the current Stratus OCT software, its quantitative analysis does not provide thickness measurements of the various intraretinal layers. This limitation in the Stratus OCT system has stimulated interest in developing segmentation algorithms to better detect the local changes in the retinal structure to improve retinal disease detection and its progression.

In this study, the reliability and reproducibility of macular segmentation mapping is reported using the OCTRIMA software, which overcomes the limitation of the Stratus OCT software and provides additional quantitative information that can be extracted from OCT data. The uninterpolated average thickness measurements recorded for all 10 healthy eyes showed that the coefficient of repeatability was less than 4% for the total retina and less than 7% for intraretinal layers except the OS/RPE junction (˜10%). These values indicate high repeatability of the results of measurements generated by the OCTRIMA software (see Table 6). The high variability in the thickness measurements of the OS/RPE junction is due to the fact that the outer boundary of this layer is not clearly visualized in Stratus OCT images because of the low contrast between the OS/RPE junction (outer border) and the RPE inner boundary, which can be attributed to the limitation of the Stratus OCT system to penetrate deeper structures in the retina. Moreover, the interobserver coefficients of reproducibility calculated for the total retina and intraretinal layers (except for the RNFL) were higher than corresponding values for intervisit reproducibility, which may possibly be explained by the fact that subject fatigue and normal drying of the eye during repeated sessions the same day induced more noise into the overall measurements.

In addition, the scans were not aligned between visits because the Stratus OCT did not provide this feature. Thus, this limitation affected the intervisit variability results. Furthermore, there was less than 5% interobserver variability for the total retinal thickness measurements (see Table 6). This is a reassuring finding for an analysis software tool applied to data obtained with a diagnostic instrument, because comparisons of measurements taken for the same subject over a period of time may be compared even when measurements are obtained by different experienced examiners. In summary, intraobserver, interobserver, and intervisit variability combined accounted for less than 5% of total variability for the total retinal thickness measurements and less than 7% for the intraretinal layers except the OS/RPE junction.

Conclusions:

It was ascertained that retinal thickness measurements for the total retina and intraretinal layers (except the OS/RPE junction) performed using OCTRIMA are very repeatable and reproducible. High measurement repeatability and reproducibility is a prerequisite for quantitative application of OCTRIMA in research and clinical work. These findings are particularly useful because they indicate that any retinal thickness change of greater than 5% (or layer average thickness change greater than 7%) in the macular area in healthy undilated subjects are likely to be caused by changes in retinal thickness rather than by inconsistencies in either the OCTRIMA software or in measurements given by the OCT system.

Although the OCTRIMA quantitative analysis of Stratus OCT images described in this paper is potentially useful, new OCT technologies, such as spectral domain, ultrahigh resolution, and adaptive-optics-based OCT technology, are likely to partially afford better solutions to the limitation of existing OCT software by providing images with higher resolution along with a dense map of the retina with precise registration and localization. However, automatic segmentation algorithms for OCT data have a tendency to give erroneous segmentation results especially in pathological cases, which is actually a result of the algorithm performance independently of how well the OCT image could be reproduced with a high level of detail. Consequently, to improve the practicability of OCT technology in ophthalmology, effective data processing requires robust and accurate segmentation algorithms integrated into intelligent software solutions. In addition, computer-aided detection and diagnosis based on automatic/semiautomatic robust algorithms will be essential in clinical studies where large data sets will be impractical for manual grading approaches.

Even though the results presented are based on Stratus OCT images, the main purpose was to establish the feasibility of the quantitative methodology for OCT image analysis independent of the technology used. There is no doubt that if this methodology works well for Stratus OCT images, then it should perform better for SDOCT images which have better resolution. As a matter of fact, OCTRIMA is currently able to analyze B-scans from SDOCT systems. However, a more practical interface to handle the large quantities of measured raw data generated by these systems along with the associated substantial processing is required, and it is currently under development.

In addition, recent studies have shown that retinal thickness measurements between SDOCT devices are significantly different due to different assumptions considered for the detection of the outer retinal boundary, which makes it difficult to compare data obtained by different devices. These differences also make it difficult to adequately evaluate the performance of SDOCT to detect the progression of disease. OCTRIMA could facilitate a well-defined and standardized quantitative analysis for the assessment of retinal diseases and its progression using SDOCT images. Thus, quantitative evaluation of OCT images with OCTRIMA may improve the quality of data and analysis currently being obtained with Stratus OCT and SDOCT devices. From a clinical point of view, it would be possible to understand the mechanism and time-dependence of macular dystrophies and degenerations, and neurodegenerative diseases by understanding the cellular changes of the macula by using the OCTRIMA software. OCTRIMA can be used as an in vivo tool for quantification of the early structural changes in retinal diseases.

Direct comparison of this study with previous reproducibility and repeatability studies is difficult because the age group of healthy subjects along with image segmentation and experimental and statistical methods vary between studies. As with previous findings, change of examiner did not significantly affect the reproducibility of the measurements in healthy eyes. Future studies will examine the repeatability and reproducibility of macular segmentation mapping with OCTRIMA for each of the nine Early Treatment of Diabetic Retinopathy Study (ETDRS)-like regions in healthy subjects and patients with early diabetic retinopathy and other retinal diseases.

### Example 9

### Assessing the Regional Reliability and Reproducibility of Intraretinal Layer Segmentation with a Custom-built OCT Retinal Image Analysis Software

This study investigated the reliability and reproducibility of the OCTRIMA software for each of the 9 ETDRS-like regions (i.e. interpolated measurements) using Stratus OCT data from normal healthy eyes.

Methods

Subjects: A total of ten undilated eyes of five healthy subjects were involved in this study, ranging in age from 25 to 34 yr (mean age 29 yr). All subjects underwent visual acuity testing with refraction and a complete slit-lamp examination. Inclusion criteria included best-corrected visual acuity of 20/25 or better, no history of any current ocular or systematic disease, and a normal appearing macula on contact lens biomicroscopy. All subjects were treated in accordance with the tenets of the Declaration of Helsinki.

OCT measurements: The Stratus OCT unit (software version 4.0; Carl Zeiss Meditec, Inc., Dublin, Calif.) was used to obtain the OCT images from all the subjects. Only scans with a signal strength of six or more were accepted. Subjects underwent three OCT scanning sessions during the first visit on day (D1) by two experienced examiners (E1, E2) with intervals of approximately five minutes between scans (Session 1 and 2, corresponding to S1 and S2, respectively). Thus, two scans (E1D1S1, E1D1S2) were performed by the same examiner (E1) to determine intraobserver repeatability (i.e., E1D1S1 versus E1D1S2). A third scan was performed by a second examiner (E2) and the results were compared with those of the first scan (S1) to determine interobserver reproducibility (i.e. E1D1S1 versus E2D1S1). To assess intervisit reproducibility (i.e., E1D1S1 versus E1D2S1), an additional scan session was performed by one of the examiners (E1) during a second visit (D2) the next day. Between the examiners, the OCT instrument alignment and controls were randomly changed, so all alignment and focusing had to be restarted.

The radial lines protocol was used for the OCT studies. This protocol acquires six retinal B-scans each of scan length 6 mm, each scan oriented 30 deg apart from each other, and centered at the fovea. Each B-scan consists of 512 aligned A-scans. Each A-scan consists of 1024 pixels with a total scan depth of 2 mm in tissue. Thus each B-scan acquired in this protocol consists of a total 1024×512 pixels. If the subject moved or blinked during the scan, the image was repeated. In addition, the quality of B-scans was evaluated with OCTRIMA. Specifically, a scan quality factor (SQF) based on the standard deviation calculation (in %) of the foveal center point (FCP) for the six radial line scans included in the OCTRIMA software was used to control the variability of measurements associated to image acquisition pitfalls. A good scan has a SQF=1, indicating that the percentage standard deviation of the foveal center point is ≦10. Data for each measurement were exported to disk using the export feature available in the Stratus OCT version 4.0 analysis software.

Computer-Aided OCT image analysis software: OCTRIMA is a powerful computer-aided system designed to facilitate viewing and automatic/semiautomatic OCT retinal image analysis. The application essentially provides dual functionality in a single software package by combining image enhancement and speckle denoising of Stratus OCT images along with intraretinal segmentation and error correction using direct visual evaluation of the detected boundaries. Moreover, the software has the capability to provide quantitative analysis based on measured values of corrected thickness, volume, and reflectance of the various cellular layers of the retina. A total of seven intraretinal layers can be extracted using OCTRIMA, namely, the retinal nerve fiber layer (RNFL), the ganglion cell layer along with the inner plexiform layer (GCL+IPL), the inner nuclear layer (INL), the outer plexiform layer (OPL), the outer nuclear layer (ONL), the photoreceptor inner/outer segment (IS/OS) junction; and the outer segment/retinal pigment epithelium (OS/RPE) junction.

Quantitative analysis: A total of 240 OCT B-scans were collected and analyzed by an experienced grader after two OCT examiners repeatedly scanned a total of ten healthy eyes during four sessions in two consecutive days. Specifically, the grader segmented all the B-scans from all sessions (E1D1S1, E1D1S2, E2D1S1, and E1D2S1) using OCTRIMA for testing the intraobserver, interobserver, and intervisit variability of repeated measurements performed by the same examiner, by different examiners, and at different visits.

Once each B-scan was denoised, the inner and outer borders of the retinal structure were identified between the ILM and the inner boundary of the OS/RPE junction. A total of seven intraretinal layers were extracted using OCTRIMA. It is well known that it is difficult to visualize and quantify changes within the photoreceptor and RPE layers using Stratus OCT images because of these layers are too close to be resolved and often appear as a single hyperreflective band in TD-OCT images. Accordingly, OCTRIMA measurements of the total retinal thickness were made from the innermost point of the retina known as the inner limiting membrane (ILM) to the inner border of the second hyperreflective band, which has been attributed to the OS/RPE junction in agreement with histological studies. It was noted that Stratus OCT calculates the total retinal thickness between the inner border (ILM) of the retina and the inner border of the highly reflective photoreceptor IS/OS junction (i.e., the first hyperreflective band). Therefore, in contrast to OCTRIMA, thickness calculated with the Stratus OCT algorithm does not take into account the thickness of the junctions of the inner/outer photoreceptor segment and the outer photoreceptor segment (i.e. the hyporeflective band) in the fovea.

All scans in the study had a signal strength of 9 or 10. Algorithm performance was visually evaluated by experienced graders to detect algorithm errors. Criteria for algorithm error included evident disruption of the detected boundary (e.g. small peaks, linear and curve offsets), and/or detected boundary jumping to and from different anatomical structures (i.e. false segmentation). The average number of manual corrections needed per scan was three. Since the thickness of the inner and outer photoreceptor segments has been found to be relatively constant, which is consistent with an anatomically uniform thickness, the outer border of the photoreceptor segment junction (IS/OS) can be extracted manually using the semi-automated approach in OCTRIMA. Thus, the outer border of the IS/OS is located 10 pixels from the outer border of the ONL, which give a constant thickness of 20 μm. Accordingly, the thickness measurements for the IS/OS were not included in this study. As a result, the thickness measurements of the total retina and six intraretinal layers (RNFL, GCL+IPL, INL, OPL, ONL, and OS/RPE junction) were actually used in the analysis.

In addition, OCTRIMA is capable of generating topographic maps for macular thickness similar to the standards set by the Early Treatment Diabetic Retinopathy Study (ETDRS). An OCTRIMA macular map is divided into nine zones that correspond to the ETDRS regions: fovea within a diameter of 1 mm centered on the foveola; pericentral ring, the circular band from the central 1 mm to 3 mm, divided into four quadrants i.e. superior, inferior, temporal, and nasal; and peripheral ring from 3 mm up to 6 mm, divided into the same quadrants. As in the current Stratus OCT software, it was noted that the OCTRIMA software maps the macula using 6 radial lines centered on fixation. This pattern has higher sampling density in the fovea, which does not contain the following intraretinal layers: RNFL, GCL, IPL, INL, and OPL. Since these particular layers are not present in the foveal region, OCTRIMA uses a predefined control in a 1.5 diameter zone in the fovea. Specifically, the control forces the inner and outer side of the GCL+IPL complex, and the outer side of the INL, and OPL to be coincident in this region.

The repeatability and reproducibility analysis was performed for each of the 9 ETDRS-like regions (R1-R9). The macular regions were defined as R1 fovea, R2 superior inner, R3 nasal inner, R4 inferior inner, R5 temporal inner, R6 superior outer, R7 nasal outer, R8 inferior outer, R9 temporal outer. The central R1 region has a diameter of 1 mm, regions R2 to R5 are zones of a circle 3 mm in diameter, and regions R6 to R9 are zones of a circle 6 mm in diameter.

Statistical methods: The coefficients of repeatability and reproducibility were calculated along with the intraclass correlation coefficients (ICCs) with the methods outlined by Bland and Altman for each of the averaged thickness measurements obtained for the total retina and intraretinal layers. The coefficients of repeatability and reproducibility were computed from the standard deviations (SDs) of the differences between measurements made at each session. The Wilcoxon signed rank test (5% significance level) was performed to determine any statistically significant difference between the measurements obtained by different examiners or during different visits. The ICC was calculated on the basis of a two-way mixed model for analysis of variance (ANOVA) as proposed by Bartko and Carpenter (J Nery Ment Dis 1976; 163:307-317). The statistical analysis was performed using the software package SPSS version 16 (SPSS Inc, Chicago, Ill.).

Results

The average thickness calculated using the OCTRIMA software for each of the 9 ETDRS regions of all 10 eyes for each scanning session is given in Table 7. The same grader (G1) analyzed the scans from all sessions. The repeatability and reproducibility results are shown in Table 8. The coefficient of repeatability (intraobserver variability) was less than 5% for the total retina, less than 17% for the RNFL in the pericentral (R2-R5) and peripheral (R6-R9) regions respectively (except in temporal regions R5 and R9); less than 9% and 21% for the GCL+IPL complex in the pericentral and peripheral regions respectively; less than 14% and 23% for the INL in the pericentral and peripheral regions respectively; and less than 12% and 22% for the OPL in the pericentral and peripheral regions respectively. It was also less than 7% and 6% for the ONL in the pericentral and peripheral regions respectively (see Table 8, upper section). The coefficient of reproducibility (interobserver variability) was less than 7% for the total retina; less than 20% for the RNFL in the pericentral and peripheral regions (except in the temporal regions R5 and R9); less than 11% for the GCL+IPL complex in the pericentral regions and at worst it was 22% (i.e., in R8). It was also less than 15% in the pericentral regions for the INL, OPL, and ONL; less than 15% in the peripheral regions for the ONL; and at worst it was 23-30% (i.e., in R7) for the INL and OPL (see Table 8, middle section). The coefficient of reproducibility (intervisit variability) was less than 5% for the total retina; less than 13% for the RNFL except in R4-R5 and R8-R9, less than 9% for the GCL+IPL complex in the pericentral regions and at worst it was 16% (i.e., in R8). It was also less than 15% (18%) and 17% (20%) for the INL (OPL) in the pericentral and peripheral regions respectively; and less than 8% for the ONL (see Table 8, lower section). The coefficients of repeatability and reproducibility for the intraobserver, interobserver, and intervisit variability tests for the OS/RPE junction oscillated between 17% and 63%.

The ICCs obtained for the intraobserver and intervisit variability tests were greater than 80% for the total retina, GCL+IPL, and ONL. The lowest ICC values were obtained for the interobserver variability test (see Table 9). The ICCs were also greater than 70% for the RNFL in R3, R4, R6, and R7 (intraobserver and interobserver variability test) and greater than 80% in R6 and R7 (intervisit variability test). The highest ICC values (>70%) for the OPL were obtained in R4, R5, and R7 (intraobserver variability test). The highest ICCs values (>70%) for the INL were obtained in R3 (interobserver and intervisit variability test) and R4 (intervisit variability test). The ICCs were also greater than 70% for the OS/RPE junction in R1, R3 (intraobserver variability test) and R4 (intraobserver and intervisit variability test). The Wilcoxon paired measurements test showed that there were no statistically significant differences between measurements obtained by different examiners or during different visits.

Discussion

The current Stratus OCT software provides a map of the macula and nerve fiber layer thickness along with regional averages, but it does not provide thickness measurements of the separate retinal layers. This limitation in the Stratus OCT system has stimulated interest in developing segmentation algorithms to better detect the local changes in the retinal structure to improve retinal disease detection and its progression. In this study, the regional reliability and reproducibility of macular segmentation mapping is reported using OCTRIMA. The OCTRIMA software overcomes the limitations of the Stratus OCT software and provides additional quantitative information that can be extracted from OCT data. An important aspect of any test's ability to detect change is the reproducibility and reliability of the measurements, which was investigated in this study. The OCTRIMA software provides both uninterpolated average thickness and regional thickness result data, which are useful measurements both in clinical studies and practice.

Regional reproducibility results for the RNFL showed the nasal regions (R3 and R7) to be the most reproducible (highest ICCs), whereas the temporal regions (R5 and R9) to be the least reproducible (see Table 9), with fairly good reproducibility in the superior and inferior regions. The reason for this is that—according to this study's observations and supported by histological data—the RNFL layer is often undetectable at the temporal part of the macula. In addition, the ICC value for the superior and inferior regions was smaller than the value for the nasal region. This is due to the smaller mean RNFL thickness values in the superior and inferior regions relative to the nasal part of the macula. The regional reproducibility results for the GCL+IPL complex showed the pericentral regions (R2-R5) to be more reproducible than the peripheral regions (R6-R9). It is possible that some of the variability encountered in the peripheral regions may be attributed to the fact that the number of measured points significantly decreases from the center to the periphery, where the tomograms are more spaced. The ICCs were particularly low for the INL and OPL, indicating that the combination of these layers could be a good alternative to minimize the variability of detected borders between them. The reproducibility results for the OS/RPE junction in all macular regions were the least reproducible with a relatively low ICC due to the low contrast between the OS/RPE junction (outer border) and the RPE inner boundary, which can be attributed to the limitation of the Stratus OCT system to penetrate deeper structures in the retina. The ICCs were particularly high for all 3 groups of comparisons for the total retina; GCL+IPL complex and the ONL (see Table 9).

The regional thickness measurements for the total retina showed that coefficients of repeatability ranged from 3 to 5% in healthy eyes, and coefficients of reproducibility ranged from 3 to 6% (interobserver variability) and 1 to 4% (intervisit variability). The enhanced repeatability and reproducibility coefficients observed here, are likely due to the robustness of OCTRIMA's segmentation algorithm. The improved ICCs of 85% to 98% achieved for the total retina are also comparable with the values previously reported (ICCs of 69% to 99%). Besides, lower repeatability and reproducibility coefficients were obtained for the total retina in the central region (i.e. R1) where large variations in retinal thickness occur due to the shape of the foveal depression (see Table 8). In addition, whichever observer or visit was considered, mean retinal thickness values in healthy subjects were very similar.

In general, the higher variability found in the peripheral regions may be attributed to relatively fewer sampled points. The intraobserver repeatability coefficient for the total retinal thickness was less than 7 μm for all regions, indicating that thickness changes of 14 μm can be detected over time with 95% confidence. Similarly, interobserver and intervisit reproducibility coefficients for the total retinal thickness were less than 9 μm and 6 μm for all regions, respectively; indicating that thickness abnormalities of 18 μm and 12 μm can be detected over time with 95% confidence. Furthermore, the intraobserver repeatability coefficient for intraretinal layers (except the GCL+IPL complex) was less than 4 μm (6 μm for GCL+IPL complex) for all regions, indicating that thickness changes of 8 μm (12 μm) can be detected over time with 95% confidence. Likewise, the intervisit reproducibility coefficient for intraretinal layers (except the GCL+IPL complex) was less than 4 μm (5 μm for the GCL+IPL complex) for all regions, indicating that thickness changes of 8 μm (10 μm) can be detected over time with 95% confidence. The results herein, also indicated a higher thickness of the RNFL and GCL+IPL complex within 1 mm nasal to the foveal center compared to all other regions due to an increase in the ganglion cell axon and nerve fiber layer thickness in this region. Similarly, the thickness of these layers minimized at the foveal center because of the anatomical structure of the fovea. Moreover, a higher thickness value was also obtained for the ONL at the fovea (i.e., R1) compared to all other regions due to the elongated foveal cone photoreceptors.

The results, herein, for the regional thickness analysis showed ICCs of 85-98% along with coefficients of repeatability and reproducibility less than 5 and 7%, respectively. In addition, intraobserver SDs of 1 to 6 μm, interobserver SDs of 5 to 8 μm, and intervisit SDs of 1 to 5 microns were obtained. Other groups have measured similar values for normal subjects. For example, Polito et al. (Arch Ophthalmol 2005; 123:1330-1337) obtained ICCs of 80-99%, SDs of 2 to 11 μm, along with coefficients of repeatability and reproducibility less than 8 and 10%, respectively. As with previous findings, change of examiner or grader did not significantly affect the reproducibility of the measurements in healthy eyes.

Conclusions

It is well known that repeatability and reproducibility of measurements is strictly dependant on how easily the optical cross sections can be consistently placed over the same points during each scan, how great the variation in retinal thickness along neighboring points is, and how many points are measured for each region. In this study, thickness measurements obtained for each of the 9 ETDRS like-regions are more repeatable and reproducible in the pericentral than in the peripheral ring. The INL, OPL, and OS/RPE junction were not very well reproducible in the regional analysis. Reproducibility could be improved for these layers by increasing the number of A-scans, which will produce an image with a high transverse pixel density, resulting in a better image quality. Conversely, this will increase the image acquisition time, which could lead to measurement errors due to eye motion. However, acquisition speed and resolution, which presented early challenges in OCT development, have experienced remarkable improvements with the introduction of a newer generation of OCT known as spectral domain OCT. These improvements are likely to partially afford the best solutions to the limitation of existing OCT software by providing images with higher resolution along with a dense map of the retina with precise registration and localization. Even though the results presented are based on Stratus OCT images, the main purpose was to establish the feasibility of our quantitative methodology for OCT image analysis independent of the technology used. OCTRIMA could facilitate a well defined and standardized quantitative analysis for the assessment of retinal diseases and its progression using OCT images obtained with Stratus OCT and SDOCT devices. This study was performed on a young group of healthy subjects (undilated eyes) and showed a high ICC for all three groups of comparisons for the total retina, the GCL+IPL complex and the ONL. Future studies will examine the repeatability and reproducibility of macular segmentation mapping with OCTRIMA in older healthy subjects and patients with early diabetic retinopathy and other retinal diseases.

TABLE 7
Mean thickness and standard deviation (SD) values for 1ten healthy eyes
obtained for each ETDRS region.

| | Scan 1 | Scan 2 | Scan 3 | Scan 4 |
|---|---|---|---|---|
| Layer | (E1D1S1) | (E1D1S2) | (E2D1S1) | (E1D2S1) |
| RNFL | | | | |
| R1 | NL | NL | NL | NL |
| R2 | 34.09 ± 2.23 | 33.51 ± 2.85 | 35.10 ± 3.76 | 34.92 ± 2.42 |
| R3 | 31.06 ± 3.57 | 31.34 ± 2.99 | 33.30 ± 5.02 | 33.26 ± 3.26 |
| R4 | 31.32 ± 2.21 | 32.4 ± 2.27 | 31.04 ± 2.12 | 31.09 ± 1.57 |
| R5 | 19.97 ± 1.58 | 20.96 ± 3.05 | 21.62 ± 2.86 | 20.39 ± 3.21 |
| R6 | 51.31 ± 4.33 | 50.99 ± 4.25 | 52.07 ± 3.99 | 51.39 ± 4.73 |
| R7 | 55.57 ± 4.96 | 56.39 ± 4.96 | 55.72 ± 5.28 | 56.77 ± 4.85 |
| R8 | 39.1 ± 2.53 | 39.28 ± 1.71 | 38.81 ± 1.93 | 39.36 ± 1.94 |
| R9 | 24.63 ± 0.91 | 25.84 ± 3.51 | 26.45 ± 3.95 | 25.74 ± 3.66 |
| GCL + IPL | | | | |
| R1 | NL | NL | NL | NL |
| R2 | 87.94 ± 10.62 | 87.85 ± 9.79 | 86.17 ± 11.07 | 87.19 ± 11.39 |
| R3 | 90.89 ± 10.24 | 90.5 ± 10.92 | 88.51 ± 11.86 | 90.51 ± 10.26 |
| R4 | 85.92 ± 9.16 | 84.75 ± 9.87 | 84.74 ± 11.39 | 85.8 ± 10.22 |
| R5 | 89.78 ± 7.99 | 87.97 ± 8.62 | 86.90 ± 11.24 | 89.36 ± 8.31 |
| R6 | 54.79 ± 8.17 | 54.52 ± 8.21 | 52.35 ± 9.58 | 54.63 ± 8.27 |
| R7 | 60.08 ± 9.92 | 60.6 ± 7.1 | 57.82 ± 10.76 | 59.77 ± 8.24 |
| R8 | 48.37 ± 7.88 | 47.29 ± 8.75 | 46.27 ± 7.57 | 49.17 ± 6.92 |
| R9 | 57.7 ± 8.46 | 55.53 ± 8.68 | 53.44 ± 9.06 | 57.28 ± 7.86 |
| INL | | | | |
| R1 | NL | NL | NL | NL |
| R2 | 36.5 ± 1.82 | 37.19 ± 2.22 | 36.62 ± 2.13 | 36.93 ± 2.1 |
| R3 | 36.52 ± 2.42 | 37.22 ± 1.93 | 37.01 ± 2.51 | 37.19 ± 2.69 |
| R4 | 36.8 ± 1.58 | 36.91 ± 1.36 | 37.41 ± 1.33 | 36.6 ± 1.66 |
| R5 | 34.15 ± 1.61 | 34.37 ± 1.88 | 34.70 ± 1.96 | 33.93 ± 2.43 |
| R6 | 28.89 ± 2.5 | 30.5 ± 3.18 | 29.77 ± 1.46 | 29.12 ± 1.65 |
| R7 | 31.25 ± 1.84 | 31.16 ± 4.02 | 31.85 ± 2.76 | 31.03 ± 2.71 |
| R8 | 29.84 ± 1.71 | 31.36 ± 2.4 | 31.51 ± 3.25 | 30.69 ± 2.69 |
| R9 | 29.6 ± 2.03 | 29.62 ± 2.26 | 30.81 ± 2.47 | 30.28 ± 1.28 |
| OPL | | | | |
| R1 | NL | NL | NL | NL |
| R2 | 33.72 ± 1.52 | 34.49 ± 1.99 | 33.54 ± 1.31 | 34.34 ± 1.46 |
| R3 | 33.85 ± 0.91 | 35.03 ± 2.06 | 34.12 ± 1.45 | 34.50 ± 1.77 |
| R4 | 33.83 ± 1.66 | 34.54 ± 1.88 | 34.07 ± 2.20 | 33.75 ± 1.13 |
| R5 | 32.62 ± 1.40 | 32.97 ± 1.47 | 32.90 ± 2.20 | 32.79 ± 2.18 |
| R6 | 29.22 ± 2.00 | 28.66 ± 1.55 | 28.27 ± 1.21 | 29.77 ± 2.39 |
| R7 | 29.31 ± 1.66 | 29.46 ± 1.60 | 29.21 ± 1.75 | 30.56 ± 3.21 |
| R8 | 29.24 ± 2.50 | 28.80 ± 2.04 | 29.74 ± 3.88 | 29.23 ± 2.00 |
| R9 | 28.71 ± 1.57 | 29.34 ± 0.96 | 30.20 ± 3.00 | 28.67 ± 1.41 |
| ONL | | | | |
| R1 | 113.21 ± 8.48 | 111.95 ± 8.15 | 111.37 ± 9.21 | 111.3 ± 8.47 |
| R2 | 88.24 ± 4.99 | 88.05 ± 5.34 | 87.51 ± 5.86 | 88.39 ± 4.05 |
| R3 | 89.87 ± 5.12 | 88.51 ± 5.43 | 88.80 ± 6.09 | 88.98 ± 4.55 |
| R4 | 84.31 ± 4.90 | 83.81 ± 4.79 | 83.21 ± 6.81 | 85.01 ± 4.92 |
| R5 | 88.73 ± 5.29 | 88.74 ± 5.52 | 88.63 ± 6.34 | 89.06 ± 5.86 |
| R6 | 75.14 ± 4.46 | 76.21 ± 5.48 | 75.01 ± 4.10 | 75.24 ± 4.96 |
| R7 | 73.34 ± 5.60 | 72.63 ± 5.54 | 73.76 ± 6.54 | 72.83 ± 6.04 |
| R8 | 71.08 ± 4.98 | 70.68 ± 5.63 | 70.68 ± 4.87 | 70.33 ± 3.43 |
| R9 | 74.80 ± 5.28 | 74 ± 4.49 | 74.98 ± 4.89 | 73.98 ± 4.70 |
| OS/RPE | | | | |
| R1 | 16.03 ± 1.72 | 15.95 ± 1.48 | 15.39 ± 1.66 | 15.49 ± 1.29 |
| R2 | 11.54 ± 1.83 | 10.74 ± 1.35 | 12.20 ± 3.47 | 11.53 ± 1.96 |
| R3 | 10.83 ± 1.88 | 10.82 ± 1.36 | 11.39 ± 1.88 | 11.66 ± 2.19 |
| R4 | 12.53 ± 2.53 | 12.63 ± 3.12 | 12.86 ± 3.52 | 12.19 ± 2.51 |
| R5 | 10.96 ± 1.72 | 12.26 ± 2.68 | 11.83 ± 2.29 | 11.60 ± 1.77 |
| R6 | 13.97 ± 2.72 | 12.13 ± 1.73 | 13.60 ± 2.93 | 12.91 ± 2.11 |
| R7 | 13.78 ± 1.77 | 12.53 ± 2.35 | 12.57 ± 2.17 | 13.91 ± 1.87 |
| R8 | 14.17 ± 3.14 | 13.48 ± 1.52 | 13.58 ± 2.57 | 13.33 ± 3.04 |
| R9 | 13.61 ± 2.06 | 12.48 ± 2.17 | 13.33 ± 2.08 | 11.82 ± 1.81 |
| Total | | | | |
| Retina | | | | |
| R1 | 215.09 ± 15.65 | 217.47 ± 15.8 | 219.34 ± 17.56 | 217 ± 18.25 |
| R2 | 311.5 ± 15.47 | 314.26 ± 17.63 | 311.58 ± 18.69 | 313.5 ± 17.19 |
| R3 | 313.26 ± 16.68 | 314.75 ± 17.53 | 315.23 ± 20.43 | 315.6 ± 18.23 |
| R4 | 304.13 ± 13.04 | 305.47 ± 15.04 | 303.49 ± 17.61 | 304.3 ± 15.72 |
| R5 | 298.20 ± 12.29 | 298.56 ± 12.89 | 299.08 ± 17.33 | 298.7 ± 14.10 |
| R6 | 272.49 ± 9.41 | 275.44 ± 12.67 | 271.72 ± 12.82 | 274.3 ± 10.66 |
| R7 | 281.49 ± 11.49 | 283.90 ± 12.31 | 282.78 ± 16.29 | 283 ± 13.24 |
| R8 | 250.71 ± 9.02 | 251.90 ± 10.85 | 251.37 ± 10.16 | 252.4 ± 10.47 |
| R9 | 248.8 ± 11.79 | 248.75 ± 12.36 | 250.73 ± 13.54 | 250.4 ± 10.76 |

Values shown were obtained for the total retina and six intraretinal layers during each scanning session.
E1D1S1 is the first set of scans acquired by Examiner 1 on Day 1 during Session 1;
E1D1S2 is the second set of scans acquired by Examiner 1 on Day 1 during Session 2;
E2D1S1 is the third set of scans acquired by Examiner 2 on Day 1 during Session 1 and
E1D2S1 is the fourth set of scans acquired by Examiner 1 on Day 2 during Session 1.
R1 to R9 represent the 9 ETDRS-like regions of the macula.
NL indicates “No Layer” present in the region.
The mean thickness and SD values are reported in micrometers.

TABLE 8
Coefficients of repeatability and reproducibility obtained for each
intraretinal layer thickness and the total retinal thickness.
Coefficients of Repeatability (Intraobserver Variability) (%)

| E1D1S1- | | GCL + | | | | OS/ | Total |
|---|---|---|---|---|---|---|---|
| E1D1S2 | RNFL | IPL | INL | OPL | ONL | RPE | Retina |
| R1 | NL | NL | NL | NL | 2.54 | 24.33 | 4.75 |
| R2 | 10.50 | 7.64 | 10.85 | 11.28 | 4.23 | 17.32 | 3.32 |
| R3 | 8.31 | 4.79 | 11.94 | 11.31 | 7.10 | 22.13 | 3.94 |
| R4 | 16.06 | 7.91 | 8.09 | 6.71 | 5.30 | 40.74 | 3.96 |
| R5 | 23.80 | 8.61 | 13.61 | 10.05 | 6.90 | 53.02 | 3.30 |
| R6 | 10.52 | 20.34 | 22.19 | 8.99 | 5.75 | 32.62 | 3.22 |
| R7 | 10.95 | 11.47 | 14.76 | 21.46 | 5.53 | 33.04 | 3.04 |
| R8 | 16.16 | 17.24 | 12.23 | 11.42 | 5.68 | 30.12 | 3.13 |
| R9 | 24.64 | 15.14 | 13.39 | 10.54 | 5.81 | 31.37 | 3.34 |

Coefficients of Reproducibility (Interobserver Variability) (%)

| E1D1S1- | | GCL + | | | | OS/ | Total |
|---|---|---|---|---|---|---|---|
| E2D1S1 | RNFL | IPL | INL | OPL | ONL | RPE | Retina |
| R1 | NL | NL | NL | NL | 5.36 | 43.50 | 6.14 |
| R2 | 18.09 | 7.21 | 5.65 | 11.65 | 7.49 | 48.41 | 4.96 |
| R3 | 9.12 | 6.77 | 8.81 | 11.44 | 10.67 | 62.84 | 4.36 |
| R4 | 18.00 | 9.87 | 6.93 | 9.85 | 7.52 | 36.85 | 5.44 |
| R5 | 19.51 | 10.45 | 13.59 | 14.54 | 7.25 | 46.76 | 3.42 |
| R6 | 7.65 | 16.41 | 17.24 | 14.68 | 10.44 | 42.27 | 5.69 |
| R7 | 7.13 | 21.74 | 22.55 | 29.95 | 14.53 | 57.49 | 3.79 |
| R8 | 19.61 | 22.25 | 15.65 | 22.84 | 7.29 | 42.87 | 4.19 |
| R9 | 29.36 | 20.54 | 15.07 | 20.24 | 7.06 | 36.01 | 5.32 |

Coefficients of Reproducibility (Intervisit Variability) (%)

| E1D1S1- | | GCL + | | | | OS/ | Total |
|---|---|---|---|---|---|---|---|
| E1D2S1 | RNFL | IPL | INL | OPL | ONL | RPE | Retina |
| R1 | NL | NL | NL | NL | 4.69 | 26.59 | 4.35 |
| R2 | 11.31 | 6.07 | 10.03 | 13.41 | 5.49 | 39.11 | 3.16 |
| R3 | 12.02 | 4.65 | 7.45 | 8.64 | 7.45 | 30.64 | 3.26 |
| R4 | 18.82 | 8.17 | 9.45 | 10.70 | 6.35 | 28.45 | 3.35 |
| R5 | 22.85 | 5.31 | 14.01 | 17.32 | 4.13 | 30.79 | 3.46 |
| R6 | 9.11 | 12.15 | 16.88 | 13.44 | 5.58 | 27.31 | 3.26 |
| R7 | 8.23 | 13.80 | 16.40 | 19.20 | 5.97 | 55.05 | 1.37 |
| R8 | 16.47 | 16.07 | 4.89 | 13.47 | 5.89 | 36.10 | 1.35 |
| R9 | 25.05 | 13.92 | 9.38 | 13.63 | 5.71 | 32.33 | 1.51 |

Values are provided for each ETDRS region.

TABLE 9
ICCs obtained for the total retina and 6 intraretinal layers.

| | Intraobserver | Interobserver | Intervisit |
|---|---|---|---|
| | (E1D1S1- | (E1D1S1- | (E1D1S1- |
| Regions | E1D1S2) | E2D1S1) | E1D2S1) |
| Total | | | |
| Retina | | | |
| R1 | 0.94 | 0.87 | 0.95 |
| R2 | 0.94 | 0.91 | 0.95 |
| R3 | 0.95 | 0.92 | 0.95 |
| R4 | 0.91 | 0.90 | 0.94 |
| R5 | 0.88 | 0.85 | 0.94 |
| R6 | 0.90 | 0.89 | 0.86 |
| R7 | 0.91 | 0.88 | 0.94 |
| R8 | 0.90 | 0.88 | 0.97 |
| R9 | 0.94 | 0.86 | 0.98 |
| RNFL | | | |
| R1 | NL | NL | NL |
| R2 | 0.58 | 0.63 | 0.72 |
| R3 | 0.88 | 0.70 | 0.69 |
| R4 | 0.72 | 0.71 | 0.68 |
| R5 | 0.30 | 0.23 | 0.35 |
| R6 | 0.74 | 0.87 | 0.86 |
| R7 | 0.85 | 0.93 | 0.86 |
| R8 | 0.46 | 0.59 | 0.57 |
| R9 | 0.12 | −0.05 | 0.18 |
| GCL + IPL | | | |
| R1 | NL | NL | NL |
| R2 | 0.95 | 0.93 | 0.95 |
| R3 | 0.96 | 0.94 | 0.98 |
| R4 | 0.98 | 0.94 | 0.98 |
| R5 | 0.90 | 0.88 | 0.91 |
| R6 | 0.80 | 0.72 | 0.92 |
| R7 | 0.84 | 0.93 | 0.95 |
| R8 | 0.92 | 0.59 | 0.87 |
| R9 | 0.85 | 0.68 | 0.89 |
| INL | | | |
| R1 | NL | NL | NL |
| R2 | 0.27 | 0.53 | 0.54 |
| R3 | 0.60 | 0.89 | 0.74 |
| R4 | 0.05 | 0.47 | 0.70 |
| R5 | 0.62 | 0.38 | 0.62 |
| R6 | 0.56 | 0.21 | 0.17 |
| R7 | 0.52 | 0.43 | 0.60 |
| R8 | 0.15 | −0.09 | 0.34 |
| R9 | 0.59 | 0.28 | 0.61 |
| OPL | | | |
| R1 | NL | NL | NL |
| R2 | 0.19 | 0.35 | −0.04 |
| R3 | −0.07 | 0.43 | −0.12 |
| R4 | 0.71 | 0.53 | 0.56 |
| R5 | 0.78 | 0.63 | 0.52 |
| R6 | 0.50 | −0.03 | 0.07 |
| R7 | 0.75 | 0.34 | 0.64 |
| R8 | 0.03 | 0.05 | 0.22 |
| R9 | 0.24 | 0.13 | 0.16 |
| ONL | | | |
| R1 | 0.93 | 0.89 | 0.92 |
| R2 | 0.92 | 0.87 | 0.89 |
| R3 | 0.84 | 0.78 | 0.81 |
| R4 | 0.91 | 0.70 | 0.87 |
| R5 | 0.93 | 0.83 | 0.87 |
| R6 | 0.81 | 0.74 | 0.93 |
| R7 | 0.94 | 0.78 | 0.92 |
| R8 | 0.92 | 0.43 | 0.88 |
| R9 | 0.90 | 0.88 | 0.90 |
| OS/RPE | | | |
| R1 | 0.76 | −0.11 | 0.31 |
| R2 | 0.01 | 0.20 | 0.43 |
| R3 | 0.85 | −0.27 | 0.34 |
| R4 | 0.87 | 0.37 | 0.79 |
| R5 | 0.28 | 0.30 | 0.47 |
| R6 | −0.01 | 0.45 | 0.64 |
| R7 | 0.44 | −0.15 | 0.61 |
| R8 | 0.49 | 0.00 | 0.13 |
| R9 | 0.45 | 0.34 | 0.18 |

### Example 10

### Improving Image Segmentation Performance and Quantitative Analysis Via a Software Tool for OCT Retinal Image Analysis

Materials and Methods

Data collection to be studied: The study conducted in this paper followed the Health Insurance Portability and Accountability Act-compliant and was approved by the Institutional Review Board in our institutions. The research adhered to the tenets set forth in the declaration of Helsinki. A preexisting report and image repository in our institutions was retrospectively searched for individuals of all ages and both sexes who underwent Stratus OCT imaging of the macula. All study cases were obtained between Jan. 1, 2002, and Dec. 10, 2008, using the radial lines protocol (1024 samples×512 A-scans per B-scan) on a single Stratus OCT instrument (version 4.0 software). Complementary cases with clinically significant intraretinal features from Stratus OCT and SDOCT systems were also collected, for demonstration purposes only. In addition, to demonstrate intragrader and intergarder reproducibility of OCTRIMA and the comparability of STRATUS and OCTRIMA-derived regional thickness results, the same set of ten healthy eyes were used from five normal subjects as reported previously (D. Cabrera DeBuc, et al., J. Biomed. Opt, 14(6) (November/December 2009)) ranging in age from 25 to 34 yr (mean age 29 yr). All subjects underwent visual acuity testing with refraction and a complete slit-lamp examination. Informed consent was obtained from each subject.

Macular radial line scans of the retina for each case were exported to disc with the export feature available in the Stratus OCT device, and total retinal thickness measurements were obtained as provided by the Stratus OCT built-in algorithms as well as with the OCTRIMA software. OCTRIMA's measurements are obtained by calculating the thickness between the inner limiting membranr (ILM) and the inner boundary of the second hyperreflective band corresponding to the inner surface of the outer segment/retinal pigment epithelium (OS/RPE) junction; whereas Stratus OCT measurements are calculated between ILM and the inner boundary of the innermost hyperreflective band corresponding to the inner segment-outer segment (IS/OS) border. Stratus OCT retinal thickness measurements were compared with the OCTRIMA measurements in each of the nine macular regions defined by the Early Treatment Diabetic Retinopathy Study (ETDRS). Differences in the measurements of the macular volume measurements were also calculated. Moreover, the agreement of the thickness measured by OCTRIMA and Stratus OCT and those recently obtained with Fourier-domain OCT (FD-OCT) systems were also evaluated.

OCTRIMA system: OCTRIMA was developed using the Matlab graphical user interface design environment (GUIDE) tool that allows interactive design of a graphical window along with variables and commands linked to it (The Mathworks, Natick, Mass.). Specifically, OCTRIMA is a research software application package that integrates an automated and semiautomated segmentation algorithm along with the manual correction tool into a user-friendly graphical user interface (GUI). The OCTRIMA software originally written in Matlab code is converted into C and requires the MatLab Component Runtime (MCR), which must also be installed on the end-user's computer. It also requires a PC with the WINDOWS™ operating system installed (Microsoft Corp., Redmond, Wash.). The application essentially provides dual functionality in a single software package by combining image enhancement and speckle denoising of Stratus OCT images along with intraretinal segmentation and error correction using direct visual evaluation of the detected boundaries. Moreover, the software has the capability to perform calculations based on measured values of corrected thickness and reflectance of the various cellular layers of the retina and the whole macula.

The user interface of OCTRIMA is mainly organized into panels that group GUI components and make the GUI easier to understand by visually grouping related controls. The GUI guides the user in an organized manner by activating only relevant functions and disabling other functions at any given time, achieved by effectively using the Matlab handles structure.

The input of the OCTRIMA software simply consists of two types of data files: 1) the data file with the patient information (i.e., a text file exported from the Stratus OCT system) and 2) the raw scan data (i.e., the image file). In addition, OCTRIMA facilitates the analysis of OCT images from two OCT scanning protocols: regular high-resolution (1024×512) and fast low-density mode (128×512). In addition, the file, view, data and help menus provide various options for file management, image data and segmentation results viewing, image processing and quantitative analysis, generating and exporting the results in various format and comprehensive visual help. Moreover, multiple OCT B-scans can be loaded at once and viewed one at a time by using the review option in the File menu. OCTRIMA also include functions that use custom built algorithms developed for generating the report for quantitative analysis, exporting the numerical results (i.e., thickness and reflectance data) to a MS Excel spreadsheet and generating topographic and fractional loss maps for the retinal thickness of the overall macula and each intraretinal layer. In addition, a final report in PDF format can be also generated.

Automated segmentation module: In the OCTRIMA startup screen in the automated mode, the general information panel displays the name of the displayed OCT B-scan and its saved segmentation results as well as important subject and OCT B-scan information such as left eye (OS) or right eye (OD), scan date, the scan angle orientation (in degrees) and its pictorial representation. The “Control Panel” in automatic mode facilitates the following functionalities: 1. Preprocessing of OCT raw data. The filtering of the speckle noise is performed during the preprocessing step. The preprocessing algorithm parameters are a set of invariant numerical values that already have been optimized and hence it is encapsulated and invisible to the user. 2. Automated segmentation of the various cellular layers of the retina. Segmentation is achieved by finding peaks on each sampling line using the structure coherence matrix. A total of eight intraretinal boundaries are automatically detected while the outer boundary of the IS/OS and a Choriocapillaris section are assumed at fixed distances using anatomical knowledge. Thus, a total of ten boundaries and seven intraretinal layers are extracted. More details of the segmentation process can be found in Cabrera et al. (Opt. Express 13, 10200-10216 (2005)). 3. Semiautomatic correction of discontinuities in each detected boundary after automated segmentation.

Manual correction module: Segmentation of the object of interest is considered a difficult step in the analysis of medical images. Fully automatic methods sometimes fail, producing incorrect results and requiring the intervention of a human operator. This is often true in ophthalmic applications such as OCT where image segmentation is particularly difficult due to restrictions imposed by image acquisition, ocular pathology and biological variation. Consequently, the intervention of a human operator is often needed to correct the segmentation result manually. Strategies that allow a trained human expert to correct segmentation errors may provide a suitable mechanism for increasing the precision of retinal measurements for monitoring patients with macular disease, particularly in clinical trials.

Computer aided manual correction of OCT segmentation may be useful for correcting thickness measurements in cases with errors of automated retinal boundary detection and may also be useful for quantitative analysis of clinically relevant features, such as the volume of subretinal fluid and intraretinal fluid-filled regions. For example, it is well known that detection algorithms fail when the retinal structure is disrupted by fluid accumulation which can lead to inaccurate measurements of retinal thickness. Thus, there is a need for developing efficient, user-friendly software tools that will supplement accurate automated boundary detection algorithms to generate more precise segmentation of the various cellular layers of the retina. For instance, an interactive procedure could be activated, by means of which the user edits the segmentation directly or provides extra information to reconfigure the computational part. If the result generated by the computational part is wrong, the user can correct it directly using a manual editor.

In OCTRIMA, various manual corrective functions are implemented to assist the user in correcting retinal segmentation errors resulting from the automated and semiautomated segmentation process. These errors are mainly due to both the presence of high reflectivity regions in the inner retina and loss of retinal structure information in local regions along the retinal cross-section as visualized by the commercial OCT system. All the functions and algorithms used in the manual correction process display the delineation of the retinal boundaries on the screen, enabling the user to instantly evaluate its location on the image grid. Different visualization schemes are adopted to show the detected layers as well as the corrections in color for better discrimination from the grey image in the background.

Additional OCTRIMA-based measures: OCTRIMA also offers objective and intuitive additional functions for evaluating and comparing the efficacy of different therapeutic modalities. Since normative data for OCT analysis are crucial to compare various treatment strategies, OCTRIMA facilitates normative data from healthy controls and also allows the user to generate a new norm using healthy or pathological subjects. The OCTRIMA's norm is based on data from 74 healthy subjects (35±13 yr).

OCTRIMA also provides an indicator to evaluate the quality of the OCT B-scans. Generally, the standard deviation of the foveal center point thickness is used as a measure of the scan variance. A high standard deviation (>10% of center point thickness) indicates high variability, usually due to patient movement or boundary line error, and therefore incorrect center point thickness. Good quality images have a standard deviation <10% of center point, good clarity of the layers and are also well centered. Substantially decentered scans could have a low standard deviation. Thus, a scan quality factor (SQF) based on the standard deviation calculation (in %) of the foveal center point (FCP) for the six radial line scans is included in the OCTRIMA software. A good scan has a SQF=1, indicating that the percentage standard deviation of the foveal center point is ≦10. In addition, OCTRIMA provides a standardized method for reporting changes in thickness as a percentage of total possible change based on normative OCT data. The “Calculate standardized thickness change” function calculates the total percentage change observed in the patient using the segmentation results before and after treatment.

Statistical methods: The coefficients of reproducibility were calculated using the methods outlined by Bland and Altman for each of the averaged thickness measurements obtained for the total retina and intraretinal layers. The coefficients of reproducibility were computed from the standard deviations (SDs) of the differences between measurements made by each grader. The statistical analysis was performed using the software package SPSS version 16 (SPSS Inc, Chicago, Ill.).

Results

Comparison of Stratus OCT thickness measurements and OCTRIMA thickness measurements: In contrast to Stratus OCT thickness calculations, OCTRIMA measurements of retinal thickness were obtained using the inner surface of the second hyperreflective band, that is assumed to be the OS/RPE junction. Table 10 shows the level of agreement between OCTRIMA and Stratus OCT when the two methods are applied on the same Stratus OCT images. Pearson correlation coefficients demonstrated R^{2>0.98} for all ETDRS regions. Since the calculations of OCTRIMA use the inner surface of the second hyperreflective band (i.e., OS/RPE junction) as the outer retinal border, the mean thickness difference corresponded to 11% of the measured Stratus OCT retinal thickness. These thickness differences were consistent with those obtained by comparing retinal thickness measurements between Stratus OCT and FD-OCT systems. Note that the mean difference for the fovea (R1) included only 17% of the measured value obtained by the Stratus OCT system. The mean difference results for the superior, nasal, inferior and temporal inner and outer regions of the macula (R2-R9) included 8-12% of the Stratus OCT measurements (see Table 10). Total macular volume, a measure derived from thickness in all data points of the macula, was 10% higher by OCTRIMA compared to Stratus OCT results, also supporting an average difference of 10% in thickness measurements.

TABLE 10
Comparison between Stratus OCT retinal thickness measurements and the OCTRIMA
measurements obtained using the inner border of the OS/RPE junction as the outer retinal
boundary (i.e., the inner boundary of the RPE). Differences in the measurement of the
total macular volume are also included and are expressed in cubic millimeters (mm³).

| | | | | Percent of the |
|---|---|---|---|---|
| | | | Mean Absolute | STRATUS OCT |
| | Stratus OCT | OCTRIMA | Difference | measurement |
| Macular Regions | (μm) | (μm) | (μm) | (%) |
| Fovea (R1) | 184.50 | 215.09 | 30.59 | 16.58 |
| Superior Inner (R2) | 283.90 | 311.50 | 27.60 | 9.72 |
| Nasal Inner (R3) | 281.00 | 313.26 | 32.26 | 11.48 |
| Inferior Inner (R4) | 280.70 | 304.13 | 23.43 | 8.35 |
| Temporal Inner (R5) | 266.20 | 298.20 | 32.00 | 12.02 |
| Superior Outer (R6) | 243.30 | 272.49 | 29.19 | 12.00 |
| Nasal Outer (R7) | 259.00 | 281.49 | 22.49 | 8.68 |
| Inferior Outer (R8) | 232.30 | 250.71 | 18.41 | 7.92 |
| Temporal Outer | 224.40 | 248.80 | 24.40 | 10.87 |
| (R9) | | | | |
| Mean | 250.59 | 277.30 | 26.71 | 10.85 |
| Range | (184.50-283.90) | (215.09-313.26) | (18.41-32.00) | (8.35-16.58) |

Total macular volume (mm³)

| | | | |
|---|---|---|---|
| 6.99 | 7.68 | 0.69 | 9.93 |

Intragrader and intergrader reproducibility of thickness measurements: As a result of scanning a total of ten healthy eyes, a total of 60 OCT B-scans were collected and analyzed by two independent experienced graders (G1, G2). Moreover, to assess the overall performance of the OCTRIMA software, the average (between the two graders) retinal thickness in each of the nine ETDRS regions obtained by OCTRIMA analysis was compared with the automated Stratus OCT results. All scans in the study had a signal strength of 9 or 10. Algorithm performance was visually evaluated by the experienced graders to detect segmentation errors. Criteria for algorithm error included evident disruption of the detected boundary (e.g. small peaks, linear and curve offsets), and/or detected boundary jumping to and from different anatomical structures (i.e. false segmentation). The average number of manual corrections needed per scan was three. The INL and OPL were the layers that required most of the manual corrections.

Table 11 shows the reproducibility attained by one grader (G2) after analyzing each of the ten eyes at two separate times (intragrader test, one week interval between analyses) using OCTRIMA software. Thickness measurements (mean±SD) of the total retina and intraretinal layers are also shown in Table 11. The coefficient of reproducibility (CR) obtained for the thickness measurements was less than 0.2% for the total retina, less than 0.4% for the ONL and less than 3% for the remaining layers. Overall, the median of the thickness differences as a percentage of the mean thickness was less than 1%. According to the results, the measurement accuracy of the OCTRIMA algorithm ranged between 0.27-1.47 μm (see Table 11). Excellent intragrader agreement can be observed in the Bland-Altman plots of the mean difference between both grading sessions for each of the calculated intraretinal layer thickness.

TABLE 11
Intragrader reproducibility using OCTRIMA.

| | Mean | Mean | *Mean | **Mean | ^{†}Median of | | |
|---|---|---|---|---|---|---|---|
| | Thickness | Thickness | Thickness ± | Absolute | Difference/ | | |
| | (1^{st} Grading) | (2^{nd} Grading) | SD | Difference | Mean Thickness | CR | CR |
| | (μm) | (μm) | (μm) | (μm) | (%) | (μm) | (%) |
| RNFL | 40.76 | 40.76 | 40.76 ± 1.39 | 0.17 | 0.22 | 0.47 | 1.16 |
| GCL + IPL | 72.65 | 72.79 | 72.72 ± 7.14 | 0.43 | 0.43 | 1.47 | 2.03 |
| INL | 34.30 | 34.50 | 34.40 ± 1.25 | 0.25 | 0.56 | 0.50 | 1.45 |
| OPL | 32.64 | 32.62 | 32.63 ± 0.62 | 0.22 | 0.52 | 0.57 | 1.76 |
| ONL | 88.37 | 88.35 | 88.36 ± 4.98 | 0.10 | 0.08 | 0.27 | 0.30 |
| Total Retina | 280.50 | 280.63 | 280.56 ± 12.39 | 0.21 | 0.07 | 0.46 | 0.17 |

*Mean thickness value averaged across all 10 eyes. The mean thickness value is a result of averaging the uninterpolated thickness measurements at every A-scan location for all six B-scans of all 10 eyes.
**This value is calculated by subtracting OCTRIMA thickness measurement obtained during the 1st grading from the OCTRIMA thickness measurement obtained during the 2nd grading for each eye by the same grader G2, and then taking the absolute value.
^{†}Median of the differences between measurements expressed as a percentage of mean thickness across all 10 eyes.

Table 12 shows the level of agreement between the two graders (intergrader reproducibility test, i.e. G1 versus G2) using the OCTRIMA software. The coefficient of reproducibility obtained for the thickness measurements was less than 0.5% for the total retina, less than 0.7% for the ONL and less than 5% for the remaining layers. According to the results the measurement accuracy of our algorithm ranged between 0.6-1.76 μm (see Table 12). Overall, the median of the thickness differences as a percentage of the mean thickness was less than 2%

TABLE 12
Intergrader reproducibility using OCTRIMA

| | Mean | Mean | *Mean | **Mean | ^{†}Median of | | |
|---|---|---|---|---|---|---|---|
| | Thickness | Thickness | Thickness ± | Absolute | Difference/ | | |
| | (Grader 1) | (Grader 2) | SD | Difference | Mean Thickness | CR | CR |
| | (μm) | (μm) | (μm) | (μm) | (%) | (μm) | (%) |
| RNFL | 40.76 | 40.72 | 40.74 ± 1.62 | 0.67 | 1.55 | 1.70 | 4.16 |
| GCL + IPL | 72.65 | 73.63 | 73.14 ± 7.34 | 1.11 | 1.50 | 1.76 | 2.40 |
| INL | 34.30 | 33.91 | 34.11 ± 1.14 | 0.56 | 1.17 | 1.11 | 3.27 |
| OPL | 32.64 | 32.24 | 32.44 ± 0.66 | 0.48 | 1.47 | 0.84 | 2.58 |
| ONL | 88.37 | 88.56 | 88.46 ± 4.93 | 0.26 | 0.22 | 0.60 | 0.68 |
| Total Retina | 280.50 | 280.50 | 280.51 ± 12.26 | 0.49 | 0.15 | 1.24 | 0.44 |

*Mean thickness value averaged across all 10 eyes. The mean thickness value is a result of averaging the uninterpolated thickness measurements at every A-scan location for all six B-scans of all 10 eyes.
**This value is calculated by subtracting OCTRIMA thickness measurement obtained during the 1st grading from the OCTRIMA thickness measurement obtained during the 2nd grading for each eye by the same grader G2, and then taking the absolute value.
^{†}Median of the differences between measurements expressed as a percentage of mean thickness across all 10 eyes.

Image segmentation performance and error correction using OCTRIMA: Illustrative cases of diseases with subretinal anomalies and representative intraretinal boundary detection errors are shown in FIGS. 3A-3F.

Mild non-proliferative diabetic retinopathy without macular edema: FIGS. 3A-3F shows an OCTRIMA segmented B-scan before and after applying manual corrections. In this study case, the representative B-scan was taken from a set of images obtained for a diabetic patient with mild non-proliferative diabetic retinopathy without macular edema (male, 59 yr old). Characteristic intraretinal boundary detection errors such as small peaks, linear offsets, curve offsets and false segmentations are illustrated in FIG. 3A. False segmentation refers to the falsely detected inner and/or outer boundaries of an intraretinal layer. This particular error is most commonly found during the RNFL's outer border detection. Specifically, there are certain cases in which the true anatomical thickness of the RNFL layer (or some regions of the RNFL layer) might be negligible. In other cases, one side of the RNFL layer is completely invisible in the OCT image, like for example in the temporal part of a horizontal B-scan (see FIG. 3B). In such cases, a correction is required to overlap the inner and outer boundaries of the RNFL layer in the regions of negligible thickness (see FIGS. 3C and 3D). However, sometimes the boundary detection algorithm fails in such specific cases when localized bright spots of high intensity appear on some regions of the RNFL layer; and falsely displays the outer boundary of the RNFL layer as a result of the peak search algorithm which looks for zero crossings in the structure. Hence, the RNFL outer boundary must be manually corrected to appear overlap the inner boundary in the invisible part of the layer. As can be seen in FIG. 3B, the ILM boundary on the inner side of the RNFL is detected but no boundary is detected on the outer left side since the RNFL is not visible on this (temporal) side for this particular scan, whereas the RNFL is bright and clearly visible on the right (nasal) side of the scan (see FIGS. 3A and 3B).

FIG. 3C shows the manually corrected outer boundary of the IPL (outlined in yellow) using the “Small Peak” corrective function of the manual correction software tool which removes the overshoots or undershoots in the individual boundaries.

These are also parts of a boundary that form a straight line segment but are incorrectly detected as a peak or an elevated or depressed line segment by the automated segmentation algorithm. This detection error is classified as a linear offset. To resolve this class of error, the user has to manually select two points to draw a straight line segment on the specific boundary containing the offset. For example, a straight line segment was manually drawn to correct the linear offset in the outer boundary of the outer plexiform layer (OPL, see the boundary outlined in green in FIG. 3E. FIG. 3F shows the manual corrections for the inner and outer boundaries of the inner nuclear layer (INL, outlined in yellow and cyan, respectively) which had segmentation errors as a result of curve offsets (see FIG. 3A). Curve offset is a term given to the curved portion of a boundary that has not been recognized as a curve, instead, has been incorrectly segmented as an elevated or depressed curve. Curve offsets have been rectified using a function based on a customized contour model which was originally introduced to identify non-convex shapes in OCT images. This function allows the user to select multiple closely spaced points that will be joined to trace a curve and remove the offset. FIG. 3F shows the result of the “Curve Plotting” function applied to correct the inner and outer boundaries of the INL (outlined in yellow and cyan, respectively).

Additionally, an OCTRIMA predefined control is also in place for the inner retinal layers (RNFL, ganglion cell and inner plexiform layer complex (GCL+IPL) and INL) and outer plexiform layer (OPL) in a 1.5 mm diameter zone in the fovea, where retinal reflections are minimally visible. The control forces the ILM, the inner and outer side of the GCL+IPL complex, and the outer side of the INL and OPL to be coincident in this region (see FIG. 3A). Sometimes, small peaks appear at the periphery of this controlled foveal region. In such a case, it appears that the coincident layers deviate from the true foveal visible boundary and need to be corrected so that they overlap in the periphery of the controlled foveal region. Thus, the “Overlap” function of the manual correction software tool is useful to rectify the segmentation at the fovea (see FIG. 3D).

OCTRIMA thickness maps for the overall macula and each intraretinal layer obtained from the patient image data shown in FIGS. 3A-3F are as follows. The ETDRS-like regions are based on nine sectional thickness values in three concentric circles obtained from interpolation of the six linear scans, with diameters of 1, 3 and 6 mm. These maps are obtained according to the standards set by the ETDRS similarly to the Stratus OCT analysis software and can be easily exported to a PDF document along with the numerical results in tabulated format. The output data includes three main quantitative measures: thickness, volume and reflectance. The sectional measurements in the retinal thickness map are calculated from the averaged data from the six individual scans. The norm used in OCTRIMA was obtained from 74 healthy eyes (35±13 yr).

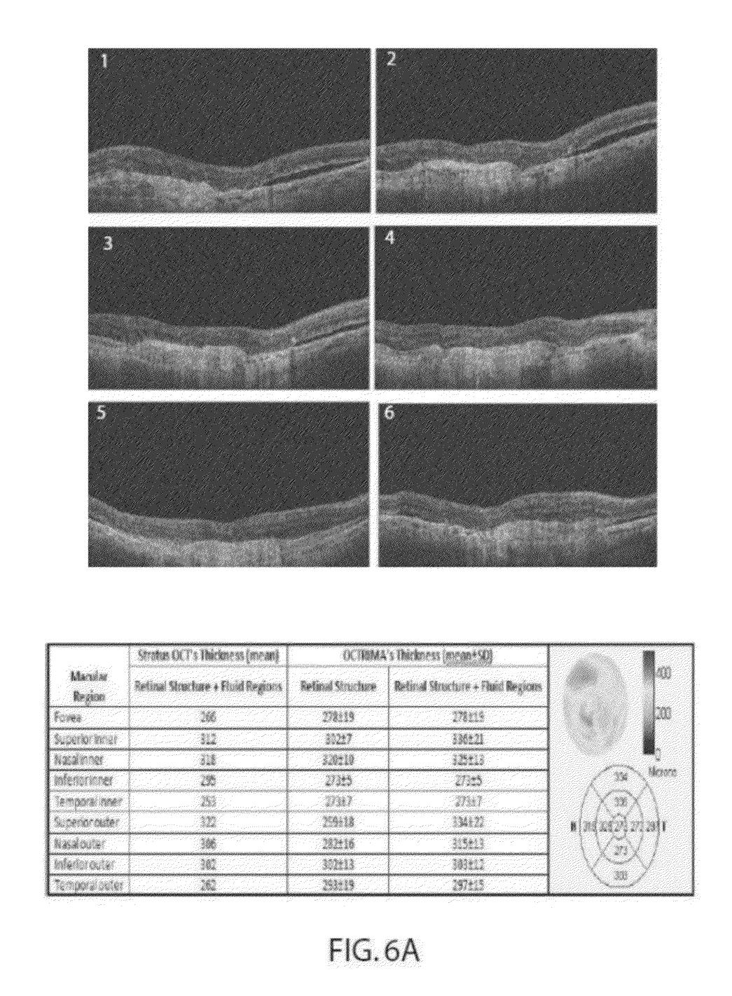

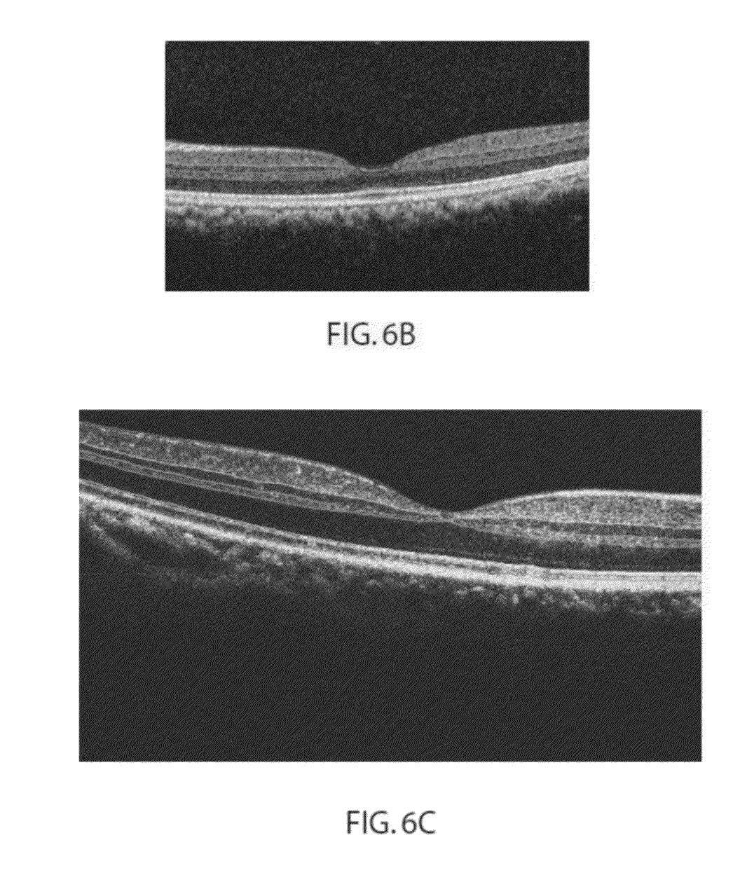

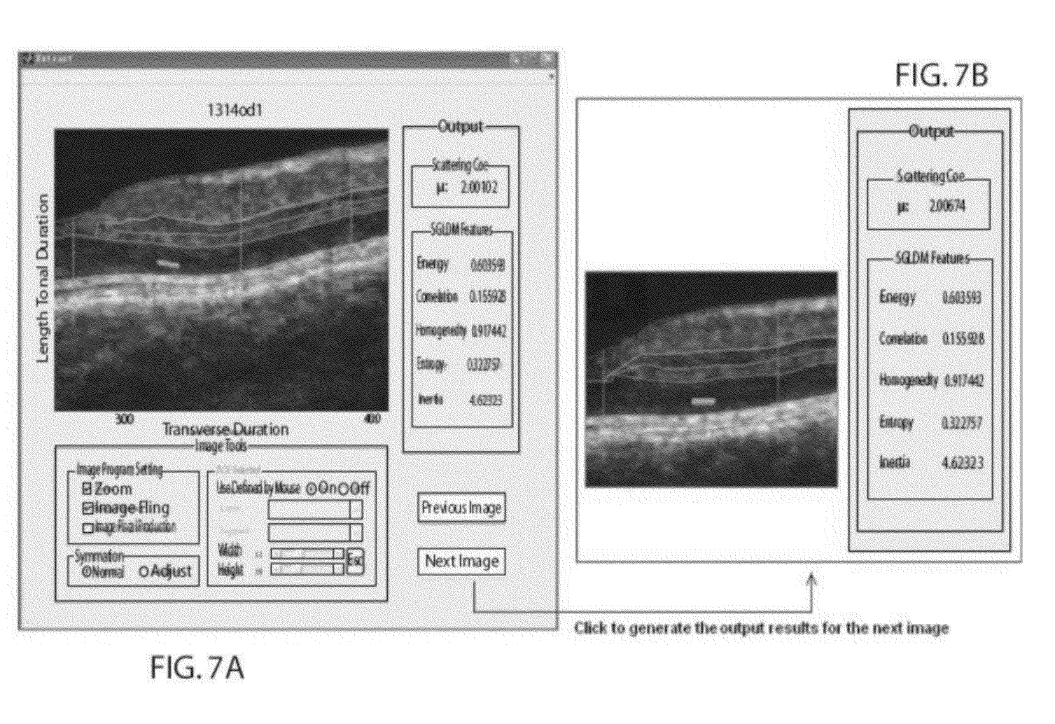

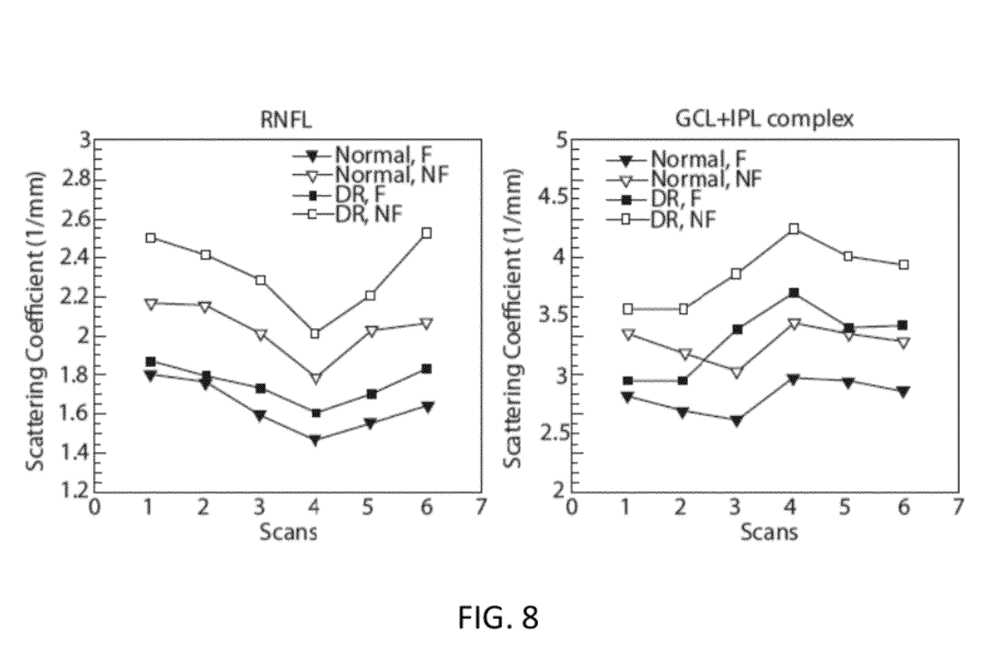

Neovascular age-related macular degeneration: It is well known that the current standard algorithms for obtaining the retinal thickness and volume information are error-prone when used to evaluate edematous retina and are unable to independently assess fluid under the retina and the retinal pigment epithelium. Even when the current algorithm accurately identifies the appropriate boundaries, the retinal volume and thickness calculations only take into account the entire area between the outer reflective band (retinal pigment epithelial layer) and the inner retinal surface. Thus, the algorithms are unable to independently assess the area and volume of the fluid filled cystic areas within and under the retina that represents leakage from choroidal neovascularization (CNV). However, OCTRIMA allows the user to trace the internal boundary of visible nonconvex shaped structures such as intraretinal and subretinal fluid-filled regions, if present and visible on the OCT B-scan. To quantify the area of these fluid-filled regions, an active contour model was used to outline these regions. Common errors occur in retinal boundary detection by the Stratus OCT software, for example, in scans obtained from a patient with neovascular age-related macular degeneration. In neovascular AMD, the fluid accumulation usually can be identified within the retina, under the retina, and under the RPE layer. In this case, peripheral and pericentral fluid-filled regions were observed in the OCT B-scans at presentation. Note that the Stratus algorithm erroneously detected the border of the innermost hyperreflective band in four of the six radial line scans. In addition, the fluid-filled region is included as part of the retinal structure for the thickness calculation. As a result, the retina appears thickened in this patient. OCTRIMA was able to correctly detect the boundaries of the retinal structure and the fluid-filled regions (see, FIG. 6A).

Advanced OCT imaging systems: Although the OCTRIMA quantitative analysis of Stratus OCT images described in this study is potentially useful, new OCT technologies, such as spectral domain, ultrahigh resolution and adaptive optics based OCT technology, are likely to partially afford better solutions to the limitation of existing OCT software by providing images with higher resolution along with a dense map of the retina with precise registration and localization. However, automatic segmentation algorithms for OCT data have tendency to give erroneous segmentation results especially in pathological cases, which is actually a result of the algorithm performance independently of how good the OCT image could be reproduced with a high level of detail. Consequently, in order to improve the practicability of OCT technology in ophthalmology, effective data processing requires robust and accurate segmentation algorithms integrated into intelligent software solutions (see, FIGS. 6B, 6C). In addition, computer-aided detection and diagnosis based on automatic/semiautomatic robust algorithms will be essential in clinical studies where large datasets will be impractical for manual grading approaches.

Although cases studied were based on Stratus OCT images, the main purpose was to establish the feasibility of our quantitative methodology for OCT image analysis independent of the technology used. There is no doubt that if this methodology works well for Stratus OCT images, then it should perform better for FD-OCT images which have better resolution, and as such, ensure robust segmentation. To prove this, OCTRIMA segmentation was applied to raw images taken from FD-OCT devices. OCTRIMA segmentation results were obtained for an image obtained with the Bioptigen Spectral Domain Ophthalmic Imaging System (Bioptigen Inc, Research Triangle Park, N.C.). OCTRIMA segmentation results for an OCT image from a healthy eye obtained with a custom developed FD-OCT adapted from the anterior segment OCT system with ˜3 μm axial resolution. The system configuration has been detailed elsewhere (Q. Chen, et al. Invest Ophthalmol Vis Sci 2009; 0:iovs 09-4389v1-iovs. 09-4389; J. Wang, et al. Eye & Contact Lens;2: 44_{—}49 (2009)). Significant improvement has been obtained for the delineation of the INL, OPL and OS/RPE junction in the advanced OCT images analyzed.

Discussion

The most difficult part of biomedical image analysis is unsupervised segmentation, i.e., the automated localization and delineation of coherent structures of interest. However, the OCT denoising-enhancing method and quantitative methodology could be used to automatically segment the retinal structure in both normal and pathological subjects. Although the OCTRIMA software was initially developed for the quantitative analysis of Stratus OCT images, it is also potentially useful for the analysis of new OCT technologies, such as spectral domain and ultrahigh resolution based OCT technology, which provide images with higher resolution along with a dense map of the retina.

In order to accurately diagnose, study, and treat retinal diseases such as diabetic retinopathy, glaucoma and age-related macular degeneration (AMD), researchers have begun developing OCT quantitative tools. OCTRIMA helps ophthalmologists to explore Stratus OCT data by providing visualization and analysis methods, in conjunction with a segmentation method for extracting retinal layers and other structures, such as intraretinal and subretinal fluid-filled regions. The user interface allows further processing, so that users can diagnose and evaluate disease progression. In addition, OCTRIMA provides a manual correction tool that facilitates the interaction with the automated segmentation results, enabling significant improvements of segmentation accuracy.

The system herein, works well at isolating most retinal layers and structures in normal healthy controls and patients with early retinopathy and diabetic diffuse macular edema. Accordingly, a more robust and localized quantification of the retinal structure can be achieved using OCTRIMA software. The total time required to preprocess and automatically segment an OCT image (B-scan) is 20 seconds on a computer with INTEL® CORE™ 2 Extreme CPU Q9300 @ 2.53 GHz and 8 Gb. The inventors are currently improving the automated segmentation method to reduce the time needed to correct errors in segmentation. In the example above, a high degree of repeatability, reproducibility and reliability of the OCTRIMA software was obtained using data from ten control healthy eyes.

Conclusion:

The newly designed software package (OCTRIMA) is a robust and interactive computer aided retinal image analysis system for the assessment of retinal pathologies using Stratus OCT images. The user interaction with the software is mediated using a graphic user interface that offers various types of controls and menu options to facilitate a user-friendly environment to quantitatively assess the macula and the various cellular layers of the retina. The software implements a custom segmentation algorithm to accurately identify the boundaries of the intraretinal layers with minimized errors, which is functionally more efficient as compared to the commercial Stratus OCT software. Additionally, OCTRIMA offers a unique method to manually eliminate visible irregularities, if any, in the detected boundaries between the various cellular layers of the retina. This powerful capability ensures higher accuracy in the numerical data obtained from the measurements of the thickness and reflectance of each layer and the whole macula. Additional capabilities of the newly developed software include report generation for quantitative analysis of the macula and intraretinal layers per scan and per region.

OCTRIMA can also generate and display topographic maps for the thickness of each cellular layer of the retina, which provides a visual aid for better analysis of local structural changes, if any, in each ETDRS retinal region. Another potential advantage of OCTRIMA is the incorporation of a uniform method for reporting changes in thickness, which could be used as a framework for reporting treatment outcomes. Thus, OCTRIMA can be used to compare the efficacy of current and emerging therapies, as well as monitor the progression of disease in patients. Future work will include the adaptation of OCTRIMA into a more practical interface to handle the large quantities of measured raw data generated by the advanced OCT systems. In addition, the improvement of the compilation process to obtain high performance C++ source code without relying on external heavy-weight libraries will be also considered in the future development of OCTRIMA. The current implementation of the software is a single user based design for executing the application on the local machine. However, a networked multi-user design would be possible with a JAVA implementation of OCTRIMA, accessible world-wide using the internet. Moreover, OCTRIMA's retinal thickness measurements could be used as a primary or secondary end point for clinical trials of therapeutic agents for retinal tissue alterations under investigation by pharmaceutical companies. OCTRIMA will help physicians and their patients better evaluate the efficacy of therapeutic intervention.

### Example 11

### Evaluating the Effect of Speckle Denoising on the Estimation of Optical Properties of Intraretinal Layers Using Optical Coherence Tomography

Optical Coherence Tomography (OCT) is a rapidly emerging imaging technology that enables visualization of the cross sectional structure of the retina and anterior eye. OCT is usually employed for the measurement of retinal thickness. However, coherent reflected light carries more information characterizing the optical properties of tissue. Before the question of whether or not OCT can quantitatively measure the optical properties of retinal tissue can be answered affirmatively, better understanding and modeling of the OCT signal from the retinal structure is needed. The purpose of this study was to evaluate the effect of speckle denoising on the estimation of optical properties of the intraretinal layers on OCT image.

Methods: Speckle-free test Stratus OCT images from normal (168 scans) and pathological eyes (42 scans) were obtained after applying median filtering. Since the speckle pattern becomes additive white noise after the log-transformation, a Gaussian distribution approach of the speckles was considered. Therefore, the experiments were conducted on the OCT test images at different levels of Gaussian additive noise (5%, 10%, and 15%). Automatic/semi-automatic layer segmentation was performed using a custom-built algorithm (OCTRIMA). Specifically, the RNFL, GCL+IPL, INL, OPL, ONL, IS/OS and RPE layer were extracted with OCTRIMA.

Relative light-backscattering of two particular layers characterized by low (ONL) and highlight-backscatter (IS/OS) were used in the scattering coefficient (μₜ) analysis. These coefficients were calculated using a finite difference method assuming a single-scattering model. The μₜ was measured from the OCT signal by fitting a model relation to this signal from a ROI in an OCT image. To improve the numerical accuracy of the μₜ, the lateral coordinates of the blood vessel shadows were first extracted by using a blood vessel shadowgram technique. Then, these shadows were removed in each OCT image before calculating μₜ. The signal-to-mean square error ratio (S/MSE) and ratio measurements between the μₜ values obtained before and after noise removal were used in the quantitative analysis.

Results: AS/MSE improvement of 3%, 8% and 21% was obtained for 5% (P5F), 10% (P10F) and 15% (P15F) noise level, respectively (see Table 13). The μₜ decreased for the ONL and increased for the IS/OS when the noise level was augmented from 5 to 15% in OCT data from pathological subjects. A similar trend for the IS/OS was observed in healthy eyes (see Table 14). Once the Gaussian additive noise was removed, the μₜ of the ONL increased in both normal and pathological OCT data. On the contrary, the μₜ of the IS/OS decreased (see Table 14).

TABLE 13
S/MSE results obtained for 5%, 10% and 15% Gaussian
additive noise level before and after denoising.

| | | P5 | P5F | P10 | P10F | P15 | P15F |
|---|---|---|---|---|---|---|---|
| S/MSE | | (mm⁻¹) | (mm⁻¹) | (mm⁻¹) | (mm⁻¹) | (mm⁻¹) | (mm⁻¹) |
| Healthy | ONL | 1.64 | 1.70 | 1.57 | 1.69 | 1.40 | 1.67 |
| Eyes | IS/ | 1.99 | 2.06 | 1.92 | 2.05 | 1.77 | 2.05 |
| | OS | | | | | | |
| Patho- | ONL | 0.87 | 0.89 | 0.79 | 0.88 | 0.66 | 0.86 |
| logical | IS/ | 1.37 | 1.39 | 1.31 | 1.40 | 1.16 | 1.37 |
| Eyes | OS | | | | | | |

P5, P10 and P15 represents S/MSE values obtained when a 5%, 10% and 15% Gaussian additive noise was added to the OCT raw image after median filtering. In addition, P5F, P10F and P15F represents the values obtained after the speckle-free test OCT images corrupted with Gaussian additive noise were denoised. OCT data from pathological eyes were obtained from diabetic patients with early retinopathy.

TABLE 14
Scattering coefficient results obtained with the finite difference method.

| | | | | | | | | (P10F − | (P15F − |
|---|---|---|---|---|---|---|---|---|---|
| Scattering | | P5 | P5F | P10 | P10F | P15 | P15F | P5F)/P5F | P10F)/P10F |
| Coefficient | | (mm⁻¹) | (mm⁻¹) | (mm⁻¹) | (mm⁻¹) | (mm⁻¹) | (mm⁻¹) | (%) | (%) |
| Healthy | ONL | 2.02 ± 0.17 | 2.24 ± 0.20 | 2.10 ± 0.15 | 2.23 ± 0.21 | 2.05 ± 0.23 | 2.10 ± 0.22 | −0.08 | −6.00 |
| Eyes | IS/OS | 6.89 ± 0.36 | 6.94 ± 0.70 | 7.86 ± 0.32 | 6.88 ± 0.69 | 8.32 ± 0.59 | 7.08 ± 0.57 | −0.85 | 2.87 |
| Pathological | ONL | 2.77 ± 0.15 | 2.98 ± 0.16 | 2.62 ± 0.14 | 2.82 ± 0.13 | 2.51 ± 0.22 | 2.79 ± 0.29 | −5.39 | −1.15 |
| Eyes | IS/OS | 7.76 ± 0.93 | 7.22 ± 1.07 | 8.11 ± 0.82 | 7.15 ± 1.12 | 8.43 ± 0.48 | 7.41 ± 0.91 | −0.92 | 3.55 |

Discussion: Discrimination of different tissues based on differences in their scattering coefficient requires its accurate measurement from OCT data. In this study it was found that the scattering coefficients extracted from OCT images were more affected as the noise level increased. In addition, higher scattering coefficients were obtained for the pathological eyes independently of the noise level added to the OCT test images. The scattering coefficients obtained for the IS/OS were higher compared to the coefficients obtained for the low contrast layer(ONL).

It was also shown that it is possible to estimate the scattering coefficients of the intraretinal layers. Experiments are also being conducted to clearly show that the origin of the observed optical changes is the altered physiological state of the retina due to the progression of disease.

Conclusion: Localized quantitative measurement of the scattering coefficient of the intraretinal layers can provide additional information and may improve the clinical value of OCT by allowing quantitative discrimination between healthy and diseased cellular layers of the retina.

Future studies using phantom models will be conducted to determine the feasibility and full capability of this methodology to better explore the variability of optical properties of the retinal tissue.

### Example 12

### Comparing Quantitative Measurements Obtained Using Stratus OCT and an OCT Retinal Image Analysis (OCTRIMA) Software

The commercial time-domain Stratus OCT(Carl Zeiss Meditec, Dublin, Calif.) has a measurement capability limited to retinal thickness (RT) and cannot give quantitative information on intraretinal layers. In addition, Stratus OCT built-in algorithms identify the outer retinal boundary as the inner border of the RPE layer rather than identifying its outer border, which, probably corresponds to the true anatomic allocation of the outer boundary.

In an effort to provide additional retinal quantifications along with accurate automatic/semi-automatic detection, a software tool was developed for OCT retinal image analysis (OCTRIMA) which is an interactive, user-friendly stand-alone application for analyzing OCT retinal images. OCTRIMA calculates the RT as the distance between the ILM and the anterior boundary of the red reflective layer corresponding to the RPE-choriocapillaris complex. In this study, the volume and RT were compared and measured by both, OCTRIMA and Stratus OCT.

Methods: Standard macular mapping by Stratus OCT were performed in ten healthy eyes from 5 normal subjects, ranging in age from 25 to 34 years (mean age 29 years). OCTRIMA's thickness measurements were obtained by calculating the thickness between ILM and RPE inner boundary whereas automated Stratus OCT results are currently calculated between ILM and ONL (outer boundary). Stratus OCT retinal thickness measurements were compared with the OCTRIMA's measurements in each of the nine ETDRS macular regions. Differences in the measurement of the macular volume were also calculated. Moreover, the OCTRIMA and Stratus OCT thickness calculations' agreement with measurements obtained with frequency-domain OCT systems was also evaluated.

Results: Table 15 shows the comparison between Stratus OCT retinal thickness measurements and the OCTRIMA measurements obtained using the true anatomic location of the outer retinal boundary (i.e. RPE's inner boundary). Differences in the measurement of the total macular volume are also included and are expressed in cubic millimeters (mm³)

| | | | Mean | Percent of the |
|---|---|---|---|---|
| | Stratus | | Absolute | STRATUS OCT |
| Retina Macular | OCT | OCTRIMA | Difference | measurement |
| Regions | (μm) | (μm) | (μm) | (%) |
| Fovea | 184.50 | 215.09 | 30.59 | 16.58 |
| Superior Inner | 283.90 | 311.50 | 27.60 | 9.72 |
| Temporal Inner | 281.00 | 313.26 | 32.26 | 11.48 |
| Inferior Inner | 280.70 | 304.13 | 23.43 | 8.35 |
| Nasal Inner | 266.20 | 298.20 | 32.00 | 12.02 |
| Superior Outer | 243.30 | 272.49 | 29.19 | 12.00 |
| Temporal | 259.00 | 281.49 | 22.49 | 8.68 |
| Outer | | | | |
| Inferior Outer | 232.30 | 250.71 | 18.41 | 7.92 |
| Nasal Outer | 224.40 | 248.80 | 24.40 | 10.87 |
| Mean | 250.59 | 277.30 | 26.71 | 10.85 |
| Range | (185-284) | (215-313) | (18-32) | (8-17) |

Total macular volume (mm³)

| | | | |
|---|---|---|---|
| 6.99 | 7.68 | 0.69 | 9.93 |

Note that the mean difference for the foveal center (R1) included only 17% of the measured value obtained by the commercial Stratus system. The mean difference results for R2-R9 included 8-12% of the Stratus OCT measurements. Moreover, the mean difference between Stratus OCT retinal thickness measurements and OCTRIMA thickness measurements was 27 microns (range, 18-32 microns), or 11% of the measured Stratus OCT retinal thickness. Total macular volume, a measure derived from thickness in all datapoint of the macula, was 10% higher by OCTRIMA compared to Stratus OCT results, also supporting an average difference of 10% in thickness measurements.

Discussion: After comparing Stratus OCT retinal thickness measurements with the measurements obtained using the true anatomic location of the outer retinal boundary (i.e. RPE's inner boundary), the mean thickness difference was 27 μm (ranged, 18-32 μm see Table 15). Similar results have been recently reported (mean=35.5 μm, range 27-45 μm) (Sadda et al., IOVS, 2007; 48: 839-848). Similar thickness differences have been found between Stratus OCT and FD-OCT systems, which use the same method of the OCTRIMA software to calculate the total retinal thickness.

This particular observation enhances the reliability of OCTRIMA software when compared to FD-OCT systems. Although, mean difference values per ETDRS region included only 8-17% of the measured total values obtained by the commercial Stratus OCT system, further investigations related to the reliability of the segmentation of the anatomically correct location of the RPE are required before clinicians can fully rely on this newly defined true retinal thickness.

Conclusions: These results are comparable to those obtained using frequency-domain OCT systems which clearly demonstrates the reliability of OCTRIMA software. Hence, OCTRIMA is a good candidate for potential OCT based studies examining morphologic characteristics of changes in retinal structure.

### Example 13

### Different Trends Observed for Age-related Changes of the Macula Affecting the Ganglion Cells and Retinal Pigment Epithelium

Optical coherence tomography (OCT) is a noninvasive, noncontact diagnostic tool that can provide in vivo, real-timecross-sectional imaging of the retina. The commercial time-domain Stratus OCT (Carl Zeiss Meditec, Dublin, Calif.) provides images with 8-10-μm axial resolution and a maximum of 512 transverse×1024 axial datapoints per image acquired in 1.25 seconds. Cross-sectional OCT images of the retina have been found to correlate well with retinal histology.

In an effort to obtain quantitative data of intraretinal structures a software tool was developed for Stratus OCT retinal image analysis (OCTRIMA). OCTRIMA is able to minimize segmentation errors and give quantitative information of intraretinal structures. The software can distinguish 7 layers of the retina on OCT images based on their optical densities: the retinal nerve fiber layer (RNFL), the ganglion cell+inner plexiform layer complex (GCL+IPL), the inner nuclear layer (INL), the outer plexiform layer (OPL), the outer nuclear layer (ONL), the inner-outer segment border (IS/OS) and the retinal pigment epithelial layer (RPE).

The quantification of changes in macular thickness as well as thickness of each cellular layer of the retina is important as it provides useful information for detecting pathologic changes and diagnosing retinal diseases. However, proper diagnosis of ophthalmic diseases requires a substantive knowledge of the expected thickness of the macula and its layers. The retina undergoes several structural changes with aging. One of these processes is the thinning of the retina and retinal nerve fiber layer (RNFL) as described in histological studies. With the introduction of newer imaging methods, several studies have shown the thinning of peripapillary RNFL with age using OCT and there have been some studies observing total retinal thickness decrease. The cause of age-related nerve fiber loss is thought to be the apoptosis of retinal ganglion cells. The purpose of the present study was to describe age-related changes of the human macula in vivo using optical coherence tomography(OCT).

Methods: A total of 29 right and 26 left eyes of 55 healthy volunteers were recruited in this prospective study. The age of the volunteers ranged from 21 to 88 years with a median of 42 years (mean age 45, 25 years). Thirty-nine women (71%) and 16 men (29%) participated. 14 subjects underwent uneventful phaco emulsification surgery with PCL implantation 6-12 months prior to enrollment (age range 72-88 years).

The inclusion criteria for all participants were: best-corrected Snellen visual acuity of 20/20, preoperative spherical and cylindrical correction within ±3.0 diopters (D). Exclusion criteria were treated glaucoma, any abnormalities of the disc or the retinal nerve fiber layer, particularly glaucoma-like cupping; family history of glaucoma or any other hereditary eye disease, IOP≧20 Hgmm in the history, any retinal disease, even AMD st.1; diabetes mellitus or other systemic disease possibly affecting the eye except controlled hypertension; history of intraocular surgery (or any kind of laser therapy including refractive surgery) in 6 months time before the examination. In patients in whom both eyes were eligible, the study eye was selected randomly.

All subjects underwent routine ophthalmic examination including best corrected visual acuity, assessment of intraocular pressure(IOP), slit lamp biomicroscopy and binocular ophthalmoscopy after pupil dilatation.

Optical coherence tomography scans were performed using Stratus OCT (Carl Zeiss Meditec, Inc., Dublin, Calif., USA). Before recording the OCT images each eye of each subject was dilated with tropicamide 0.5%. All scans used an internal fixation target and were performed by the same operator. Standard macular mapping with “Macular Thickness Map” protocol was used for all imaging in the study which consists of six evenly spaced radial line centered on the fovea, each having a 6 mm transverse length. Average thickness was extracted for all retinal layers.

Sigmoid and linear functions were fitted using the least squares regression technique to decide which model describes better the kinetics of retina layer thickness changes occurring with age. The models were compared using the residual standard errors (RSE), the Akaike Information Criterion (AIC) and the F test on residual sum of squares.

Results: A decrease in thickness of RNFL was observed with age which was better described by a sigmoid model, however F-test was not significant. In the case of GCL+IPL a sigmoidal trend gave a significantly better fit for the observed thickness decrease with age. The thickness of the RPE layer showed a linear increase related to age, as only a linear model could be fitted (linear correlation r=0.36, p=0.006). All other layers did not show any significant age-related thickness changes (Table 16).

TABLE 16
Statistical results for the age-related trends
in the various retinal layers of the macula.

| | Linear model | | Sigmoid model | | | |
|---|---|---|---|---|---|---|
| | RSE | AIC | RSE | AIC | F-test | p |
| RNFL | 2.556 | 265.26 | 2.508 | 261.05 | 2.03 | 0.14 |
| GCL + IPL | 4.968 | 338.37 | 4.703 | 330.24 | 4.06 | 0.023 |
| INL | No correlation | | | | | |
| OPL | | | | | | |
| ONL | | | | | | |
| RPE | Linear correlation | | | | | |
| Total Retina | 9.9 | 410.11 | 9.8 | 411.58 | — | |

RSE: residual standard errors,
AIC: Akaike Information Criterion.

Conclusions: These results indicate that age-related cellular changes of the macula may occur with different kinetics. The known decrease in the ganglion cell number (and thus GCL+IPL thickness) follows a sigmoidal trend, which is paralleled by a decrease in the nerve fiber layer following the same, although less evident trend. The data herein, provide proof that physiological apoptosis occurs in the retina with age.

The linear thickening of the RPE might be in correlation with described age-related histological changes of the Bruch membrane. This study demonstrates the different kinetics in the ageing retina observed in vivo with the help the custom-built OCTRIMA software.

Optical Coherence Tomography for Characterization of Ocular Disease Resulting from Neurodegenerative Disorder.

Although the various embodiments have been described above with respect to diabetic retinopathy, the invention is not limited in this regard. Rather the OCT data collection and analysis methods described above can be adapted for purposes of assessing a level of ocular diseases or disorders resulting from neurodegenerative disorder, including, but not limited to ocular diseases or disorders resulting from multiple sclerosis, Parkinson's disease, and Alzheimer's disease.

In Vivo Evaluation of Retinal Neurodegeneration in Patients with Multiple Sclerosis

Multiple sclerosis (MS) is a chronic inflammatory disorder that affects the central nervous system. The disease is characterized by demyelination that leads to axonal dysfunction and neuronal loss. Unmyelinated neuronal axons offer a good possibility to examine axonal loss as the thickness of the myelin sheath does not affect the nerve thickness results. The innermost layer of the retina is the retinal nerve fiber layer (RNFL) being comprised of the axons of the retinal ganglion cells which get myelin sheath only after leaving the eye through the lamina cribrosa. Therefore, the thickness measurement of the RNFL might be a good marker of the axonal damage in MS patients. Optical coherence tomography (OCT) is a non-invasive, non-contact diagnostic tool that provides high-resolution cross-sectional images of the retina This technique enables, among others, the measurement of the thickness of the RNFL around the optic disc and also the thickness and volume of the macula lutea in vivo. With the use of OCT image processing, not only the thickness of the total retina but also the thickness of the intraretinal layers can be measured in the macular area.

The loss of retinal nerve fibers around the optic disc (circumpapillary RNFL—cpRNFL) has been found in the eyes of MS patients both with and without optic neuritis (ON) in the history. However, recent studies have shown that also macular thickness and volume are decreased in the eyes of patients with MS, presumably caused by the thinning of the ganglion cell layer (GCL) and inner plexiform layer (IPL).

The purpose of our study was to assess macular morphology in patients with MS with or without ON in previous history and also to determine which OCT parameter has the greatest ability to detect neuronal damage in patients with MS. We could demonstrate that the thickness of the ganglion cell complex (GCC) is the most sensitive marker for the detection of neuronal loss due to ON and we could also show that there are signs of axonal degeneration even without ON in previous history. The thickness of the GCL+IPL complex and the GCC in the macula showed the strongest correlation with the clinical measure of disability measured by the EDSS score.

Methods: Thirty-nine patients with relapsing-remitting multiple sclerosis meeting the revised McDonald criteria were consecutively recruited from the Department of Neurology of Semmelweis University between October 2008 and June 2011 in this cross-sectional case-control study. Exclusion criteria for all patients were: (1) spherical or cylindrical correction higher than 3.0 diopters, (2) the presence of any retinal disease or optic neuropathy including glaucoma, except ON, (3) intraocular pressure higher than 20 mmHg in the medical history, (4) previous eye surgery, (5) amblyopia, (6) last ON episode less than 6 months prior to enrollment, (7) bad fixation cooperation during the OCT examination (e.g. due to nystagmus) and (8) low signal strength of the OCT images (SS:6). Five eyes were excluded from the study due to the presence of retinal disease (1 eye), acute ON (1 eye), low signal strength of the OCT image due to media opacity (1 eye) and amblyopia (2 eyes). Thirty-three randomly selected eyes of thirty-three age-matched controls were also examined with OCT. The eligibility criteria for control subjects were best-corrected Snellen visual acuity of 20/20 and the lack of any ocular or systemic diseases. Demographic and clinical characteristics including age, gender and duration of disease are listed in Table 17.

TABLE 17
Clinical characteristics of the study patients.

| | Control | MS |
|---|---|---|
| Characteristic | (n = 33) | (n = 39) |
| Age, mean ± SD, y | 34.3 ± 8.3 | 34.0 ± 8.2 |
| Age, median (range) | 33 (21-52) | 34 (19-53) |
| No. (%) female | 23 (69.7) | 27 (69.2) |
| ON-affected eye, No. (%) | NA | 39 (53.0) |
| Disease duration, mean ± SD, y | NA | 6.5 ± 3.9 |

NA: not applicable,
MS: multiple sclerosis,
SD: standard deviation

All participants were treated in accordance with the tenets of the Declaration of Helsinki. Institutional Review Board approval was obtained for all study protocols (Semmelweis University Regional and Institutional Committee of Sciences and Research Ethics). Written informed consent was obtained from all participants in this study.

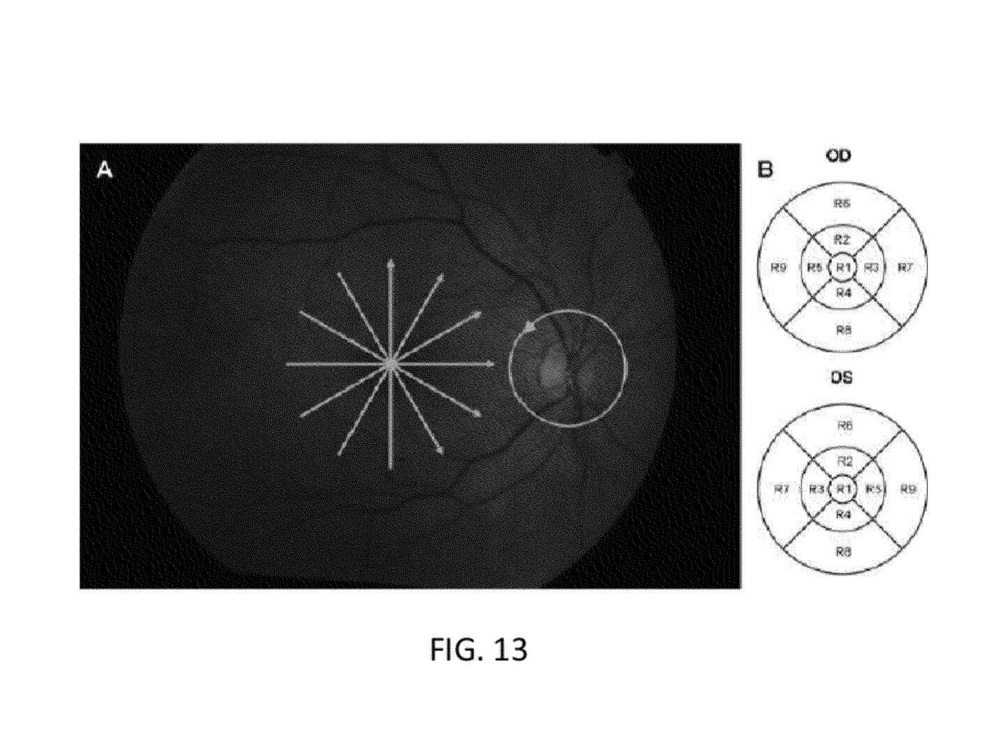

All patients underwent an ophthalmic examination including best-corrected Snellen visual acuity, assessment of intraocular pressure, slit lamp biomicroscopy and binocular ophthalmoscopy with pupil dilation. The same day, OCT examination was performed on each eye using a Stratus OCT device (Carl Zeiss Meditec, Dublin, Calif., USA). “Fast RNFL map protocol” consisting of three circular scans with diameters of 3.4 mm centered on the optic disc was performed (FIG. 13). The mean overall and sectoral (superior, nasal, inferior and temporal) cpRNFL thickness values were recorded for each eye. The mean overall cpRNFL thickness was calculated by averaging the thickness values in the four sectors. To assess the thickness of the intraretinal layers of the macula, each eye was scanned using the “Macular thickness map” protocol consisting of six evenly spaced radial lines centered on the fovea, each having a 6 mm transverse length (FIG. 13).

FIG. 13 shows the retinal scanning used in the study. The arrows in the macula and the circle around the optic nerve head show the locations of the OCT scans made. (B) The distribution of ETDRS regions for the right (OD) and left eye (OS).

The patients underwent a comprehensive neurological examination within one week of the ophthalmic examination. To assess physical disability, the Expanded Disability Status Scale (EDSS) score was determined for each patient.

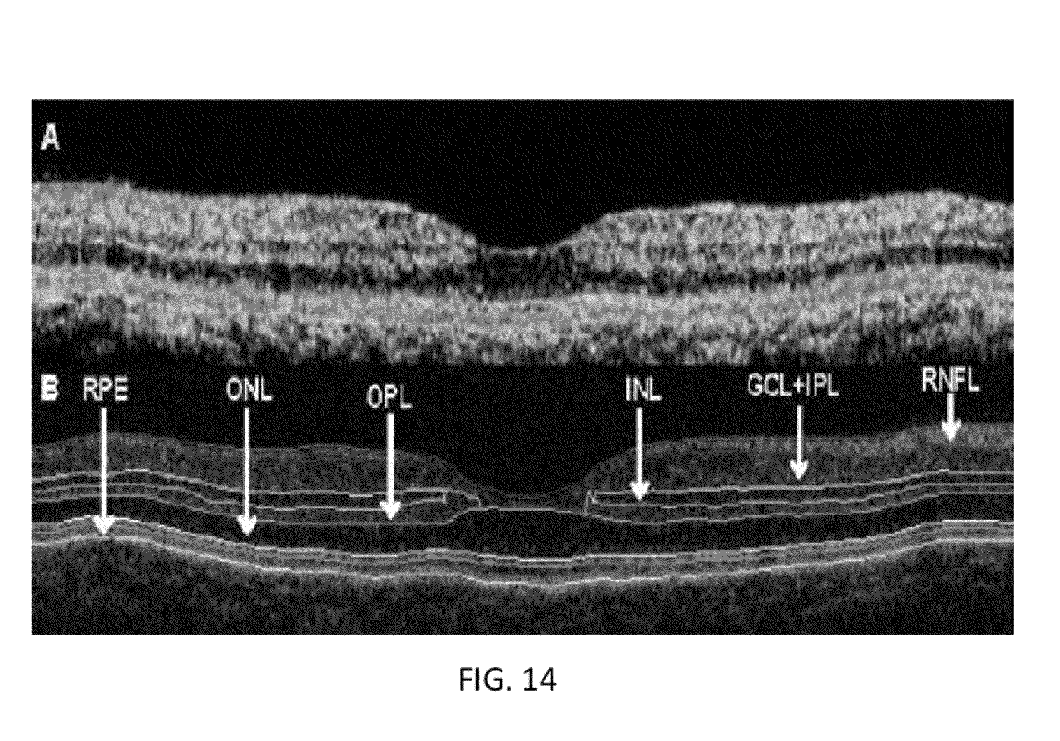

The raw macular OCT data were exported from the Stratus OCT device and further processed using a computer-aided grading methodology for OCT retinal image analysis (OCTRIMA). The OCTRIMA software integrates a novel denoising and edge enhancement technique along with a segmentation algorithm. Moreover, the software gives quantitative information of intraretinal structures and facilitates the analysis of other retinal features that may be of diagnostic and prognostic value, such as the morphology and reflectivity by enabling the segmentation of the various cellular layers of the retina. The OCTRIMA software enables the segmentation of 6 layers of the retina on OCT images based on their optical densities: the RNFL, the GCL+IPL complex, the inner nuclear layer (INL), the outer plexiform layer (OPL), the outer nuclear layer (ONL) and retinal pigment epithelium (RPE). We have previously shown the high repeatability and reproducibility of OCTRIMA measurements in normal subjects with undisrupted retinal structure similarly to what is observed in patients with MS. The reproducibility was the highest for the thickness measurements of the ONL, GCC, GCL+IPL and RNFL, the inter- and intraexaminer, intervisit variabilities <<6 μm for all layers and all comparisons) being under the resolution of the Stratus OCT device for all layers. The OCT scan of a healthy macula before and after segmentation with OCTRIMA can be seen on FIG. 14.

FIG. 14 shows macular image segmentation using OCTRIMA. A is the image of a healthy macula scanned by Stratus OCT. B is the same OCT scan processed with OCTRIMA. We note the ONL segment includes the IS of photoreceptors and the external limiting membrane, which are not resolved by the Stratus OCT device. Abbreviations: GCL+IPL, ganglion cell layer and inner plexiform layer complex; INL, inner nuclear layer; ONL, outer nuclear layer; OPL, outer plexiform layer; RNFL, retinal nerve fiber layer; RPE, retinal pigment epithelium.

It is important to note that OCTRIMA measures the thickness of the total retina between the inner limiting membrane and the inner boundary of the photoreceptor outer segment/RPE junction. On the contrary, Stratus OCT measures the thickness of the total retina between the inner limiting membrane and the photoreceptor inner segment-outer segment junction. We also note that the ONL is actually enclosing the external limiting membrane and the inner segment of the photoreceptors but in the standard 10!m resolution OCT image this thin membrane can not be visualized clearly making the segmentation of the inner segment difficult. Thus this layer classification is our assumption and does not reflect the actual anatomic structure.

The thickness of the total retina and the intraretinal layers were measured in the nine macular regions defined by the Early Treatment Diabetic Retinopathy Study (ETDRS) (FIG. 13). Since the number of sampling points is different at the central (R1), pericentral (R2-R5) and peripheral (R6-R9) regions because of the radial spoke pattern used in the scanning protocol of Stratus OCT, a weighted mean thickness (WMT) was calculated instead of averaging retinal thickness results in the 9 macular regions. The WMT was generated using the following equation:

$$WMT = \frac{R 1}{36} + \frac{R 2 + R 3 + R 4 + R 5}{18} + \frac{( R 6 + R 7 + R 8 + R 9 ) \times 3}{16}$$

The WMT values for the RNFL, GCL+IPL, GCC, INL, OPL, ONL, RPE and the total retina were obtained for each eye. Since the GCC is composed by the RNFL and GCL+IPL consisting of distal and proximal parts of the retinal ganglion cells, the thickness of the GCC was used to describe the integrity of the ganglion cells.

The correlation between the disease duration, age, EDSS score and the cpRNFL and macular intraretinal thickness parameters was calculated by linear correlation. Furthermore, the correlation between the cpRNFL parameters and the thickness of the intraretinal structures was also determined by linear correlation.

The eyes of the MS patients were divided into two study groups for further analyses. The first group was composed of 39 eyes which had ON at least 6 months prior to enrollment. All eyes in this group had only one previous episode of ON. The second group was composed of 34 eyes which had no history of ON. The diagnosis of optic neuritis was based on the patient's medical history and defined by clinical symptoms such as decreased visual acuity developing in few days, pain on eye movement, abnormal response on visual evoked potential examination confirming prechiasmal lesion and decrease in the critical flicker frequency (CFF). CFF is a routine examination for the assessment of optic nerve function. Particularly, in the context of optic neuritis, the CFF is known to be decreased in the acute phase of ON and also it can show impairment after the recovery. Therefore, measuring the CFF can help to establish the diagnosis.

All measured thickness values were compared among the groups using mixed model ANOVA. Receiver operating characteristic (ROC) curves were constructed to describe the ability of each parameter to discriminate between the eyes of MS patients not affected with ON and the eyes of the control group.

Statistical analyses were performed using Statistica 8.0 (Statsoft Inc., Tulsa, Okla., USA) and SPSS15.0 (SPSS Inc., Chicago, Ill., USA). A p value of <0.05 was considered statistically significant.

Results All eyes had a Snellen visual acuity of 1.0. The strongest correlation was observed between the EDSS and the GCL+IPL, GCC and mean overall cpRNFL (p=0.007, p=0.007 and p=0.008, respectively; r=−0.43 for all variables) while the correlations with the inferior cpRNFL, superior cpRNFL and the WMT of the RNFL in the macula was weaker (p=0.02, p=0.05 and p=0.05, respectively; r=−0.38, r=−0.33 and r=−0.32, respectively). The remaining intraretinal layers and cpRNFL parameters showed no correlation with the EDSS. There was no correlation between any of the thickness values measured and either disease duration or age.

The mean overall cpRNFL thickness and the cpRNFL thickness in the superior, nasal, inferior and temporal quadrants was significantly decreased in the eyes of MS patients previously affected with ON compared to the non-affected eyes of MS patients (see Table 18). Each cpRNFL thickness parameter was significantly lower in the ON-affected eyes compared to controls except for the cpRNFL thickness in the nasal quadrant. However, the eyes not affected with ON showed significantly lower cpRNFL thickness values compared to the control group only in the temporal quadrant. The cpRNFL was thinner in the superior, nasal, inferior and temporal quadrants by 16%, 11%, 16% and 27%, respectively in the ON-affected eyes compared to controls and by 6%, 4%, 4% and 17%, respectively in the eyes without ON in medical history compared to controls.

TABLE 18
WMT values of the intraretinal layers and the total 495 retina in the study groups.

| | Thickness, μm, mean ± SD | | | p values | | |
|---|---|---|---|---|---|---|
| | Control | NE group | AE group | NE vs. | AE vs. | NE vs. |
| | (33 eyes) | (34 eyes) | (39 eyes) | Control | Control | AE |
| Intraretinal layer | | | | | | |
| RNFL | 38 ± 3 | 34 ± 3 | 31 ± 5 | 0.001* | <0.001* | <0.001* |
| GCL + IPL | 72 ± 4 | 63 ± 6 | 54 ± 7 | <0.001* | <0.001* | <0.001* |
| GCC | 109 ± 6 | 98 ± 8 | 85 ± 12 | <0.001* | <0.001* | <0.001* |
| INL | 33 ± 2 | 34 ± 2 | 33 ± 2 | 0.541 | 0.740 | 0.622 |
| OPL | 32 ± 2 | 32 ± 2 | 32 ± 2 | 0.612 | 0.721 | 0.210 |
| ONL | 79 ± 5 | 81 ± 7 | 81 ± 6 | 0.196 | 0.087 | 0.396 |
| RPE | 12 ± 1 | 12 ± 1 | 12 ± 1 | 0.950 | 0.589 | 0.390 |
| Total Retina | 290 ± 8 | 283 ± 11 | 268 ± 14 | 0.013* | <0.001* | <0.001* |
| cpRNFL sector | | | | | | |
| Mean Overall | 103 ± 7 | 94 ± 19 | 85 ± 14 | 0.101 | <0.001* | 0.037* |
| Superior | 131 ± 10 | 122 ± 21 | 110 ± 19 | 0.051 | <0.001* | 0.001* |
| Nasal | 77 ± 13 | 81 ± 21 | 69 ± 17 | 0.328 | 0.066 | 0.002* |
| Inferior | 130 ± 16 | 124 ± 18 | 109 ± 21 | 0.141 | <0.001* | <0.001* |
| Temporal | 73 ± 13 | 61 ± 12 | 53 ± 13 | 0.001* | <0.001* | 0.002* |

(*statistically significant)
SD: standard deviation;
NE: non-affected eye;
AE: affected eye;
RNFL: retinal nerve fiber layer;
GCL + IPL: ganglion cell layer and inner plexiform layer complex;
GCC: ganglion cell complex;
INL: inner nuclear layer;
OPL: outer plexiform layer;
ONL: outer nuclear layer;
RPE: retinal pigment epithelium.

The WMT of the total retina, RNFL, GCL+IPL and GCC showed a significant decrease in both the ON-affected and non-affected eyes of MS patients compared to the control group (Table 18). Furthermore, the eyes previously affected with ON had significantly lower thickness values in these layers than the eyes not affected with ON. There was no statistically significant difference in any of the remaining intraretinal layers among the groups.

The strongest correlation was observed between the mean overall cpRNFL thickness and the thickness of the GCL+IPL and GCC in the macula (r=0.76 and r=0.75, respectively) while the correlation was weaker in the case of the total retina in the macula (r=0.68). An average 10 μm loss of the average cpRNFL thickness was associated with 7.5 μm reduction in the total retinal thickness and also a 7.5 μm reduction in the thickness of the GCC, the latter resulting from a 5.3 μm reduction of the GCL+IPL and a 2.2 μm reduction of the RNFL.

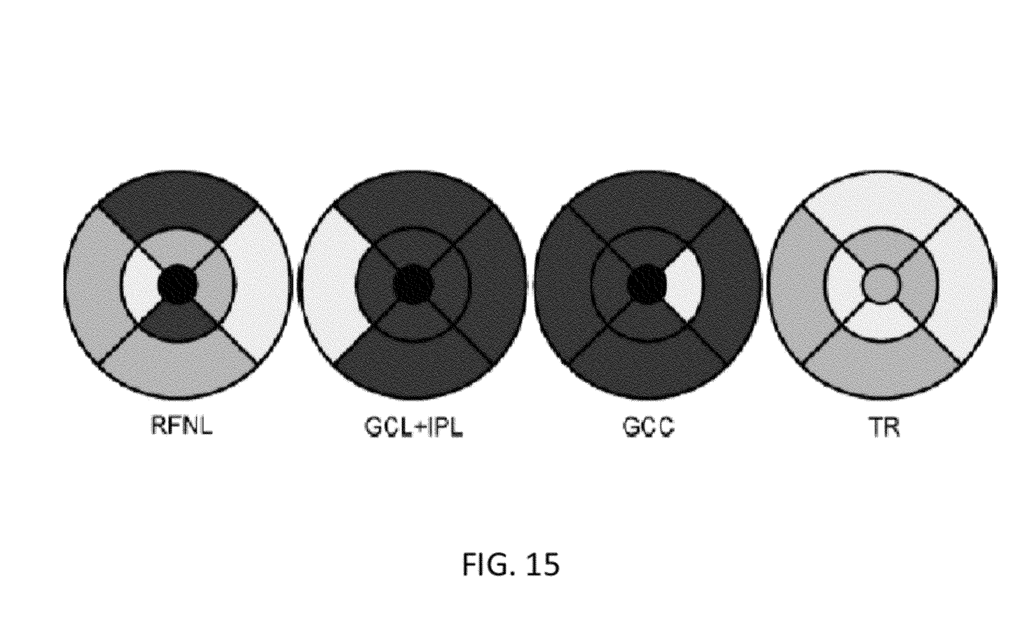

The thickness of the RNFL in the macula was significantly decreased in the non-affected eyes of MS patients compared to the healthy eyes in the inner inferior (R4), inner temporal (R5), outer superior (R6) and outer nasal (R7) regions (see Table 19 and FIG. 15).

FIG. 15. Regional differences between the non-affected eyes of MS patients and healthy eyes. The colors show the extent of thinning based on the p-values of the thickness comparisons. All representative numerical data are in Table 19. The color codes are as follows: dark grey: p<0.001, white: 0.001<p<0.05, light grey: p>0.05. We note the central subfield (R1: black color) was excluded from the analysis for the layers which are not present in the foveal area. Abbreviations: GCC, ganglion cell complex; GCL+IPL, ganglion cell layer and inner plexiform layer complex; RNFL, retinal nerve fiber layer; TR, total retina.

In the eyes affected with ON, macular RNFL thickness was significantly lower in each region compared to the control group and it was also lower compared to the eyes not affected with ON in each region except for the outer temporal region (R9). The thinning of the GCL+IPL was observed in each ETDRS region in the non-affected eyes of MS patients compared to controls and in the ON-affected eyes of MS patients compared to both healthy and non-affected eyes. The thickness of the GCC was found to be significantly lower in each region in the non-affected eyes of MS patients compared to the control group and in the ON-affected eyes compared to both the control group and the non-affected eyes (Table 19 and FIG. 15). The thickness of the total retina was significantly thinner in each region except for the central region (R1) in the affected eyes of the MS patients compared to the healthy eyes while it was thinner in each region in the affected eyes compared to the non-affected. For the comparison of the eyes of MS patients not affected with ON versus healthy subjects' eyes there was a significant thinning only in the inner inferior (R4), inner temporal (R5), outer superior (R6) and outer nasal (R7) regions (see Table 19 and FIG. 15).

TABLE 19
Regional differences in the thickness of the intraretinal layers 502
in the groups which showed statistically significant difference.

| | Thickness, μm, mean ± SD | | | p values | | |
|---|---|---|---|---|---|---|
| | Control | NE group | AE group | NE vs. | AE vs. | NE vs. |
| | (33 eyes) | (34 eyes) | (39 eyes) | Control | Control | AE |
| RNFL | | | | | | |
| R1 | NL | NL | NL | NA | NA | NA |
| R2 | 27 ± 4 | 26 ± 4 | 23 ± 5 | 0.172 | <0.001* | 0.002* |
| R3 | 24 ± 4 | 23 ± 4 | 19 ± 6 | 0.291 | <0.001* | <0.001* |
| R4 | 30 ± 4 | 25 ± 4 | 21 ± 6 | <0.001* | <0.001* | 0.001* |
| R5 | 19 ± 5 | 15 ± 4 | 13 ± 5 | 0.001* | <0.001* | 0.027* |
| R6 | 51 ± 5 | 46 ± 4 | 41 ± 5 | <0.001* | <0.001* | <0.001* |
| R7 | 53 ± 6 | 47 ± 4 | 42 ± 7 | 0.001* | <0.001* | <0.001* |
| R8 | 40 ± 3 | 38 ± 2 | 35 ± 7 | 0.107 | <0.001* | 0.001* |
| R9 | 28 ± 4 | 25 ± 4 | 23 ± 6 | 0.051 | <0.001* | 0.091 |
| GCL + | | | | | | |
| IPL | | | | | | |
| R1 | NL | NL | NL | | | |
| R2 | 96 ± 6 | 87 ± 9 | 72 ± 11 | <0.001* | <0.001* | <0.001* |
| R3 | 95 ± 6 | 87 ± 11 | 71 ± 10 | <0.001* | <0.001* | <0.001* |
| R4 | 94 ± 5 | 84 ± 8 | 72 ± 9 | <0.001* | <0.001* | <0.001* |
| R5 | 95 ± 5 | 85 ± 8 | 73 ± 10 | <0.001* | <0.001* | <0.001* |
| R6 | 67 ± 5 | 57 ± 8 | 49 ± 9 | <0.001* | <0.001* | <0.001* |
| R7 | 72 ± 6 | 61 ± 9 | 52 ± 10 | <0.001* | <0.001* | <0.001* |
| R8 | 61 ± 5 | 54 ± 5 | 47 ± 7 | <0.001* | <0.001* | <0.001* |
| R9 | 69 ± 6 | 63 ± 6 | 54 ± 7 | 0.001* | <0.001* | <0.001* |
| GCC | | | | | | |
| R1 | NL | NL | NL | | | |
| R2 | 124 ± 8 | 112 ± 11 | 95 ± 14 | <0.001* | <0.001* | <0.001* |
| R3 | 120 ± 8 | 109 ± 12 | 90 ± 15 | 0.001* | <0.001* | <0.001* |
| R4 | 124 ± 6 | 109 ± 11 | 93 ± 14 | <0.001* | <0.001* | <0.001* |
| R5 | 113 ± 6 | 100 ± 11 | 86 ± 13 | <0.001* | <0.001* | <0.001* |
| R6 | 117 ± 8 | 103 ± 9 | 91 ± 13 | <0.001* | <0.001* | <0.001* |
| R7 | 125 ± 8 | 109 ± 10 | 93 ± 15 | <0.001* | <0.001* | <0.001* |
| R8 | 101 ± 6 | 92 ± 7 | 82 ± 11 | <0.001* | <0.001* | <0.001* |
| R9 | 97 ± 7 | 89 ± 7 | 78 ± 10 | <0.001* | <0.001* | <0.001* |
| Total | | | | | | |
| retina | | | | | | |
| R1 | 234 ± 17 | 237 ± 20 | 226 ± 14 | 0.773 | 0.171 | <0.001* |
| R2 | 324 ± 13 | 319 ± 12 | 299 ± 16 | 0.115 | <0.001* | <0.001* |
| R3 | 322 ± 13 | 318 ± 14 | 298 ± 17 | 0.231 | <0.001* | <0.001* |
| R4 | 322 ± 11 | 312 ± 10 | 295 ± 15 | 0.001* | <0.001* | <0.001* |
| R5 | 312 ± 12 | 302 ± 11 | 288 ± 14 | 0.002* | <0.001* | <0.001* |
| R6 | 293 ± 8 | 283 ± 13 | 268 ± 16 | 0.006* | <0.001* | <0.001* |
| R7 | 301 ± 10 | 290 ± 14 | 272 ± 18 | 0.006* | <0.001* | <0.001* |
| R8 | 271 ± 7 | 267 ± 12 | 254 ± 14 | 0.103 | <0.001* | <0.001* |
| R9 | 271 ± 10 | 265 ± 13 | 253 ± 12 | 0.080 | <0.001* | <0.001* |

For the distribution of the ETDRS regions see FIG. 13.
(*statistically significant.)
SD: standard deviation;
NE: non-affected eye;
AE: affected eye;
NL: no layer;
NA: not applicable;
RNFL: retinal nerve fiber layer;
GCL + IPL: ganglion cell layer and inner plexiform layer complex;
GCC: ganglion cell complex;
INL: inner nuclear layer;
OPL: outer plexiform layer;
ONL: outer nuclear layer;
RPE: 509 retinal pigment epithelium.

The largest area under the curve (AUC) value for the discrimination between the non-affected eyes of MS patients and the eyes of the control group was obtained for the WMT of the GCC (0.892) (see Table 20). The AUC values for the thickness data obtained by the built-in software of the Stratus OCT—namely the cpRNFL thickness in the temporal quadrant and the thickness of the total retina—were below that of the GCC, 0.745 and 0.709, respectively. Within the GCC, the largest AUC was obtained for the inner temporal and outer nasal regions (R5 and R7, respectively) (0.867 and 0.851) for the discrimination between non-affected eyes of MS patients and controls (see Table 20).

TABLE 20
Results of the ROC analysis for the WMT thickness 512 variables
which showed significant difference between the eyes not affected
with ON and controls and for the regional GCC thickness values.

| | | Asymptotic 95% CI | | Cutoff point | | |
|---|---|---|---|---|---|---|
| Variable | AUC | Lower bound | Upper bound | (μm) | Sensitivity | Specificity |

WMT values of the intraretinal layers

| | | | | | | |
|---|---|---|---|---|---|---|
| GCC | 0.892 | 0.818 | 0.966 | 104 | 76% | 74% |
| GCL + IPL | 0.869 | 0.785 | 0.952 | 68 | 79% | 77% |
| RNFL | 0.809 | 0.707 | 0.911 | 35 | 82% | 56% |
| Temporal cpRNFL | 0.745 | 0.628 | 0.861 | 67 | 73% | 68% |
| Total Retina | 0.709 | 0.585 | 0.834 | 288 | 73% | 62% |

Regional thickness values for the GCC

| | | | | | | |
|---|---|---|---|---|---|---|
| R2 (inner superior) | 0.792 | 0.683 | 0.902 | 91 | 85% | 62% |
| R3 (inner nasal) | 0.740 | 0.616 | 0.863 | 91 | 76% | 65% |
| R4 (inner inferior) | 0.843 | 0.751 | 0.938 | 91 | 79% | 77% |
| R5 (inner temporal) | 0.867 | 0.778 | 0.957 | 90 | 82% | 74% |
| R6 (outer superior) | 0.839 | 0.747 | 0.931 | 63 | 76% | 74% |
| R7 (outer nasal) | 0.851 | 0.764 | 0.939 | 65 | 88% | 65% |
| R8 (outer inferior) | 0.820 | 0.720 | 0.920 | 57 | 79% | 62% |
| R9 (outer temporal) | 0.731 | 0.612 | 0.850 | 65 | 64% | 59% |

Note that the central region for the GCC is missing due to the partial lack of ganglion cells in the foveola.
AUC: area under the curve;
CI: confidence interval;
GCC: ganglion cell complex;
GCL + IPL: ganglion cell layer and inner plexiform layer complex;
RNFL: retinal nerve fiber layer,
cpRNFL: circumpapillary RNFL.

Discussion: This study evaluated the usefulness of macular OCT image segmentation in patients with MS in order to determine the structural changes of the retina of MS patients. Furthermore, the parameter which could discriminate best the eyes of healthy subjects from the eyes of MS patients was determined

Objective markers might be necessary not only for the diagnosis but also the follow-up of neuronal damage in MS, which could help to determine the effect of any possible therapeutical interventions in the future as well. Our results showed that the thickness of the macular ganglion cell complex had the highest sensitivity and specificity to detect axonal loss independent of optic neuritis, outperforming the cpRNFL thickness data provided by the analysis software of the commercially available Stratus OCT device. A strong correlation with disease severity measured by the EDSS score was also obtained in the case of the GCC thickness which implies that this parameter might also be useful in the estimation of disease progression as a surrogate marker. Indeed, Syc et al. have used a similar segmentation methodology to ours extracting four retinal layers (after collapsing the INL+OPL layers) in their recent report and found that the thickness of the GCL+IPL could be a valuable parameter for the longitudinal follow-up of neuronal loss after optic neuritis. Our study has also shown that optic neuritis is followed by a targeted loss of ganglion cells in the macula which can also be objectively assessed by quantitative analysis of OCT macular images.

Several studies have reported the atrophy of the RNFL around the optic disc in patients with MS with and even without optic neuritis in medical history. Our findings confirmed that the mean overall cpRNFL thickness and the cpRNFL thickness in each quadrant is significantly lower in the eyes of MS patients with a history of ON compared to the non-ON-affected eyes and also in comparison with healthy eyes except for the nasal quadrant. However, our results showed that in the non-affected eyes of MS patients the cpRNFL thickness is decreased only in the temporal quadrant compared to healthy eyes. Furthermore, the most pronounced reduction in the thickness of cpRNFL (27% and 17% in the ON-affected and non-affected eyes, respectively) was also observed in the temporal quadrant. These findings are in agreement with previous OCT studies confirming that the fibers of the papillomacular bundle are the most susceptible to damage in ON. One important aspect of this fact, as it has been widely reported in glaucomatous damage, is that the evaluation of the sectoral thickness values could provide the possibility to discriminate the RNFL atrophy caused by glaucomatous damage and other disorders affecting the optic nerve, such as MS.

As a result of neuronal loss, not only the thickness of the cpRNFL is decreased but also the macula was found to be thinner in the eyes of MS patients in previous reports Histopathological studies had qualitatively shown the atrophy of the inner retina in the eyes of MS patients, while atrophy of the outer nuclear layer was not detected. However, no quantitative measurements were performed because of technical difficulties, e.g. the partial post-mortem detachment occurring in the retina in many of the eyes. Lately, some studies evaluating a low number of patients and using OCT technology showed that the thickness of the inner retinal layers is decreased in the eyes of MS patients. However, the reliability of the methodologies used in these studies is not known. Burkholder et al. analyzed a large sample consisting of 530 subjects with RRSM, assessing the volume of the total retina in the inner and outer ETDRS rings (also referred to as pericentral and peripheral macular rings). Their results showed the thinning of the inner and outer retinal ETDRS rings in the eyes of MS patients; however, the local morphological changes of the observed thinning could not be identified as they did not use any segmentation methodology. The OCT image segmentation methodology used in our study allowed the quantification of local retinal changes in patients with MS in vivo. Our results confirmed that the atrophy of the RNFL, GCL+IPL and consequently the GCC is present in the macula of patients with MS even in eyes without ON in previous history. Furthermore, we demonstrated that the outer layers of the retina are not involved in this process. Although it was not an inclusion criterion, all patients had only one episode of ON in the history; therefore, the observed changes were not biased by the number of ON episodes. The thinning of the retina was most pronounced in the inner inferior, inner temporal, outer superior and outer nasal regions.

The average cpRNFL thickness showed the strongest correlation with the thickness of the GCL+IPL and GCC in the macula while a weaker correlation was observed with the thickness of the total retina. Previous studies have also shown that there is a correlation between the mean overall cpRNFL thickness and the thickness of the total retina in the macula, the correlation coefficient was the same as in our results (r=0.68). In the another study, a 10 μm reduction in the mean overall cpRNFL thickness was associated with a 0.2 mm³ reduction of the total macular volume. As the area of the macula—which is imaged using Stratus OCT—is approximately 28.26 mm², the volume of 0.2 mm³ consequently equals to about 7.1 μm in thickness. Our results have shown a similar amount of reduction (7.5 μm) in the total retinal thickness which was accounted for by the reduction of the GCC thickness.

Previously, the mean overall cpRNFL thickness was found to correlate significantly with functional parameters such as EDSS score and contrast sensitivity However, the thickness of the GCL+IPL in the macula was found to correlate better with these functional parameters, which might thus be a better marker of axonal damage. Our results showed good correlation between the EDSS score and the mean overall cpRNFL thickness and also the thickness of the GCL+IPL and the GCC in the macula. However, the ROC analysis revealed that the value most capable of determining the presence of neuronal damage was the weighted mean thickness of the GCC having an AUC value of 0.892 with a cutoff value of 104 μm having the highest sensitivity and specificity. The thickness of the RNFL and GCL+IPL separately showed lower AUCs than the GCC which could be explained with the better reproducibility of the GCC due to the high contrast between the IPL and INL layers. Furthermore, the AUC values observed for the parameters which can be measured using conventional OCT softwares (i.e. the temporal cpRNFL thickness and the average thickness of the total retina expressed as macular volume) were much lower compared to that of the GCC. Despite their lower AUC values we believe these parameters may still be of clinical importance due to their widespread access by all OCT devices. Among the regional thickness values, the thickness of the GCC in the inner temporal region was observed to have the largest AUC value, namely 0.867 (see Table 20).

Although our results showed that the weighted mean GCC thickness may provide a sensitive tool for the assessment of axonal degeneration, care should be taken when interpreting its value as numerous neurodegenerative disorders, such as glaucoma, Alzheimer's disease or Parkinson's disease may also lead to ganglion cell death. The use of regional values could help the differential diagnosis between various forms of neurodegeneration, as glaucoma could presumably lead to an infero-superior pattern of GCC loss in the macula, while according to our results MS is rather leading to a horizontal loss of the GCC most probably due to the loss of the papillo-macular nerve bundle. However, further research is warranted to justify the above hypothesis.

A potential limitation of the study is the relatively low number of MS patients included. Although the size of our study is in accordance with previous similar reports, a larger set of patients may be desired to obtain information with even higher power. Another weakness might be the time-domain OCT technology used which could both limit the accuracy of our measurements due to lower resolution when compared with spectral-domain OCT technology which facilitates wider and finer sampling of the macular regions. However, we have previously shown the high reliability and reproducibility of OCTRIMA measurements obtained with time-domain OCT and even its comparability with higher resolution Fourier-domain OCT measurements.

The results imply that mainly the ganglion cells are affected in MS and changes can be already present in eyes without previous history of ON which could be the result of axonal loss due to the disease process of MS or mild optic neuritis events not accompanied by pain.

By the use of OCT image segmentation, we could also show in vivo that the neuronal damage affects the ganglion cells and not the outer retina, while episodes of ON are resulting in a further pronounced loss of the retinal ganglion cells. Furthermore, our measurements obtained with a custom-built software were shown to be more sensitive compared to standard measurements extracted by the Stratus OCT device (e.g. cpRNFL, total macular volume) and also showed a stronger correlation with physical disability measured by the EDSS. This implies the potential clinical usefulness of the quantification of the macular GCC thickness by OCT image segmentation, which could also facilitate the cost-effective follow-up of neuronal damage due to MS.

In conclusion, we consider that macular OCT image segmentation showing in vivo structural changes of retinal tissue will yield a better insight into macular pathology and therefore should play an important role in the future of the diagnosis and follow-up of neurological diseases affecting the optic nerve, such as multiple sclerosis which influences a continuously increasing number of patients worldwide

Further, given the results from our study, we conclude that obtaining WMT values for the GCC, cpRNFL, GCL+IPL complex, RNFL and TR may be a beneficial tool for diagnosing/evaluating the presence and progression of axonal damage in MS subjects. In our population (or a similar population), a GCCn 104 μm (WMT cutoff point), GCL+IPL complex ro68 μm, RNFL plex rom our study, we conclude that obcan be used to detect the presence of neural loss in MS patients who may benefit from intervention trials to evaluate neuroprotection or repair in MS. We conclude that our results have shown this methodology could have the potential to differentiate MS eyes (w/o previous history of ON) with axonal damage from healthy eyes.

### Optical Coherence Tomography for Retinitis Pigmentosa.

Although the various embodiments have been described above with respect to diabetic retinopathy, the invention is not limited in this regard to only this ocular disorder. Rather, as noted above, the OCT data collection and analysis methods described above can be adapted for purposes of assessing a level of other ocular diseases and disorder, including, but not limited to assessing the structure and function of the macula in patients with retinitis pigmentosa.

Retinitis pigmentosa (RP) is the most common inherited retinal dystrophy, with a worldwide prevalence of approximately 1:4000. The disease is characterized by progressive night blindness and visual field constriction, a rod-cone pattern of electroretinographic (ERG) abnormality (if any ERG remains detectable) and characteristic degenerative retinal changes. Long-term visual prognosis in RP is difficult to predict: some patients maintain acceptable visual function until an advanced age; many do not. The initial degenerative changes occur in the photoreceptors, especially in the midperipheral retina where rod photoreceptor density is maximal. Cone cell death is probably consequent on rod photoreceptor death. As the disease progresses the macula may or may not become involved.

The structure of the macula can be assessed objectively using optical coherence tomography (OCT). Commercially available OCT devices allow limited measurement of the retinal nerve fiber layer (BNFL) thickness around the optic disc along with the thickness of the retina in the macula. Quantitative data about the intraretinal structures can be observed with the use of OCT image processing softwares. Several authors have reported OCT thinning of the photoreceptor layer in patients with RP. However, there is little consistency emerging from studies of inner retinal structure.

Multifocal electroretinography (mfERG) is a noninvasive method that allows assessment of the spatial distribution of the function of central retinal cones. In advanced stages of RP mfERG represents only residual cone activity. The present study correlates the results of mfERG with OCT parameters that include retinal thickness measurements from OCT.

Methods: Patients diagnosed with RP and who had received both OCT and mfERG at the same visit between November 2006 and March 2010 at the Department of Ophthalmology, Semmelweis University, Budapest, Hungary, were retrospectively reviewed. Exclusion criteria were the presence of any other ocular or optic nerve disease, including glaucoma, or of any systemic disease other than controlled hypertension. Exclusion criteria based on OCT imaging were the following: cystoid macular edema, with or without epiretinal membrane formation; a low signal strength (SS) of the OCT images (SS<6); and foveal decentration (center point thickness SD>10%). Twenty-nine eyes of 22 front 57 RP patients were included (16 males and 6 females, median age: 32 years; range: 14 to 63 years). Diagnostic criteria of RP included progressive night blindness and visual field constriction, a rod-cone pattern of ERG abnormality, atrophic optic discs, and intraretinal bone spicule pigmentary deposition (bilateral). Among the study subjects ten had sporadic, one had autosomal recessive, two had autosomal history but no definitive inheritance pattern could be established (either due to lack of information or the small number of relatives). Confirmatory mutational data were not available.

For the OCT control group 17 eyes from 17 age-matched controls were randomly selected from the normative database (median age: 31 years; range: 21 to 59 years). Eligibility criteria for control subjects were best-corrected Snellen visual acuity (VA) of 20/20 and the lack of any ophthalmic, neurologic, or systemic diseases. All control subjects gave informed consent and the study conformed to the tenets of the Declaration of Helsinki. No Institutional Review Board approval was required for the study.

OCT was performed using a time-domain (TD)-OCT device (Stratus OCT; Carl Zeiss Meditec, Dublin, Calif.). Each eye was scanned using the “macular thickness map” protocol, consisting of six radial scan lines centered on the fovea, each having a 6-mm transverse length. The OCT raw data were exported from the device and further processed with optical coherence tomography retinal image analysis (OCTRIMA), which is an interactive, stand-alone application for analyzing TD-OCT retinal images. Segmentation errors were manually corrected using the manual correction tool provided by OCTRIMA.

The thickness values for the RNFL, ganglion cell layer and inner plexiform layer complex (GCL+IPL), inner nuclear layer (INL), outer plexiform layer (OPL), outer nuclear layer (ONL), and the total retina were recorded for each eye in each Early Treatment Diabetic Retinopathy Study (ETDRS) region (see FIG. 14). It is important to note that OCTRIMA measures total retinal thickness between the vitreoretinal border (ILM) and the inner boundary of the second hyperreflective band, which has been attributed to the outer segment/retinal pigment epithelium (OS/RPE) junction, in agreement with histological stud-ies. Moreover, the sublayer labeled as ONL is actually enclosing the external limiting membrane and inner segment (IS), but in the standard 10 μm resolution OCT image this thin membrane cannot be clearly visualized, making the segmentation of the IS difficult. Also, since there is no significant luminance transition between the GCL and the IPL, the outer boundary of the GCL layer is difficult to visualize in the image. Thus, a combined GCL+IPL layer is preferable. To obtain more precise measurements, the INL and OPL were collapsed and measured together for the analyses because the reproducibility of the layers taken separately is worse than that of the collapsed layer. In addition, the intraretinal layers in various eccentricities from the fovea were assessed by calculating the mean thickness of the layers for the central (R1), pericentral (R2-R5), and peripheral (R6-R9) ETDRS regions.

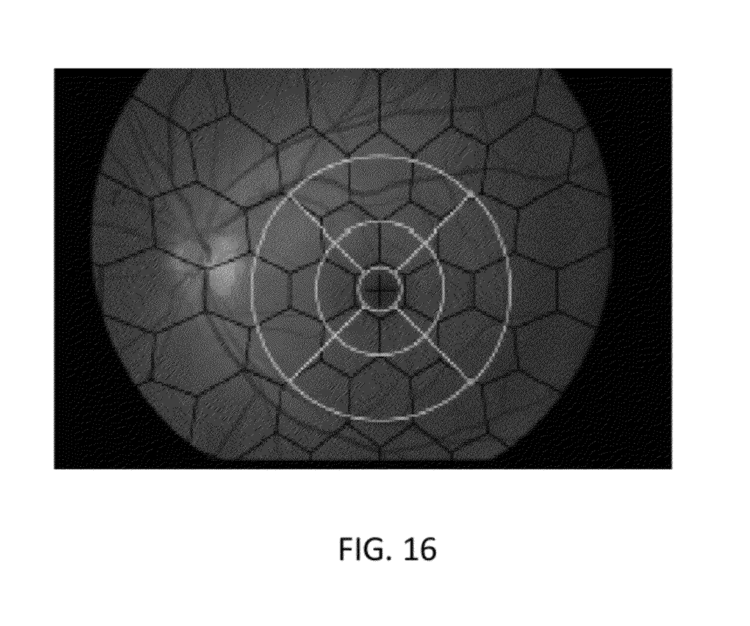

mfERG (RETI-scan; Roland Consult, Stasche & Finger GmbH, Wiesbaden, Germany) was recorded monocularly using ERG-Jet electrodes and a 61-hexagon stimulus according to the guidelines of the International Society for Clinical Electrophysiology of Vision, with a 21-inch video stimulating display (CRT monitor, 75-Hz frame rate, cutoffs: 10-100 Hz) subtending 30° on either side of fixation. A narrow “X” was used for fixation, to cover as little of the central stimulus element as possible. Patients' fixation was continuously monitored using a camera system. Two recordings were obtained, each approximately 4 minutes in duration. Any large eye movements or fixation losses were rejected and the recording was repeated. The retinal area stimulated by the central hexagon was between 0 and 2.5°, by ring 2 between 2.5 and 8°, and by ring 3 between 8 and 15° eccentricity from the fovea on either side. Therefore, the central ETDRS subfield corresponds mainly to the central hexagon area on mfERG and the pericentral ETDRS subfield corresponds mainly to the second ring of hexagons, as shown in FIG. 16.

Both trace array and ring presentation of first-order kernels were performed and evaluated. Since earlier work has shown that the retinal signal occurs within the first 60 ms after stimulation, each trace recording was divided into two epochs: a signal epoch between 15 and 75 ms and a noise epoch between 100 and 150 ms. Because signal to noise ratio (SNR) methods provide a better reliability to discriminate signal from noise in recordings from patients with retinitis pigmentosa, where traces are very attenuated, signal detection based on SNR was used. The root mean square (RMS) amplitude for each of these epochs was calculated as a measure of the magnitude for each epoch. The SNR was calculated by dividing the RMS amplitude of the signal epoch by the mean of the RMS amplitude of the noise epoch at each hexagon, providing further calculation according to eccentricities. The SNR would be close to 1 if a waveform contained no signal with 69% of correct discriminations, and higher SNR values would imply an increased probability that the waveform contains a signal. Accordingly, SNR values were used for the separation of eyes to those with and without detectable mfERG responses, with a threshold of 1.4 as a cutoff for a detectable signal with a discriminability of >95%. Two groups were formed based on the SNR data: one with detectable retinal function (DRF, n=15; median age: 34 years; range: 15 to 63 years) and one with no central retinal function (NCRF, n=14; median age: 32.5 years; range: 14 to 47 years).

Amplitudes and peak times of the P1 components of first-order kernels were measured. For the mfERGs the patient data were compared with the normal age-matched control group of our electrophysiological laboratory (n=50; median age: 31.5 years; range: 23 to 42 years) using mean±2SD values as the criterion for abnormality. Pupils were dilated with cyclopentolate 0.5%; topical anesthesia was one drop of 0.45% oxybuprocaine. Each patient was optimally refracted before testing and then corrected for the viewing distance of 32 cm from the subject's eyes.

Best-corrected VA was recorded in Snellen equivalents and then transformed to logMAR (logarithm of the minimal angle of resolution). The thickness values of the intraretinal layers and the macula and logMAR visual acuities were compared between the three groups using mixed-model ANOVA followed by Newman-Keuls post hoc test. Linear correlation analysis was performed and Pearson correlation coefficients were used to assess the correlation between logMAR visual acuities, disease duration, and the thickness of the measured intraretinal layers. Statistical analyses were performed using commercial statistical analytics software (SPSS15.0; SPSS Inc., Chicago, Ill.; and Statistica 8.0 Software; Statsoft Inc., Tulsa, Okla.). Because of the number (n=14) of comparisons, Bonferroni adjustment was performed for the level of statistical significance, which was set at P<0.0036.

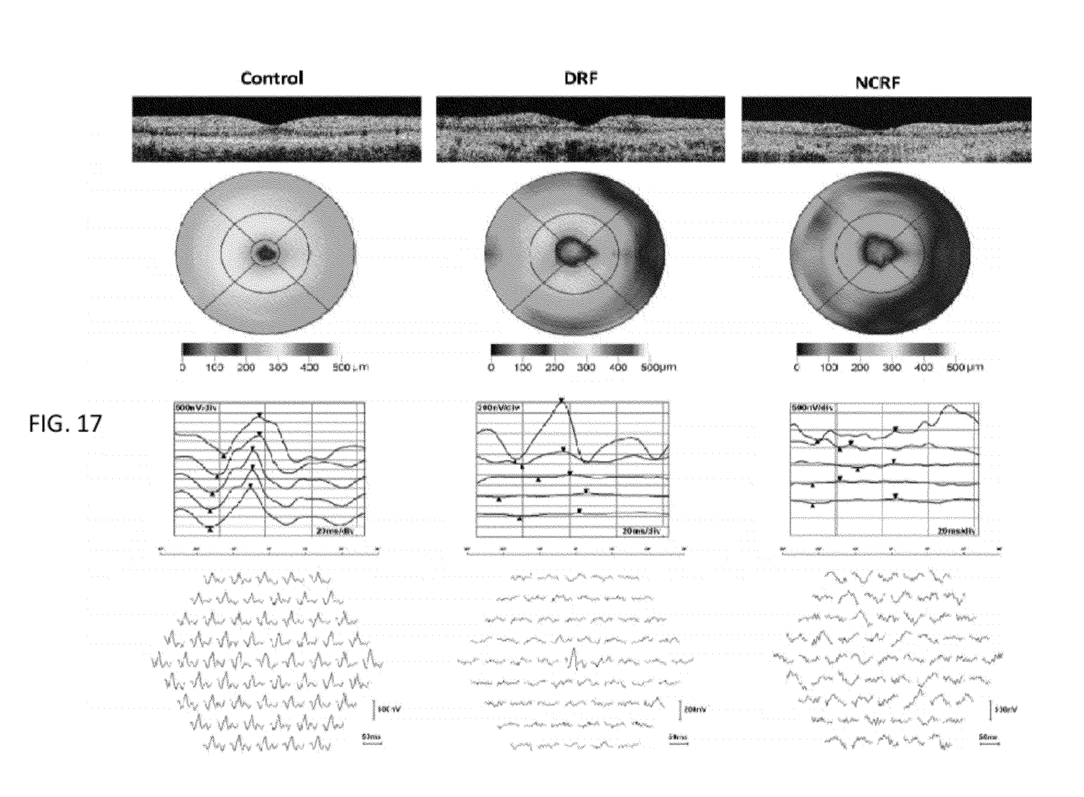

Visual Function: LogMAR VA was significantly worse in the NCRF group compared with that in the DRF group and controls (1.00±0.00, 0.84±0.26, and 0.42±0.22 for the Control, DRF, and NCRF groups, respectively). There was no significant age difference between the groups. A clinical example from all groups is shown in FIG. 17, with corresponding structural and functional results.

FIG. 17 shows a clinical example of a healthy control, an eye from the DRF, and an eye from the NCRF group with respective structural and functional data. The first row shows horizontal cross-sectional OCT scans of the macula. The second row shows corresponding macular thickness map. The third row shows ring presentation (summarized values of traces according to eccentricity). The fourth row shows trace arrays (topographic map of electric activity) of the mfERG recordings (note the differing calibration). Note that the thickness of the macula (of the eye) from the DRF group is decreased in the pericentral and peripheral region compared with the healthy eye. Also, macular thickness (of the eye) from the NCRF group in the central and pericentral area is decreased compared with (the eye from) the DRF group. The ring presentation of the DRF eye indicates an average peak of <10 nV/deg2 in all eccentricities except ring 1 (central hexagon), whereas there is no visible peak in any eccentricities in the NCRF eye. Note also that there is a remarkably diminished central peak surrounded by some (very) low peaks of unusual shape (or delayed peaks) in the trace arrays of the DRF group, but there is no detectable central peak in the NCRF eye (most traces simply reflect background “noise”).

Functional Alterations: Multifocal ERGs were reduced or nondetectable in all patients, with peripheral responses being more affected than central ones. There was no detectable mfERG in the NCRF group in any of the rings, whereas responses to rings 3 to 5 were undetectable in the DRF group. The response densities (RD), P1 amplitudes, P1 peak times, and SNR distributions among the groups are shown in Table 1. Thirteen of 15 eyes (88%) in the DRF group had detectable mfERG components only in response to the central foveal hexagon; two eyes (12%) showed additional detectable mfERG in ring 2. Both the mean RDs and mean P1 values in the DRF group in ring 1 were reduced to 20% of the normal mean. Mean peak time of P1 was within normal timing in the central hexagon.

TABLE 21
Multifocal ERG Characteristics of the Patient Groups and the
Age-matched Normal Control Group

| | Normal | DRF | NCRF |
|---|---|---|---|
| Characteristic | (n = 50) | (n = 15) | (n = 14) |
| RD, nV/deg² | | | |
| Ring 1 | 129.00 (24.02) | 26.60 (9.60) | ND |
| Ring 2 | 84.60 (12.05) | 8.82 (3.32) | ND |
| Ring 3 | 59.30 (9.90) | ND | ND |
| P1 amplitude, μV | | | |
| Ring 1 | 2.00 (0.38) | 0.41 (0.25) | ND |
| Ring 2 | 1.85 (0.27) | 0.20 (0.07) | ND |
| Ring 3 | 1.84 (0.33) | ND | ND |
| P1 peak time, ms | | | |
| Ring 1 | 38.07 (3.30) | 39.60 (4.30) | ND |
| Ring 2 | 35.30 (2.47) | 39.56 (5.70) | ND |
| Ring 3 | 34.60 (1.37) | ND | ND |
| SNR | | | |
| Ring 1 | 3.75 (1.63) | 1.91 (0.38) | 0.94 (0.20) |
| Ring 2 | 6.20 (1.80) | 1.17 (0.26) | 0.97 (0.23) |
| Ring 3 | 5.56 (1.53) | 1.03 (0.12) | 0.71 (0.15) |

Results are expressed as means (±SD).
ND, not detectable.

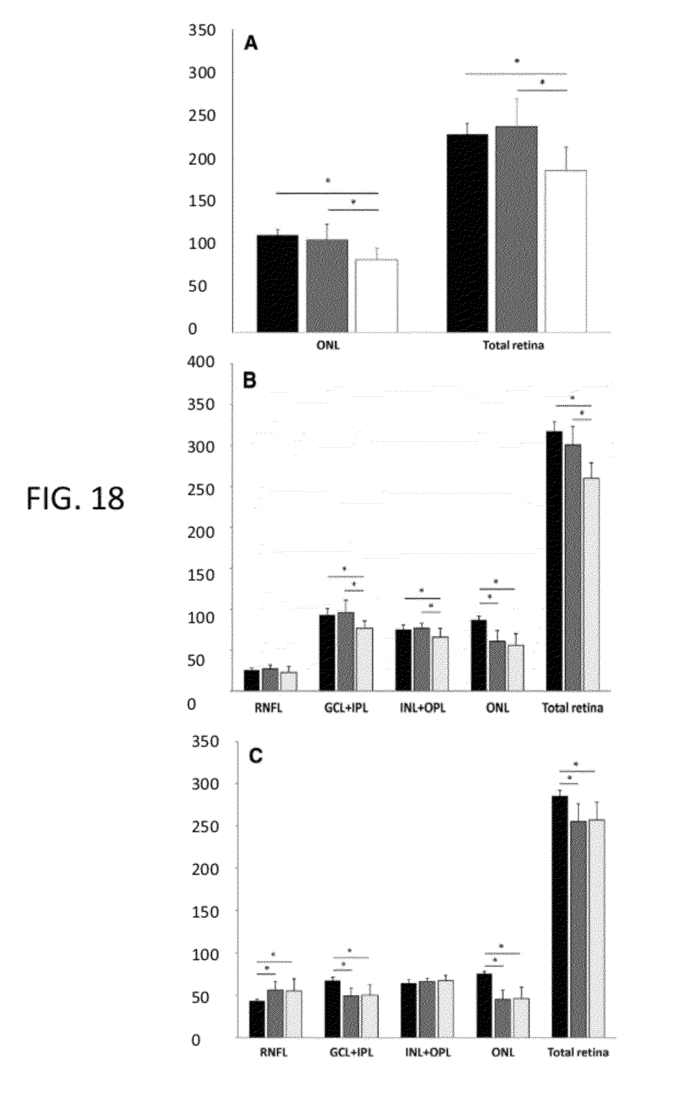

Structural Changes in the Central Region: Total retinal thickness and ONL thickness in the central region did not significantly differ between the DRF group and con-trols. The total thickness of the retina and the ONL was significantly less in the NCRF eyes compared with that in both DRF eyes and controls (see Table 22 and A in FIG. 18)

FIG. 18 shows retinal layer thickness results (A) in the central, (B) in the pericentral, and (C) in the peripheral regions. The black columns are for the Control group, the gray columns are for the DRF (decreased retinal function on mfERG) group, and the white columns are for the NCRF (no central retinal function on mfERG) group. The vertical bars denote the SD of the mean. *Statistically significant difference.

TABLE 22
Regional Thickness of the Intraretinal layers and the Total Retina

| | Thickness (mm), Mean (SD) | | | P Values | | |
|---|---|---|---|---|---|---|
| Variable | Control | DRF | NCRF | DRF vs. Control | NCRF vs. Control | DRF vs. NCRF |

Central Region

| | | | | | | | | |
|---|---|---|---|---|---|---|---|---|
| ONL | 112 (7) | 111 | (16) | 83 | (12) | 0.946 | <0.001* | <0.001* |
| Total retina | 229 (13) | 245 | (26) | 186 | (25) | 0.036 | <0.001* | <0.001* |

Pericentral Region

| | | | | | | | | |
|---|---|---|---|---|---|---|---|---|
| RNFL | 25 (3) | 28 | (4) | 22 | (6) | 0.050 | 0.233 | 0.006 |
| GCL + IPL | 93 (8) | 99 | (14) | 77 | (8) | 0.041 | 0.001* | <0.001* |
| INL + OPL | 75 (6) | 78 | (5) | 66 | (10) | 0.275 | 0.003* | <0.001* |
| ONL | 87 (5) | 60 | (11) | 58 | (16) | <0.001* | <0.001* | 0.371 |
| Total retina | 317 (12) | 305 | (20) | 262 | (19) | 0.205 | <0.001* | <0.001* |

Peripheral Region

| | | | | | | | | |
|---|---|---|---|---|---|---|---|---|
| RNFL | 43 (2) | 57 | (9) | 54 | (14) | <0.001* | 0.002* | 0.396 |
| GCL + IPL | 67 (4) | 50 | (9) | 49 | (11) | <0.001* | <0.001* | 0.522 |
| INL + OPL | 64 (4) | 66 | (4) | 66 | (6) | 0.344 | 0.385 | 0.978 |
| ONL | 75 (3) | 44 | (10) | 46 | (14) | <0.001* | <0.001* | 0.667 |
| Total retina | 285 (7) | 256 | (22) | 255 | (20) | <0.001* | <0.001* | 0.640 |

For statistical analyses, see the text and FIG. 2
*P values are considered statistically significant.

Structural Changes in the Pericentral Region: The ONL was significantly thinner in the pericentral region in both RP groups compared with that in controls, with no difference between patient groups. The GCL+IPL and INL+OPL did not differ significantly between the DRF and control eyes, whereas all these complexes were significantly thinner in the NCRF group compared with those in the DRF group. The RNFL did not differ between groups. The thickness of the retina was significantly less in both patient groups compared with that in control eyes. Furthermore, NCRF eyes had significantly thinner macula in this region compared with that in DRF eyes (see Table 22 and B in FIG. 18).

Structural Changes in the Peripheral Region: The ONL along with the GCL+IPL and the total retina was significantly thinner in the peripheral region in both RP groups compared with those in controls, but did not differ between patient groups. The INL+OPL did not show any significant difference in any of the comparisons. As opposed to the pericentral region, the RNFL in the peripheral region was significantly thicker in both patient groups compared with that in controls, but did not differ significantly between the two patient groups (see Table 22 and C in FIG. 18).

The ONL and the total retina in the central region showed a significant linear correlation with logMAR VA (r=−0.56 and r=−0.59, P=0.003 and P=0.002, respectively) in both patient groups, such that better VA was associated with greater preservation of structure. There was no correlation between lution OCT. The findings are consistent with the histologic demonstration of shortening of ROS in RP.

Although OCT demonstration of thinning of the photoreceptors is a consistent observation, studies describing changes in the INL and OPL are less conclusive, In the present study, there was no detectable change in the thickness of the middle layers of the peripheral macula (i.e., the INL and OPL), which were considered as a middle retinal complex (i.e., INL+OPL) for the segmentation analysis to obtain more precise measurements. Secondary degeneration affecting inner retinal cells as a consequence of photoreceptor degeneration could result in simultaneous reactive hyperplasia of the Muller glial cells that might counterbalance the putative loss of INL neurons. However, the INL+OPL complex was significantly thinner in the pericentral region in the NCRF group, perhaps due to a different degree of the involuntary reaction of the INL and hypertrophic glial response. Previous histopathologic and OCT studies have shown no significant changes in the INL and OPL layers. In one study, the sectioned maculae of 21 postmortem eyes with RP were analyzed and it was found that a major part of the cells of the INL remained intact even in eyes with severe RP. In another study, the thickness of the INL was found to be close to normal on spectral-domain OCT images measured with a manual segmentation procedure aided by a computer program.

The ganglion cell and inner plexiform layer complex in the pericentral region of those patients with detectable mfERG was preserved in the present study and thinning was observed only in the peripheral region. In contrast, the GCL+IPL complex was thinner in both peripheral and pericentral regions of those with undetectable mfERG responses. This may reflect more prominent photoreceptor damage and transneuronal degeneration in patients with undetectable mfERG responses giving a more pronounced thinning of the inner retinal layers, among them the ganglion cells. Since cone density is higher in the more central regions, photoreceptor loss and consecutive transneuronal degeneration may be less evident in the central and pericentral macula in less advanced stages of the disease than it is in the peripheral macula. Accordingly, the ganglion cells of the pericentral region are still seemingly preserved in eyes in which the underlying photoreceptors have already been reduced due to RP. However, the results of the NCRF group suggest that the ganglion cells in the pericentral region also degenerate with disease progression. The results of post-mortem morphometric studies, showing a significant reduction in the number of ganglion cells in eyes with moderate and severe RP, are consistent with the present in vivo results showing the thinning of ganglion cell layer in advanced RP. In contrast, some studies have observed the thickening of the inner retina in association with the loss of ONL, whereas other studies found the thickness of the GCL+IPL close to normal using images further processed with custom algorithms.

A pronounced thickening was observed for the RNFL in the peripheral region of both groups, which could be due to an early phase of epiretinal membrane formation. Although it was not among the aims of the present study to quantify this process, peripheral FRMS not involving the fovea (according to the exclusion criteria) were present in 41% and 42% in the DRF and NCRF groups, respectively. Based on histopathology, these membranes are formed by proliferation of fibrous astrocytes on the surface of the optic nerve head that progress to more peripheral areas. For this reason, it is not surprising that the retina was thicker in the region closer to the optic disc, whereas in the pericentral region there was only a slight thinning detectable in the NCRF group, most probably due to the loss of the ganglion cells and their corresponding axons. Previous studies using OCT for the measurements of the peripapillary RNFL thickness showed conflicting results, reporting both thinning and thickening of the retinal nerve fiber layer in the eyes of patients with RP.

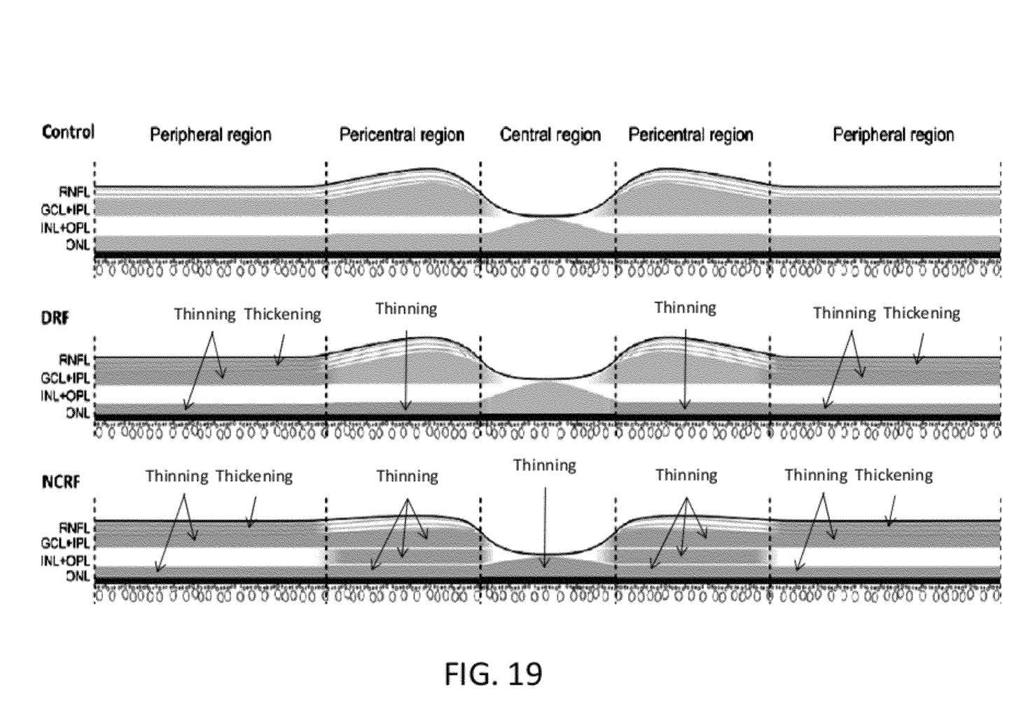

The presumed trend cited earlier of gradual degeneration involving the ganglion cells due to prior photoreceptor loss is also supported by the total retinal thickness results, Specifically, in those eyes with detectable mfERG response, the thickness of the ONL in the central region representing the total retina is preserved. Moreover, the thickness of the retina in the pericentral region is significantly thinner than that in controls but at the same time thicker than that in those eyes with undetectable mfERG response, whereas the two RP groups have the same reduced thickness in the peripheral macula. A schematic drawing of the presumed trend of neurodegeneration occurring in RP based on the above-cited results can be seen in FIG. 19.

FIG. 19 shows a schematic drawing of the observed pathologic changes in the macula of patients with RP. The top row shows normal structure of a healthy macula. The middle row shows schematic drawing of a macula in the DRF (decreased retinal function on mfERG) group. The bottom row shows an image of a macula in the NCRF (no central retinal function on mfERG) group. Areas of thinning and thickening are denoted. Note that RNFL thickening occurs early in the peripheral region, whereas the pericentral ganglion cell ayer (GCL+IPL) is affected only in the NCRF group despite the thinning of the pericentral ONL already in the DRIP group.

Visual acuity in the present study was significantly worse in the NCRF group compared with that in both the DRF group and controls. The logMAR value showed a good correlation with the thickness of the ONL and the macula in the central region. Previous studies have reported no correlation between central foveal thickness and logMAR VA; however, the correlation between macular photoreceptor outer segment-RPE thickness and logMAR VA was strong.

One group has previously shown a correlation between the spatial extent of mfERG preservation and the radius of the paracentral ring of hyperautofluorescence in RP patients with good visual acuity. There was also spatial concordance between sensitivity loss measured by high spatial resolution fine matrix mapping and the size of the high-density hyperautofluorescent ring, Inner/outer photoreceptor segment junction disruption across the hyperautofluorescent ring has been previously observed, whereas outside the ring the inner/outer photoreceptor segment junction and the ONL appeared to be absent. Those data, and the correlation between structure and function found in the present study, point toward the need for a multimodal approach, to better understand the pathologic processes in RP.

There are some potential shortcomings of our study. On mfERG ring analysis, the “ring 1” central response is often the noisiest because it relates to a single stimulus hexagon and reliable amplitude measurement may be difficult, which could have biased the distinction between the two groups; we believe the use of SNR provides a relatively objective way to deal with this problem. Spatial correspondence between mfERG stimulus and OCT scans has been one of the major issues in all studies comparing structure and function. In our study, we used a 61 hexagonal-element pattern array that covered a 30° radius of the central visual field. The diameter of the first three mfERGs (only 19 hexagons) span 15° radius. Therefore, the OCT scan length (˜20°) presumably covers approximately the diameter of these first three concentric mfERG rings. Consequently, the foveal and parafoveal retina are properly matched. A partial mismatch is present in the parafoveal region since the OCT scan slightly extends to ring 4. Thus, we note that the matching between the mfERG stimulus and the OCT scans needs to be improved in future studies by developing a proper geometrical model in a simulation study.

Regarding OCT technology, first, the ONL contains the inner segments of the photoreceptors that are interconnected with the outer segments of the photoreceptors and pigment epithelial cells. As previously mentioned, ultrahigh resolution and spectral-domain technologies facilitate a more precise delineation of the RPE and inner segment-outer segment junction of the macular photoreceptors, although the valuable retrospective database reaching back to 2006 used for the study made it impossible to include Fourier domain-OCT analyses. Although OCTRIMA is able to extract the RPE layer in time-domain OCT, there is much variability in the segmentation of the RPE outer boundary due to the lower resolution of deeper structures extracted by the OCT device. Therefore, those measurements were not included. Although the INL and OPL layers could be better resolved by spectral-domain OCT technology, TD-OCT and SD-OCT have been shown to give comparable results when OCTRIMA is used. Also, the results may be influenced by a combination of the quality of the scans (i.e., distinctiveness of the layers) and the segmentation algorithm performance. For example, a systematic deviation in the RNFL/GCL border could yield patients with thinner GCL+IPL, but thicker RNFL layers, although we believe the scan quality control in OCTRIMA along with previously demonstrated high reproducibility of OCTRIMA makes this error relatively unlikely. Future studies will benefit from higher resolution imaging; an increase in the size of the patient population studied will also be of importance along with longitudinal data.

Currently, there is no effective treatment for RP. The use of retinal implants is a possible avenue for the restoration of vision to the blind. However, the efficacy of such implants depends on the cells of the inner nuclear layer and the ganglion cell layer being functionally intact. The quantitative structural data provided by OCT image segmentation could be a valuable tool in effective selection of patients, where the integrity of the ganglion cell layer is a prerequisite for potential use of retinal prostheses and could act as a useful outcome measure in therapeutic interventions

Given the results from our study, we conclude that obtaining a thickness map for the GCL+IPL complex in both peripheral and pericentral regions may be a beneficial tool for evaluating the integrity of the inner retina in subjects with RP that could facilitate the selection of the candidates for a subretinal implant. Therefore, we will compare thickness maps of the ganglion cell layer and inner plexiform complex (GCL+IPL) and retinal nerve fiber layer (RNFL) of a RP subject to a reference map. If the value of the RP subject's thickness map exceeds the value of the reference map at the corresponding map coordinate by a preset multiple of standard deviation (SD), an abnormal thickness (i.e. lack of integrity) is indicated and it is included in the “abnormal classification (e.g. not a candidate for transplantation)”. The threshold level for the abnormal/non-intact region should be lower or higher than that for the reference. 2SDs will be used to define cutoffs for the upper and lower levels of normative values.

### Other Embodiments

It is to be understood that while the invention has been described in conjunction with the detailed description thereof, the foregoing description is intended to illustrate and not limit the scope of the invention. Other aspects, advantages, and modifications are within the scope of the following claims.

All references cited herein, are incorporated herein by reference. Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the Disclosure is provided to allow the reader to quickly ascertain the nature of the technical disclosure. It is submitted with the understanding that it will not be used to interpret or limit the scope or meaning of the following claims.

What is claimed is:

1. A method of characterizing biological tissues, comprising:
providing measurement data of biological tissue for a patient obtained using an optical coherence tomography system;
processing the measurement data to obtain layer data, the layer data comprising an identification and characterization of structural and optical properties of different cellular layers in the retinal tissue, the at least one of structural and optical properties comprising thickness values, reflectance values, scattering coefficients, and texture measures for the different retinal layers; and
comparing the layer data to pre-determined criteria for the at least one of structural and optical properties associated with neural loss in patients with a neurodegenerative disease;
characterizing a degree of neural loss in the biological tissue for the patient based on the comparing.

2. The method or claim 1, wherein the neural loss comprises axonal damage.

3. The method of claim 1, wherein the pre-determined criteria comprises stored correlation data identifying a relationship between at least a portion of the layer data and the degree of neural loss.

4. The method of claim 1, wherein the pre-determined criteria comprises stored threshold data identifying threshold values for at least a portion of the structural and optical properties corresponding to at least one amount of neural loss.

5. The method of claim 4, wherein the stored threshold data comprises pre-defined thicknesses for at least one of a ganglion cell layer and inner plexiform layer complex (CGL+IPL), a ganglion cell complex (GCC), retinal nerve fiber layer (RNFL), circumpapillary retinal nerve fiber layer (cpRNFL), and total reflectance (TR).

6. The method of claim 5, wherein the stored thickness data comprises at least the pre-defined thicknesses for the CGL+IPL and the GCC.

7. The method of claim 1, wherein the neurodegenerative disease is selected from the group consisting of muscular dystrophy, Alzheimer's disease, and Parkinson's disease.

8. A method of diagnosing and monitoring axonal damage due to muscular dystrophy patient, comprising:
providing measurement data of biological tissue for a patient obtained using an optical coherence tomography system;
processing the measurement data to obtain layer data, the layer data comprising at least thicknesses of a ganglion cell layer and inner plexiform layer complex (CGL+IPL) and a ganglion cell complex (GCC); and
characterizing the degree of axonal damage due to muscular distrophy in the patient based on the layer data.

9. The method of claim 8, wherein the characterizing further comprises:
obtaining stored correlation data identifying a relationship between at least GCL+IPL and GCC values and a degree of axonal damage in population of muscular dystrophy patients; and
determining the degree of axonal damage in the patient due to muscular distrophy based on a comparison of the layer data and the stored correlation data.

10. The method of claim 8, wherein the characterizing further comprises:
obtaining stored threshold data identifying at least one set of GCL+IPL and GCC values corresponding to a specific degree of axonal damage due to muscular dystrophy; and
determining whether the patient corresponds to the specific degree of axonal damage based on a comparison of the layer data and the stored threshold data.

11. The method of claim 8, wherein the characterizing further comprises:
obtaining previously stored layer data for the patient data; and
determining a current status of the patient based on a comparison of the layer data and the stored layer data.

12. A method of characterizing biological tissue, comprising:
providing measurement data of biological tissue for a patient obtained using an optical coherence tomography system;
processing the measurement data to obtain layer data, the layer data comprising an identification and characterization of structural and optical properties of different cellular layers in the retinal tissue, the at least one of structural and optical properties comprising thickness values, reflectance values, scattering coefficients, and texture measures for the different retinal layers; and
classifying the biological tissue based on layer data and reference data to indicate a suitability of the biological tissue for a subretinal implant based on the classification.

13. The method of claim 12, wherein the classifying comprises:
generating a patient thickness map of the biological tissue based on at least a ganglion cell layer and inner plexiform complex (GCL+IPL);
comparing the patient thickness map to a reference thickness map; and
labeling the biological tissue as unsuitable for the subretinal implant if a difference between the patient thickness map and the reference thickness map exceeds a pre-defined threshold.

14. The method of claim 13, wherein the predefined threshold comprises at least one standard deviation.

15. The method of claim 13, wherein the generating further comprises generating the patient thickness map based on at least the GCL+IPL and retinal nerve fiber layer (RNFL).
